(19) United States
(12) Patent Application Publication
(10) Pub. No.: US 2015/0119267 A1
(43) Pub. Date: Apr. 30, 2015

(54) INHIBITION OF COLONY STIMULATING FACTOR-1 RECEPTOR SIGNALING FOR THE TREATMENT OF BRAIN CANCER

(71) Applicant: SLOAN-KETTERING INSTITUTE FOR CANCER RESEARCH, New York, NY (US)

(72) Inventor: Johanna Joyce, New York, NY (US)

(21) Appl. No.: 14/394,765

(22) PCT Filed: Apr. 15, 2013
(86) PCT No.: PCT/US13/36628

§ 371 Date: Oct. 16, 2014

Related U.S. Application Data
(60) Provisional application No. 61/643,022, filed on May 4, 2012.
(60) Provisional application No. 61/624,861, filed on Apr. 16, 2012.

(30) Foreign Application Priority Data
May 4, 2012 (US) PCT/US2012/036589
May 4, 2012 (US) PCT/US2012/036630

(51) Int. Cl. C12Q 1/68; G01N 33/50; G01N 33/574

(52) U.S. Cl. CPC C12Q 1/6886; G01N 33/57407; G01N 33/5011; C12Q 2600/106; C12Q 2600/158; G01N 2500/10; G01N 2800/52
USPC 506/9; 435/6.11; 435/6.13; 435/6.12

(57) ABSTRACT

The present invention provides a method of screening brain tumor patients for treatment with inhibitor of CSF-1R, based on differential gene expression including adrenomeduUin (ADM), arginase 1 (ARG1), clotting factor F13A1, mannose receptor C type 1 (MRC1/CD206), and protease inhibitor SERPINB2 after treatment with the inhibitor. Based on the same differential gene expression profile, the present invention also provides a method of screening a compound to treat brain cancer.

### CROSS-REFERENCE TO RELATED APPLICATIONS

This application claims the priority of U.S. Application No. 61/482,723, filed May 5, 2012; U.S. Application No. 61/643,022, filed May 4, 2012; International Application No. PCT/US 12/36630, filed May 4, 2012; International Application No. PCT/US 12/36589, filed May 4, 2012 and U.S. Application No. 61/624,861, filed Apr. 16, 2012. The entire contents and disclosures of the preceding applications are incorporated by reference into this application.

### FIELD OF THE INVENTION

This invention relates to the use of inhibiting colony stimulating factor (CSF)-1 receptor signaling in the treatment of human diseases. In one embodiment, this invention relates to the use of inhibitor of colony stimulating factor (CSF)-1 receptor for the treatment of brain cancer.

### BACKGROUND OF THE INVENTION

Among the considerable challenges in treating gliomas is substantial genetic and tumor cell heterogeneity that results in aberrant activation of multiple signaling pathways. Non-cancerous stromal cells represent genetically stable therapeutic targets that can play critical roles in tumor development and progression. Macrophages are one such cell type that is associated with poor patient prognosis and treatment response in many cancers, including gliomas.

Several experimental approaches have been used to either ablate macrophages or target their tumor-promoting functions in various mouse models of cancer. One strategy is to inhibit colony stimulating factor (CSF)-1 receptor (CSF-1R) signaling, which has been shown to deplete macrophages and reduce tumor volume in different xenograft models, including intratibial bone tumors and non-small cell lung cancer. A paracrine CSF-1/EGF signaling loop has additionally been shown to be important in promoting breast cancer and glioblastoma multiforme (GBM) invasion.

Glioma-associated macrophages could originate from microglia, the resident macrophage population in the brain, and/or be recruited from the periphery. The relative contributions of resident microglia versus recruited macrophages to gliomagenesis have not been extensively addressed. Both of these macrophages will be referred collectively herein as tumor-associated macrophages (TAMs). It is currently not known whether therapeutic targeting of TAMs in glioblastoma multiforme (GBM) represents a viable strategy.

Glioblastoma multiforme (GBM), the most common and aggressive primary brain tumor, is renowned for its terminal prognosis, emphasizing the urgency of developing new effective therapies. Hence, there is a need for investigating therapeutic targeting of TAMs and the use of CSF-1R inhibitor for the treatment of brain cancer.

### SUMMARY OF THE INVENTION

Macrophages are dependent upon colony stimulating factor (CSF)-1 for differentiation and survival; therefore, an inhibitor of its receptor, CSF-1R, was used to target macrophages in a mouse glioma model, the RCAS-PDGF-B-HA/Nestin-Tv-a;Ink4a/Arf^{-/-} mouse model of gliomagenesis.

CSF-1R inhibition dramatically increased survival in mice and regressed established GBMs. Tumor cell apoptosis was significantly increased, and proliferation and tumor grade markedly decreased. Surprisingly, TAMs were not depleted in the CSF-1R inhibitor-treated tumors. However analysis of gene expression in TAMs isolated from treated tumors revealed a decrease in alternatively activated/M2 macrophage polarization markers, consistent with impaired tumor-promoting functions. These gene signatures were also associated with improved survival specifically in the proneural subtype of patient gliomas. Collectively, these results establish macrophages as valid therapeutic targets in gliomas, and highlight the clinical potential for CSF-1R inhibitors in GBM.

### BRIEF DESCRIPTION OF THE FIGURES

FIG. 1 shows CSF-1R inhibition specifically targets macrophages in the PDG model, significantly improves survival and decreases glioma malignancy.

FIG. 1A shows tumors from PDG mice (n=3) were sorted into a mixed population of live cells (DAM, purified tumor cells (GFP⁺) and macrophages (CD11b⁺G1l⁻). Cd11b and Tv-a were used as cell type-specific control genes for macrophages and tumor cells respectively. Expression was depicted relative to the live cell fraction, normalized to Ubc for each sample. FIG. 1B shows BLZ945 blocks macrophage survival in culture as determined by MTT assay, with a comparable effect to CSF-1 deprivation. FIG. 1C shows BLZ945 was tested against independent PDG tumor cell lines and the PDGFR-dependent human U-87 MG glioma cell line using MTT assay. Concentrations of BLZ945 up to 6700 nM had no effect. The results depict triplicate wells from one of 3 representative experiments. FIG. 1D shows experimental design for long-term survival trial: PDG mice were injected with RCAS-PDGF-B-HA between 5-6 weeks of age to induce tumor formation, and were randomly assigned to vehicle (20% captisol, n=22) or BLZ945 (200 mg/kg, n=14) treatment groups at 2.5 weeks post-injection. Mice were dosed once daily until they developed symptoms or reached the trial endpoint. FIG. 1E shows symptom-free survival curves. FIG. 1F shows the vehicle and BLZ945 groups were graded histologically (n=14, 13 respectively). P values were obtained using unpaired two-tailed Student's t-test in FIGS. 1B-C, Log Rank (Mantel-Cox) test in FIG. 1E, and Fisher's exact test in FIG. 1F. Data are presented as mean±SEM. *P<0.05, ***P<0.001.

FIG. 2 shows CSF-1R inhibition blocks tumor growth and effectively regresses established gliomas.

FIG. 2A shows experimental design: PDG mice underwent MRI scans to assess tumor volume and were randomly assigned to vehicle or BLZ945 groups, with follow-up MRI as depicted. FIG. 2B shows mean tumor volume over the time course for mice whose starting tumor volume was 4.5-40 mm³ (n=11 per group). FIG. 2C shows mean tumor volume over the time course for mice whose starting tumor volume was >40 mm³ (BLZ945 Large, n=18). FIG. 2D shows representative images of T2-weighted MRI scans from beginning and endpoint of the trial. Dashed line indicates region of interest used to calculate tumor volume. FIG. 2E shows waterfall plots depicting change in tumor volume at endpoint relative to starting tumor volume for each individual mouse. Horizontal dashed lines indicate 30% decrease in tumor volume. P values were obtained using unpaired two-tailed Student's t-test. Data are presented as mean±SEM. **P<0.01, ***P<0.001.

FIG. 3 shows short-term BLZ945 treatment results in reduced tumor grade and proliferation, and increased apoptosis.

FIG. 3A shows representative H&E images from the 7-day trial depicting grade IV/GBM (vehicle) and tumor response (BLZ945). FIG. 3B shows representative images from 7-day trial stained for Olig2 (tumor cells) BrdU, cleaved caspase-3 (CC3), and DAPI. White arrows indicate rare BrdU⁺Olig2⁺ cells in BLZ945 groups. FIG. 3C shows quantitation of total DAPI⁺ cells per tumor. FIG. 3D shows percentage of Olig2⁺ cells relative to total DAPI⁺ cells. FIG. 3E shows percentage of proliferating BrdU⁺Olig2⁺ cells. FIG. 3F shows percentage of apoptotic CC3⁺ cells relative to total DAPI⁺ cells (n=5-6 per group). Circles represent individual mice. Scale bar, 50 μm. P values were obtained using unpaired two-tailed Student's t-test; ns=not significant, *P<0.05, **P<0.01, ***P<0.001.

FIG. 4 shows CSF-1R inhibition signature reveals changes in macrophage polarization and predicts survival advantage in proneural GBM patients.

FIG. 4A depicts Volcano plot showing differentially expressed genes comparing BLZ945 to vehicle (7 days treatment, n=8 each). 205 downregulated and 52 upregulated genes were differentially expressed in the BLZ945 group. FIG. 4B shows a lasso logistic regression model was trained on expression data in FIG. 4A, identifying 5 genes differentiating BLZ945 and vehicle. BLZ945 downregulates expression of Mrc1/CD206 in BMDMs in vitro, determined by flow cytometry (FIG. 4C) and qRT-PCR (FIG. 4D). FIG. 4E shows primary glioma cultures were cultured +/−BLZ945, and CD45⁺CD11b⁺ cells were analyzed for Mrc1 expression by flow cytometry, n=6. FIG. 4F shows glioma cells were co-cultured with BMDMs that were either unstimulated or pre-conditioned with GCM. Co-cultures were treated +/−BLZ945 and tumor cell cycle entry evaluated, revealing an increase when cultured with GCM-preconditioned BMDMs, which was blocked by BLZ945. FIG. 4G shows glioma cell-conditioned media (GCM) induces primary bone marrow-derived macrophages (BMDM) proliferation and protects BMDMs from BLZ945-induced cell death, assessed by MTT assay (representative experiment shown, n=3). For comparison, BMDMs were cultured in non-conditioned media supplemented with CSF-1. FIG. 4H shows The Cancer Genome Atlas (TCGA) proneural patients were classified into “BLZ945-like” and “Vehicle-like” classes using the lasso signature shown in FIG. 4B. “BLZ945-like” classified patients show increased median survival of 10 months. FIG. 41 shows hazard ratios (HR) and confidence intervals (CI) for the lasso regression signature determined for each subtype of TCGA and Combined (Murat, Phillips, Freije, and Rembrandt) datasets. HR means are plotted with associated 95% CI: HRs with a CI that does not cross 1.0 are considered significant. The proneural subtype alone showed significant association with survival in both TCGA and Combined datasets. P values were obtained using unpaired two-tailed Student's t-test in FIGS. 4C-G, Chi-squared test in FIG. 4H, and Wald's test in FIG. 41. Data are presented as mean±SEM. *P<0.05, **P<0.01, ***P<0.001.

FIG. 5 shows macrophage numbers are increased in a mouse model of gliomagenesis compared to normal brain.

FIG. 5A shows cerebrum/forebrain from uninjected Nestin-Tv-a;Ink4a/Arf^{-/-} mice (normal brain) or grade IV tumors (GBM) from symptomatic RCAS-PDGF-B-HA/Nestin-Tv-a;Ink4a/Arf^{-/-} (PDG) mice were processed to a single cell suspension with papain for flow cytometry (n=5 each). There was a significant increase in CD45⁺ leukocytes from 3.6±0.6% to 13.1±2.0%. CD11b⁺ myeloid cells/macrophages accounted for the overwhelming majority of leukocytes (89.9-98.5% of CD45⁺ cells), with a 3.8-fold increase in CD45⁺CD11b⁺ cells in the tumors (12.7±2.0%) compared to normal brain (3.3±0.5%), and no differences in the populations of CD45⁺CD11b⁻ cells. FIG. 5B shows normal brain or GBM tissue sections from symptomatic PDG mice were immunofluorescently co-stained for CSF-1R, CD68 (macrophages), and DAPI. FIG. 5C shows normal brain and GBM tumors (n=3 each) were used for RNA isolation, cDNA synthesis, and qPCR. Assays were run in triplicate and expression normalized to ubiquitin C (Ubc) for each sample. Expression is depicted relative to normal brain. FIG. 5D shows normal brain or GBM tissue sections from symptomatic PDG mice were stained for CSF-1R in combination with the macrophage markers F4/80 and CD11b. F4/80, CD11b, and CD68 were also examined in combination with Iba-1 (macrophages/microglia). DAPI was used for the nuclear counterstain. Scale bar, 50 μm. Data are presented as mean±SEM. P values were obtained using unpaired two-tailed Student's t-test; *P<0.05; **P<0.01.

FIG. 6 shows BLZ945 significantly decreases the viability of macrophages in culture but has no effect on glioma cell line proliferation or neurosphere formation.

FIG. 6A shows chemical structure of the CSF-1R inhibitor BLZ945. FIG. 6B shows Western blot analysis of primary BMDMs, which were cultured in the absence of CSF-1 for 12 hours prior to stimulation, followed by CSF-1 addition for the time points indicated. This results in a progressive increase in CSF-1R phosphorylation that is effectively inhibited by 67 nM BLZ945. In lane 1, marked by *, BMDMs were continuously cultured with CSF-1. The same dose of BLZ945 (67 nM) blocks wild-type C57BL/6 BMDMs as shown in FIG. 1B. FIG. 6C shows Nestin-Tv-a,Ink4a/Arf^{-/-} BMDM survival, as determined by the MTT assay, which is comparable to the effects of CSF-1 deprivation from the culture. FIG. 6D shows BLZ945 blocks survival of the CRL-2467 microglia cell line. FIG. 6E shows the potency of BLZ945 was tested against multiple PDG tumor cell lines derived from independent mice in culture using the MTT assay. There was no effect of BLZ945, even at concentrations up to 6700 nM, which is 100× the IC50 for CSF-1R inhibition in cell-based assays. The results depict mean±SEM of triplicate wells from one of 3 representative experiments in FIGS. 6C-E. FIG. 6F shows BLZ945 does not affect the number or size of secondary neurospheres (NS) derived from 3 independent mice bearing PDG tumors, which were seeded in duplicate wells for each condition. Data are presented as mean±SEM. P values were obtained by comparing each concentration of BLZ945 to the untreated control at the end of the experiment using unpaired two-tailed Student's t-test; **P<0.01, ***P <0.001 in FIGS. 6C-D; for all the comparisons in FIG. 6E and FIG. 6F, there were no significant differences.

FIG. 7 shows BLZ945 crosses the blood-brain barrier, is well tolerated for long-term treatments, and significantly reduces tumor grade.

FIG. 7A tests whether BLZ945 could cross the blood-brain barrier. Tumor-bearing PDG mice were treated with a single dose of 200 mg/kg BLZ945 by oral gavage and sacrificed at different time points post-dosing as indicated to determine BLZ945 pharmacokinetics (PK). Plasma, the right tumor-bearing hemisphere (tumor), and the left contralateral hemisphere of the brain (contralateral brain) were snap frozen for subsequent analysis of BLZ945 concentration in the tissue (n=3 mice per time point). At 2 hours post-BLZ945 administration, the concentration of the drug in the brain was similar to levels in the plasma and decreased thereafter. Notably, the BLZ945 concentration in the contralateral non-tumor bearing hemisphere of the brain was comparable to the level achieved in the tumor at all time points tested, indicating that the drug is able to effectively cross the blood-brain barrier and was not solely due to the potentially selective disruption of this barrier within the tumor. Data are presented as mean±SEM. FIG. 7B shows BLZ945 is well-tolerated for up to 26 weeks. Mean weight for female and male mice over the 26-week time course of the long-term survival trial depicted in FIG. 1E. Mice were divided by treatment group: vehicle and BLZ945. The BLZ945 treatment group was also subdivided into mice that became symptomatic and were taken off trial (symptomatic, n=3 females, n=2 males) or those mice that survived to the trial endpoint of 26 weeks (endpoint, n=3 females, n=6 males). At this time point these mice did not show any obvious macroscopic symptoms. FIG. 7C shows tumor grade in both cohorts of mice from the long-term survival trial. All vehicle treated mice at end-stage had high-grade tumors. In contrast, BLZ945 treated animals had significantly less malignant tumors. This group was then stratified into mice sacrificed as symptomatic during the trial (n=4), from those still asymptomatic when sacrificed at the 26-week endpoint (n=9). In each BLZ945 group, there was still a significant decrease in tumor grade compared to the vehicle cohort. Remarkably there were no detectable lesions in 55.6% of the asymptomatic mice at end-stage. P values were obtained using Fisher's exact test. *P<0.05, ***P<0.001.

FIG. 8 shows tumor growth is inhibited in individual mice in response to BLZ945. PDG mice underwent MRI scans to assess tumor volume between 4-5 weeks post-injection and were randomly assigned to vehicle (20% captisol) or BLZ945 (200 mg/kg) treatment groups. FIG. 8A-C shows tumor volume over the time course for individual mice (from FIG. 2). Mice whose starting tumor volume was 4.5-40 mm³ were treated with vehicle (FIG. 8A) or BLZ945 (FIG. 8B) (n=11 per group). FIG. 8C shows a third group of mice with tumor volume >40 mm³ was treated with BLZ945 (BLZ945 Large, n=18). Initial tumor volume in this group ranged from 48.7-132.3 mm³. A vehicle cohort with tumor volume >40 mm³ was not included for comparison because those mice would not have survived to the trial endpoint.

FIG. 9 shows BLZ945 treatment inhibits intratumoral CSF-1R phosphorylation. Tumors were harvested from mice after 3 days of treatment with either BLZ945 or vehicle. Samples were biochemically fractionated as described, and CSF-1R phosphorylation was assessed by western blotting (FIG. 9A). FIG. 9B shows a significant reduction in CSF-1R phosphorylation, but no significant change in total receptor levels, as determined by quantitation of the phosphorylated and total CSF-1 receptor bands using ImageJ software. n=5 mice per group. Data are presented as mean±SEM. P values were obtained using unpaired two-tailed Student's t-test; **P<0.01, ns, not significant.

FIG. 10 shows decreased angiogenesis and evidence of pronounced tumor response in BLZ945-treated tumors. FIG. 10A shows tissues from the 7 day trial were graded histologically (n=5-10 per group). While all of the vehicle treated mice had high-grade tumors, with 89% having grade IV GBMs, all of the BLZ945 treated mice exhibited a tumor response already evident at d3. This response was characterized by a clear depopulation of tumor cells, with maintenance of the stroma and leukocytic infiltrate (representative image shown in FIG. 3A). FIG. 10B shows representative images of tumors from 7 day BLZ945 trial stained for CD31 (endothelial cells, red), smooth muscle actin (SMA, pericytes, green), and DAPI (blue). FIG. 10C shows quantitation of the microvessel density (CD31 count relative to the total tumor area). FIG. 10D shows the average vessel length (CD31 length relative to the CD31 count), and FIG. 10E shows pericyte coverage (percentage of SMA staining overlapping CD31⁺ staining). There were no significant differences in pericyte coverage among the treatment groups. Circles represent individual mice (n=5-6 per group). Scale bar, 50 μm. P values were obtained using unpaired two-tailed Student's t-test; **P<0.01, ***P<0.001.

FIG. 11 shows increased phagocytosis in BLZ945-treated tumors. Representative images of tumors from the short-term BLZ945 trial stained for CD11b (macrophages), cleaved caspase-3 (CC3), Olig2 and DAPI were shown in FIG. 11A. White arrows indicate apoptotic tumor cells (CC3⁺Olig2⁺) that have been engulfed/ phagocytosed by CD11b⁺ macrophages. Gray arrowheads indicate apoptotic tumor cells (CC3⁺Olig2⁺) that are in close contact with but have not been phagocytosed by CD11b⁺ macrophages; these types of interactions were not counted for phagocytic index or capacity. FIG. 11B shows phagocytic index calculated as the mean percentage of CC3⁺Olig2⁺ cells that had been engulfed by CD11b⁺ macrophages per mouse. FIG. 11C shows phagocytic capacity calculated as the mean percentage of CD11b⁺ macrophages that had engulfed CC3⁺Olig2⁺ cells per mouse. Circles represent individual mice (n=5-6 per group). Scale bar, 50 μm. P values were obtained using unpaired two-tailed Student's t-test; *P<0.05, **P<0.01, ***P<0.001.

FIG. 12 shows CSF-1R inhibition depletes normal microglia but does not affect the number of TAMs in treated gliomas. Normal non-tumor bearing Nestin-Tv-a;Thk4a/Arf^{-/-} mice were treated with vehicle (20% captisol; n=3) or 200 mg/kg BLZ945 (n=2) once per day for 7 days, and the following day the animals were sacrificed and the brains prepared for flow cytometry with collagenase III digestion. FIG. 12A shows representative flow cytometry plots and FIG. 12B shows the quantitation of CD11b⁺Ly6G⁻ microglia and CD11b⁺Ly6G⁺ myeloid cells. Data are presented as mean±SEM. FIG. 12C shows representative images of tumors (upper panel) and adjacent hippocampus (lower panel) from the short-term BLZ945 trial stained for CD68 (macrophages, red) and DAPI (blue). FIG. 12D shows quantitation of the mean number of CD11b⁺ macrophages per 63× field of view within the tumor per mouse and FIG. 12E shows the percentage of Iba1⁺ macrophages that are CSF-1R⁺ within the tumor. Circles represent individual mice (n=5-6 per group). P values were obtained using unpaired two-tailed Student's t-test; *P<0.05.

FIG. 13 shows gene expression profiling of BLZ945-treated TAMs, demonstrating a downregulation of alternatively activated/M2 polarization markers with no change in classically activated/M1 polarization markers.

FIG. 13A shows representative flow cytometry plots and gating strategy for sorting CD11b⁺Gr-1⁻ TAMs from tumors treated with vehicle or BLZ945 for 7 days. FIG. 13B shows supervised clustering of 257 differentially expressed genes between BLZ945 and vehicle treated mice (n=8 per group). BLZ945 treatment resulted in a downregulation of 205 genes and an upregulation of 52 genes. These genes were used to train the Support Vector Machine (SVM) in FIG. 16. FIG. 13C show Gene set enrichment analysis (GSEA), revealing that targets of Egr2, a transcription factor downstream of CSF-1R signaling, were downregulated in BLZ945 treated TAMs. Of the differentially expressed genes, ten including the five identified using lasso regression in FIG. 4B, were found to be associated with alternative/M2 macrophage activation (see Table 2) (FIG. 13D). FIG. 13E shows that classically activated/M1 macrophage markers represented in the 257 gene list were not differentially expressed following BLZ945 treatment, with the exception of IL-1 beta, which was significantly upregulated (P=4.5×10⁻⁴).

FIG. 14 shows analysis of immune cell infiltration in BLZ945-treated tumors. Tumors from the short-term BLZ945 trial were processed on day 7 to a single cell suspension with collagenase III for flow cytometry. Quantitation of immune cell infiltration by flow cytometry is shown for each vehicle or BLZ945 treatment group. (vehicle n=5, BLZ945 treated n=6). Data are presented as mean±SEM. P values were obtained using unpaired two-tailed Student's t-test and all comparisons between treatment groups were not significant.

FIG. 15 shows primary glioma cultures are composed of heterogeneous cell types. Primary glioma cell cultures were prepared from GBM as described in methods. At P3, cells were stained for Nestin to reveal the presence of tumor cells in combination with the macrophage marker CD11b as well as the astrocyte marker GFAP. DAPI was used for the nuclear counterstain. Scale bar, 50 μm.

FIG. 16 shows “BLZ945-like” lasso and support vector machine classes demonstrate a survival advantage in a Proneural specific, G-CIMP independent manner.

FIG. 16A shows lasso logistic regression signature identified in FIG. 4B was used to classify TCGA GBM patients into “BLZ945-like” and “Vehicle-like” classes. The regression model was trained on the mouse TAM expression data, restricting to genes with known human homologues that were present in TCGA total tumor expression data. Expression data for MRC1 was not present in the TCGA data, thus the lasso regression utilized ADM, ARG1, F13A1, and SERPINB2 expression values to classify patients into mock “Vehicle-like” or mock “BLZ945-like” classification groups. “BLZ945-like” classified TCGA patients demonstrated an increased median survival in the Proneural subtype (10 months). This increase in survival was not evident in other subtypes of GBM in the TCGA data. Although, ADM and F13A1 contribute the strongest weights to the signature, exclusion of any member of the 4-gene signature abrogated the described survival benefit (data not shown).

FIG. 16B shows GBM patients from the Murat, Freije, Phillips, and Rembrandt databases were subtyped as described and binned into one dataset. As in FIG. 16A, the lasso logistic regression signature was used to classify patients in the Combined datasets. “BLZ945-like” classified patients demonstrated a survival advantage in only the proneural subtype (6.5 months). While there was a significant decrease in median survival using the “BLZ945-like” class in the Neural subtype from the TCGA dataset in FIG. 16A, this effect was not replicated in the Combined datasets for Neural patients.

FIG. 16C shows a support vector machine (SVM) was trained as described and used to classify TCGA patients into “BLZ945-like” and “Vehicle-like” classification. “BLZ945-like” classified TCGA patients showed a Proneural specific survival advantage (7.6 months).

FIG. 16D shows an SVM was trained and used as in FIG. 16C to classify patients in the Combined datasets into “BLZ945-like” and “Vehicle-like” classification. “BLZ945-like” classified patients showed a Proneural specific survival advantage (31.5 months).

FIG. 16E sought to determine if the survival advantage offered by the “BLZ945-like” signature was due to an enrichment of Glioma CpG Island Methylator Phenotype (G-CIMP) patients, which have previously been shown to be associated with improved overall survival. Proneural patients with available methylation data (see methods) were classified into “BLZ945-like” and “Vehicle-like” classes using the lasso signature as in FIG. 16A. G-CIMP-positive patients were removed from the analysis. “BLZ945-like” classified patients still demonstrated a significant median survival increase among Proneural non G-CIMP patients (10.4 months). All P values were obtained using a Chi-squared test, and all significant P values are indicated in bold.

### DETAILED DESCRIPTION OF THE INVENTION

The following terms shall be used to describe the present invention. In the absence of a specific definition set forth herein, the terms used to describe the present invention shall be given their common meaning as understood by those of ordinary skill in the art.

As used herein, the expression “tumor-associated macrophages (TAMs)” refers collectively to microglia and macrophages.

As used herein, “BMDM” refers to bone marrow-derived macrophages.

As used herein, “CSF” refers to colony stimulating factor; “CSF-1R” refers to colony stimulating factor-1 receptor.

As used herein, “GBM” refers to glioblastoma multiforme.

As used herein, “GCM” refers to glioma cell-conditioned media.

As used herein, “PDG” refers to PDGF-driven gliomas, using the RCAS-PDGF-B/Nestin-Tv-a;Ink4a/Arf^{-/-} mouse model of gliomagenesis.

As used herein, “TCGA” refers to The Cancer Genome Atlas.

As used herein, “therapeutic reagent” or “regimen” is meant any type of treatment employed in the treatment of cancers, including, without limitation, chemotherapeutic pharmaceuticals, biological response modifiers, radiation, diet, vitamin therapy, hormone therapies, gene therapy, surgery etc.

c-FMS is the cellular receptor for CSF-1 (M-CSF). The extracellular domains of the receptor are characterized by the presence of five immunoglobulin-like domains and a single transmembrane segment. Inside the cell, the transmembrane domain is joined to the tyrosine kinase domain by a juxtamembrane domain, which bears a number of regulatory phosphorylation sites. The structure of the c-FMS tyrosine kinase domain has been determined in Apo form and co-liganded with small molecule inhibitors of different chemotypes. c-FMS is an attractive target for drug discovery because it appears to play a pivotal role in the regulation of macrophage function. Both the extracellular (and in particular the purported CSF-1-binding site) and the intracellular tyrosine kinase domains have been targeted in the generation of therapeutics.

There are a number of potentially therapeutic scenarios for which a potent and specific c-FMS inhibitor might be successfully deployed. The presence of large numbers of macrophages at sites of inflammation, such as the rheumatoid synovium, immune-mediated nephritis, inflammatory bowel disease, coronary disease, sarcoidosis and chronic obstructive pulmonary disease, inter alia, places mediators of macrophage function, such as CSF-1, at the very heart of therapeutic intervention in a wide range of inflammatory diseases.

The role of macrophages in the facilitation of tumourigenesis and their collusion with tumor cells to suppress immune response has become apparent only recently and the nexus between the inflammatory response and the initiation, growth and metastatic spread of tumor cells remains the focus of many current studies in tumor immunology. It has been shown that direct inhibition of c-FMS by inhibiting the expression of CSF-1 by antisense oligonucleotides or antibodies, or of its receptor by siRNA or inhibition of kinase activity all lead to significant changes in the growth of grafted tumors and their cellularity.

The present invention has shown that the CSF-1R inhibition is a potent strategy to block malignant progression, regress established GBMs and dramatically enhance survival in a preclinical model of gliomagenesis. There are several potential clinical implications of these findings. First, increased macrophage infiltration correlates with malignancy in human gliomas, as shown here in the PDG model, supporting therapeutic targeting of TAMs in patients. Second, depletion is not strictly necessary for effective macrophage-targeted therapy as it is shown that alteration of TAM tumor-promoting functions can significantly affect malignancy. Third, it is possible that proneural gliomas in particular are dependent on TAMs, as indicated by the preclinical data presented herein and suggested by the prognostic advantage associated with the gene signatures found specifically in patients of this subtype. As such, it is reasonable to predict that models of other GBM subtypes may also respond similarly to CSF-1R inhibition. Finally, myeloid cells, including macrophages, have been implicated in blunting chemotherapeutic response in breast cancer models and in promoting re-vascularization and tumor growth following irradiation in GBM xenograft models. Thus, it would be logical to consider CSF-1R inhibitors in combination with therapies directed against the cancer cells in gliomas.

The experiments disclosed below employ one CSF-1R inhibitor as an example. Thus, the present invention is not limited to the use of the particular CSF-1R inhibitor presented herein. One of ordinary skill in the art would readily recognize that other CSF-1R inhibitors, or other methods of inhibiting CSF-1R signaling would also be applicable in the methods of the present invention.

To date, small molecules targeting CSF-1R have been designed to bind (at least in part) to the ATP-binding site and no allosteric binders to the receptor have been disclosed. Two broad classes of inhibitors are apparent: those that bind to the kinase in the so-called type I conformation which are thus ATP-competitive and those that bind to the kinase in the type II conformation which are largely non-competitive with ATP. Many different structural motifs have been reported as CSF-1R inhibitors (26). Representative examples of CSF-1R inhibitors include, but are not limited to, CYC10268, a pyrazine series (Cytopia); AZ683, 3-amido-4-anilinocinnolines, Cinnoline, pyridyl and thiazolyl bisamide series, anilide series (all developed by AstraZeneca); ABT-869 (Abbott Laboratories); ARRY-382 (Array BioPharma); JNJ-28312141, heteroaryl amides, quinolinone series, pyrido-pyrimide series (all developed by Johnson and Johnson); GW2580 (Glaxo Smith Kline); quinoline derivatives including Ki20227 (Kirin Brewery); 7-azaindole series, PLX3397 (Plexxikon); 1,4-disubstituted pyrrolo-[3,2-c]pyridine derivative (Korea Institute of Science and Technology); and a benzothiazole series (Novartis).

In addition to these small molecule inhibitors, additional means to inhibit CSF-1 signaling include anti-CSF-1 or anti-CSF-1R antibodies. One of ordinary skill in the art would readily generate and use an anti-CSF-1 or anti-CSF-1R antibody for a desired purpose. Antibodies may include, but are not limited to, isolated antibodies, monoclonal antibodies, and fragments of antibodies. Representative examples of anti-CSF-1 antibodies include, but are not limited to, IMC-CSF (ImClone), 7H5.2G10 (Deposit No. DSM ACC2922; Hoffmann-La Roche), and MCS100 (Novartis). See Sherr et al., 1989; Ashmun et al., 1989; Kitaura et al., 2008; WO 2011/107553; and WO 2009/112245.

Another method of CSF-1 signaling inhibition is by antisense oligonucleotide or small interfering RNA (siRNA) directed against CSF-1 or CSF-1R. Using standard techniques or readily available materials in the art, one of ordinary skill in the art would readily generate and use antisense oligonucleotide or siRNA directed against CSF-1 or CSF-1R. See, for examples, Aharinejad et al., 2004 and 2009; and Abraham et al, 2010.

In one aspect of the present invention, there is provided a method of identifying or monitoring the effects of a therapeutic agent or regimen on a brain cancer patient. According to this method, a selected therapeutic agent or treatment regimen is administered to the patient. In one embodiment, the therapeutic agent or regimen comprises or results in signaling inhibition of CSF-1 and/or CSF-1R. In another embodiment, the therapeutic agent or regimen comprises the use of CSF-1 signaling inhibition and another cancer treatment generally known in the art. Periodically during and/or after administration of the agent or during and/or after completion of the therapeutic regimen, a sample containing myeloid cells of the subject is examined for expression of genes that show differential expression as shown herein.

In one embodiment, there is provided a method of determining whether a brain cancer patient would be responsive to treatment with a therapeutic agent or regimen comprising inhibition of CSF-1 signaling. The method comprises the steps of treating said patient with the therapeutic agent or regimen; isolating myeloid cells from said patient; and determining expression of one or more genes in said myeloid cells, said genes include adrenomedullin (ADM), arginase 1 (ARG1), clotting factor F13A1, mannose receptor C type 1 (MRC1/CD206), and protease inhibitor SERPINB2, wherein differential gene expression in said myeloid cells from treated patient as compared to myeloid cells from a patient treated with control reagent or regimen would indicate that said patient would be responsive to treatment with the therapeutic agent or regimen. Inhibition of CSF-1 signaling can be accomplished by one of the methods discussed above for targeting CSF-1 or CSF-1R. In one embodiment, CSF-1 signaling inhibition is accomplished by the use of a CSF-1R inhibitor. In another embodiment, the therapeutic regimen comprises method of CSF-1 signaling inhibition and another generally known method of cancer treatment, such as chemotherapeutic pharmaceuticals, biological response modifiers, radiation, diet, vitamin therapy, hormone therapies, gene therapy, surgery etc.

In another embodiment, the present invention also provides uses of the differential gene expression disclosed herein to determine whether a brain cancer patient would be responsive to treatment with a therapeutic agent or regimen comprising inhibition of CSF-1 signaling.

In general, gene expression can be determined by any method generally known in the art, such as PCR or microarray. In one embodiment, gene expression in said myeloid cells further includes expression of one or more genes such as CD163, Cadherin 1 (CDH1), Heme oxygenase 1 (HMOX1), Interleukin 1 receptor type II (IL1R2), and Stabilin 1 (STAB1). In another embodiment, gene expression in said myeloid cells further includes expression of one or more genes as listed in Table 2. In one embodiment, gene expression for ADM, ARG1, F13A1, and MRC1/CD206 are downregulated in the myeloid cells from the treated patient. In another embodiment, gene expression for SERPINB2 is upregulated in the myeloid cells from the treated patient.

Data presented herein also indicate that differential expression of genes listed above is also related to survival of the cancer patients; therefore, the above method would also be useful in monitoring or predicting the prognosis of the treated patients. For example, patients found to already have evidence of the aforementioned better prognosis gene signature(s) in either a tumor biopsy, or myeloid cells/macrophages directly isolated from said tumor could be expected to further improve in prognosis following treatment (for example, with a CSF-1R inhibitor). Alternatively, patients that do not have evidence of said gene signature(s) prior to treatment might be expected to respond more avidly to the treatment, as monitored by changes in said gene signature(s). In either scenario, the gene signature(s) described herein are expected to have an important role in patient stratification and management prior to, and during treatment (for example, CSF-1R inhibitor therapy). For example, patients can be biopsied prior to CSF-1R inhibitor treatment, and then monitored for treatment efficacy as determined by changes in the aforementioned gene signature(s). Those patients whose gene signature changes would be predicted to have an improved prognosis, and in this regard, this assay could have powerful predictive and prognostic value.

In one embodiment, the complete gene signature provided the most robust separation between patient groups. In another embodiment, either ADM or F13A1 as single gene is also capable of stratifying patient groups by survival. Patients with lower expression of either ADM or F13A1 had better survival outcome compared to patients with high levels of either gene. Thus, in one embodiment, the gene signature can be reduced to analysis of either ADM or F13A1, and important predictive value can still be attained.

In one embodiment, the myeloid cells are macrophages, for example, tumor-associated macrophages, bone marrow-derived macrophages, or peripheral macrophage precursors/monocytes.

In one embodiment, the brain cancer is primary brain cancer such as astrocytoma, oligodendroglioma, neuroblastoma, medulloblastoma or ependydoma. In another embodiment, the brain cancer is a mixed glioma, for example, a malignant tumor that contains astrocytes and oligodendrocytes. In another embodiment, the brain cancer is glioma, including high-grade glioblastoma multiforme. In yet another embodiment, the glioma molecular subtype is proneural. In another embodiment, the brain cancer could include metastatic brain cancer.

In another aspect, the present invention provides a screening method for identifying a cancer therapeutic agent or regimen useful for the treatment of brain cancer. This method can be employed to screen or select from among many pharmaceutical reagents or therapies for the treatment of individual or groups of brain cancers. According to this method, a selected therapeutic agent or treatment regimen is administered to a mammalian test subject having a cancer. The test subject is desirably a research animal, e.g., a laboratory mouse or other. Periodically during and after administration of said agent or regimen, a sample containing cells of the test subject is examined and a gene expression profile is generated.

In one embodiment, the present invention provides a method of screening for a therapeutic reagent or regimen that is useful for treating brain cancer, wherein the therapeutic reagent or regimen comprises inhibition of CSF-1 signaling. The method comprises the steps of treating a subject with the therapeutic reagent or regimen; and determining expression of one or more genes in myeloid cells obtained from such subject, said genes include adrenomedullin (ADM), arginase 1 (ARG1), clotting factor F13A1, mannose receptor C type 1 (MRC1/CD206), and protease inhibitor SERPINB2, wherein differential gene expression in myeloid cells from subject treated with the therapeutic reagent or regimen as compared to myeloid cells from subject that is treated with a control reagent or regimen would indicate that said therapeutic reagent or regimen is useful for treating brain cancer. Inhibition of CSF-1 signaling can be accomplished by any method discussed above for targeting CSF-1 or CSF-1R. In one embodiment, CSF-1 signaling inhibition is accomplished by the use of a CSF-1R inhibitor. In another embodiment, the therapeutic regimen comprises method of CSF-1 signaling inhibition and another generally known method of cancer treatment, such as chemotherapeutic pharmaceuticals, biological response modifiers, radiation, diet, vitamin therapy, hormone therapies, gene therapy, surgery etc.

In another embodiment, the present invention also provides uses of the differential gene expression disclosed herein to screen for a therapeutic reagent or regimen for treating brain cancer, wherein the therapeutic reagent or regimen comprises inhibition of CSF-1 signaling.

In general, gene expression can be determined by any method generally known in the art, such as PCR or microarray. In one embodiment, gene expression in said myeloid cells further includes expression of one or more genes such as CD163, Cadherin 1 (CDH1), Heme oxygenase 1 (HMOX1), Interleukin 1 receptor type II (IL1R2), and Stabilin 1 (STAB1). In another embodiment, gene expression in said myeloid cells further includes expression of one or more genes as listed in Table 2. In one embodiment, gene expression for ADM, ARG1, F13A1, and MRC1/CD206 are downregulated in the myeloid cells from the treated patient. In another embodiment, gene expression for SERPINB2 is upregulated in the myeloid cells from the treated patient.

In one embodiment, the myeloid cells are macrophages, for example, tumor-associated macrophages, bone marrow-derived macrophages, or peripheral macrophage precursors/monocytes.

In one embodiment, the brain cancer is glioma, including high-grade glioblastoma multiforme. In another embodiment, the glioma molecular subtype is proneural. In yet another embodiment, the brain cancer could include metastatic brain cancer, or primary brain cancers such as astrocytoma, oligodendroglioma, neuroblastoma, medulloblastoma or ependydoma. In another embodiment, the brain cancer is a mixed glioma, for example, a malignant tumor that contains astrocytes and oligodendrocytes.

The present invention also provides kits that can be used to detect the expression of genes that show differential expression as shown herein. Accordingly, kits are provided that can be used in the monitoring or screening assays disclosed herein. For example, the kit may include a microarray or nucleic acid primers and probes for the detection of one or more genes that show differential expression as shown herein. The kits can include instructional materials disclosing means of use of the compositions in the kit. The instructional materials can be written, in an electronic form (such as a computer diskette or compact disk) or can be visual (such as video files). One skilled in the art will appreciate that the kits can further include other agents to facilitate the particular application for which the kit is designed.

The invention will be better understood by reference to the experimental details which follow, but those skilled in the art will readily appreciate that the specific experiments detailed are only illustrative, and are not meant to limit the invention as described herein, which is defined by the claims which follow thereafter.

Throughout this application, various references or publications are cited. Disclosures of these references or publications in their entireties are hereby incorporated by reference into this application in order to more fully describe the state of the art to which this invention pertains. It is to be noted that the transitional term “comprising”, which is synonymous with “including”, “containing” or “characterized by”, is inclusive or open-ended and does not exclude additional, un-recited elements or method steps.

### EXAMPLE 1

### Materials and Methods

### Mice

All animal studies were approved by the Institutional Animal Care and Use Committee of Memorial Sloan-Kettering Cancer Center. The Nestin-Tv-a;Ink4a/Arf^{-/-} mouse model (mixed strain background) has been previously described (1, 2). Wild-type (WT) C57BL/6 mice and β-actin-GFP (C57BL/6) mice (3) were purchased from Charles River Laboratories and Jackson Laboratories respectively.

### Intracranial Injections

The initiation of tumors with RCAS-PDGF-B-HA in adult mice has been previously described (4, 5). Briefly, mice were fully anesthetized with 10 mg/ml ketamine/1 mg/ml xylazine and were subcutaneously injected with 50 μl of the local anesthetic 0.25% bupivacaine at the surgical site. Mice were intracranially injected with 1 μl containing 2×10⁵ DF-1:RCAS-PDGF-B-HA cells between 5-6 weeks of age using a fixed stereotactic apparatus (Stoelting). Injections were made to the right frontal cortex, approximately 1.5 mm lateral and 1 mm caudal from bregma, and at a depth of 2 mm.

To investigate cell type specific expression of CSF-1 and CSF-1R in flow cytometric sorted cell populations, tumors were initiated in mice with RCAS-PDGF-B-HA-SV40-eGFP (RCAS-PDGF-GFP) as previously described (6). Nestin-Tv-a;Ink4a/Arf^{-/-} pups were injected with 1 μl of DF-1:RCAS-PDGF-B-GFP cells on post-natal day 2 into the left cortex between the eye and ear.

### CSF-1R Inhibitor And Treatment

The CSF-1R inhibitor was obtained from the Novartis Institutes for Biomedical Research (Emeryville, Calif.). The drug was formulated in 20% captisol at a concentration of 12.5 mg/ml. The vehicle control, 20% captisol, was processed in the same manner. For CSF-1R inhibitor studies, mice were dosed with 200 mg/kg BLZ945 or vehicle (20% captisol) by oral gavage once per day. To determine if the drug was able to cross the blood-brain barrier, tumor-bearing mice were treated with a single dose of the CSF-1R inhibitor and sacrificed at different time points post-treatment. Plasma, and the left (contralateral) and right (tumor-bearing) hemispheres of the brain were snap frozen in liquid nitrogen for subsequent analysis of CSF-1R inhibitor concentrations in the tissue. For long-term survival studies, dosing begun at 17 days/2.5 weeks post-injection of RCAS-PDGF-B-HA. For the fixed time-point studies, mice underwent MRI scans at 4-5 weeks post-injection of RCAS-PDGF-B-HA, as previously described (5). To determine tumor volume, regions of interest (ROI) were circumscribed on T2 weighted images and their corresponding area in mm² was multiplied by the slice height of 0.7 mm. The total tumor volume is the sum of the ROI volume in each slice, and the volume for the first and last slice in which the tumor appears is halved to approximate the volume of a trapezoid. When tumor volume was in the range of 4.5-40 mm³, animals were randomly assigned to treatment groups. A third cohort of mice with tumors larger than 40 mm³ was also treated with the CSF-1R inhibitor (denoted as BLZ945 Large). A size-matched vehicle treated cohort was not included for this larger starting tumor burden because these mice would not have been able to survive to the trial endpoint.

### Mouse Sacrifice And Tissue Harvest

Mice were euthanized at defined time points as described in the figure legends or when they became symptomatic from their tumors, which included signs of poor grooming, lethargy, weight loss, hunching, macrocephaly, or seizures.

To isolate tissues for snap freezing in liquid nitrogen, mice were euthanized by carbon dioxide asphyxiation or fully anesthetized with avertin (2,2,2-tribromoethanol, Sigma) and cervically dislocated prior to tissue harvest. For flow cytometry, mice were fully anesthetized with avertin and transcardially perfused with 20 ml of PBS. The brain was then isolated and the tumor macro-dissected from the surrounding normal tissue. For proliferation analysis, mice were injected intraperitoneally with 100 mg/g of bromodeoxyuridine (BrdU; Sigma) 2 hours prior to sacrifice. To isolate tissues for frozen histology, mice were fully anesthetized with avertin, transcardially perfused with 10 ml of PBS, followed by 10 ml of 4% paraformaldehyde in PBS (PFA). The brain was post-fixed in PFA overnight at 4° C. while other tissues were cryopreserved in 30% sucrose at 4° C. After post-fixation, the brain was then transferred to 30% sucrose and incubated at 4° C. until the brain was fully equilibrated and sank to the bottom of the tube (typically 2 to 3 days). All tissues were then embedded in OCT (Tissue-Tek) and 10 μm cryostat tissue sections were used for all subsequent analysis.

### Histology, Immunohistochemistry, And Analysis

For grading of tumor malignancy, hematoxylin and eosin (H&E) staining was performed, and the tissues were blindly scored by an independent neuropathologist.

For immunofluorescence, 10 μm thick frozen sections were thawed and dried at room temperature and then washed in PBS. For standard staining protocol, tissue sections were blocked in 0.5% PNB in PBS for at least 1 hour at room temperature or up to overnight at 4° C., followed by incubation in primary antibody in 0.25% PNB for 2 hours at room temperature or overnight at 4° C. Primary antibody information and dilutions are listed in Table 6. Sections were then washed in PBS and incubated with the appropriate fluorophore-conjugated secondary antibody (Molecular Probes) at a dilution 1:500 in 0.25% PNB for 1 hour at room temperature. After washing in PBS, tissue sections were counterstained with DAPI (5 mg/ml stock diluted 1:5000 in PBS) for 5 minutes prior to mounting with PROLONG GOLD ANTIFADE mounting media (Invitrogen).

For angiogenesis and proliferation analysis, tissue sections were first subjected to citrate buffer-based antigen retrieval by submerging in antigen unmasking solution (0.94% v/v in distilled water; Vector Laboratories) and microwaving for 10 minutes on half power, followed by cooling to room temperature for at least 30 minutes. For angiogenesis analysis, tissues were then washed in PBS and blocked with mouse Ig blocking reagent (Vector Laboratories) according to the manufacturer's instructions for 1 hour at room temperature. For proliferation analysis, after antigen retrieval, tissue sections were incubated with 2M HCl for 15 minutes at room temperature to denature DNA and then in neutralizing 0.1M sodium borate buffer (pH 8.5) for 5 minutes. After PBS washes, the rest of the staining was performed according to standard protocol.

For staining for phagocytosis analysis, 10 μm thick frozen sections were thawed and dried at room temperature and then washed in PBS. Tissue sections were blocked in 0.5% PNB in PBS for at least 1 hour at room temperature, followed by incubation in rabbit anti-cleaved caspase-3 primary antibody diluted 1:500 in 0.5% PNB overnight at 4° C. The next day, slides were washed 6 times for 5 minutes in PBS prior to incubation with goat-anti-rabbit Alexa568 secondary antibody (1:500 in 0.5% PNB) for 1 hour at room temperature. Tissue sections were then washed 6 times for 5 minutes in PBS and blocked overnight at 4° C. in a new buffer of 5% donkey serum, 3% bovine serum albumin, and 0.5% PNB in PBS. The following day, slides were incubated for 2 hours at room temperature with the next set of primary antibodies: rabbit anti-Olig2 (1:200) and rat anti-CD11b (1:200) diluted in 5% donkey serum, 3% bovine serum albumin, and 0.5% PNB in PBS. Slides were washed 6 times for 5 minutes in PBS prior to incubation with donkey-anti-rabbit Alexa647 (1:500) and donkey-anti-rat Alexa488 (1:500) secondary antibodies in 0.5% PNB for 1 hour at room temperature. Tissue sections were then washed 4 times for 5 minutes in PBS prior to staining with DAPI (5 mg/mL stock diluted 1:5000 in PBS) for 5 minutes, washed twice more in PBS for 5 minutes, and mounted with PROLONG GOLD ANTIFADE mounting media (Invitrogen). Co-staining for CSF-1R (first primary antibody) and Iba1 (second primary antibody) was also performed in series in the same manner, with the addition of citrate buffer based antigen retrieval at the outset.

Tissue sections were visualized under a Carl Zeiss Axioimager Z1 microscope equipped with an Apotome. The analysis of immunofluorescence staining, cell number, proliferation, apoptosis, and colocalization studies were performed using TISSUEQUEST analysis software (TissueGnostics) as previously described (7). Overviews of tissue sections from gliomas stained for angiogenesis analysis were generated by TissueGnostics acquisition software by stitching together individual 200× images. All parameters of angiogenesis were quantitated using METAMORPH (Molecular Devices), as previously described (8). For analysis of phagocytosis, 15 randomly selected fields of view from within the tumor were acquired using the 63× oil immersion objective (total magnification 630×) and the Apotome to ensure cells were in the same optical section. Positive cells were counted manually using VOLOCITY (PerkinElmer) and were discriminated by the presence of a DAPI⁺ nucleus. Apoptotic cells were counted as those that had cytoplasmic cleaved caspase-3 (CC3)⁺ staining and condensed nuclei. A cell was considered to have been engulfed by a macrophage when it was surrounded by a contiguous CD11b⁺ ring that encircled at least two-thirds of the cell border. The numbers of mice analyzed are specified in the figure legends.

### Protein Isolation And Western Blotting

Mice were treated with the CSF-1R inhibitor or vehicle and sacrificed 1 hour following the final dose and tumors were harvested. Samples were biochemically fractionated as described previously (9). Synaptosomal membrane fractions were lysed in NP-40 lysis buffer (0.5% NP-40, 50 mM Tris-HCl [pH 7.5], 50 mM NaCl, 1× complete Mini protease inhibitor cocktail (Roche), 1× PHOSSTOP phosphatase inhibitor cocktail (Roche)) and protein was quantified using the BCA assay (Pierce). Protein lysates were loaded (90 μg/lane) onto SDS-PAGE gels and transferred to PVDF membranes for immunoblotting. Membranes were probed with antibodies against phospho-CSF-1R Y721 (1:1000; Cell Signaling Technology), CSF-1R (1:1000; Santa Cruz Biotechnology), or GAPDH (1:1000; Cell Signaling Technology) and detected using HRP-conjugated anti-rabbit (Jackson Immunoresearch) antibodies using chemiluminescence detection (Millipore). Bands from western blots were quantified in the dynamic range using the Gel analysis module in IMAGEJ software.

Primary bone marrow derived macrophages (BMDMs) were cultured in the absence of CSF-1 for 12 hours prior to stimulation with CSF-1 (10 ng/ml) for the time points indicated in the presence or absence of 67 nM BLZ945. Whole protein lysates were isolated with NP40 lysis buffer and detected by western blot as described above.

### Preparation of Single Cell Suspensions And Flow Cytometry

For investigation of brain macrophage populations by flow cytometric analysis or sorting, the tumor was digested to a single cell suspension by incubation with 5 ml of papain digestion solution (0.94 mg/ml papain [Worthington], 0.48 mM EDTA, 0.18 mg/ml N-Acetyl-L-cysteine [Sigma], 0.06 mg/ml DNase I [Sigma], diluted in Earl's Balanced Salt Solution and allowed to activate at room temperature for at least 30 minutes). Following digestion, the enzyme was inactivated by the addition of 2 ml of 0.71 mg/ml ovomucoid (Worthington). The cell suspension was then passed through a 40 μm mesh to remove undigested tissue, washed with FACS buffer (1% IgG Free BSA in PBS [Jackson Immunoresearch]), and centrifuged at a low speed of 750 rpm (Sorvall Legend RT), to remove debris and obtain the cell pellet.

As many immune cell epitopes are papain-sensitive, for investigation of immune cell infiltration by flow cytometric analysis, tumors were digested to a single cell suspension by incubation for 10 minutes at 37° C. with 5 mL of 1.5 mg/ml collagenase III (Worthington) and 0.06 mg/mL DNase I in 1× Hanks Balanced Salt Solution (HBSS) with calcium and magnesium. The cell suspension was then washed with PBS and passed through a 40 μm mesh to remove undigested tissue. To remove myelin debris, the cell pellet was resuspended at room temperature in 15 ml of 25% Percoll prepared from stock isotonic Percoll (90% Percoll [Sigma], 10% 10× HBSS), and then spun for 15 minutes at 1500 rpm (Sorvall Legend RT) with accelerator and brake set to 1. The cell pellet was then washed with lx HBSS prior to being resuspended in FACS buffer.

After counting, cells were incubated with 1 μl of Fc Block for every million cells for at least 15 minutes at 4° C. Cells were then stained with the appropriate antibodies for 10 minutes at 4° C., washed with FACS buffer, and resuspended in FACS buffer containing DAPI (5 mg/ml diluted 1:5000) for live/dead cell exclusion. Antibodies used for flow cytometry are listed in Table 7.

For analysis, samples were run on a BD LSR II (Becton Dickstein), and all subsequent compensation and gating performed with FLOWJO analysis software (TreeStar). For sorting, samples were run on a BD FACSAria (Becton Dickstein) cell sorter and cells were collected into FACS buffer. Cells were then centrifuged and resuspended in 500 μl Trizol (Invitrogen) before snap freezing in liquid nitrogen and storage at −80° C.

### Derivation of Mouse Primary Glioma Cultures, Neurospheres And Glioma Cell Lines

Macrodissected tumors were digested to a single cell suspension by incubation for 8-12 minutes at 37° C. as described above. The cell suspension was washed with Neural Stem Cell (NSC) Basal Media (Stem Cell Technologies), and centrifuged at low speed (750 rpm Sorvall Legend RT), to remove debris. To derive mouse primary glioma cultures, the cell pellet was resuspended in DMEM containing 10% FBS (Gibco). These primary cultures were used at early passage (P2-P3), and contain a mixture of different cell types found in gliomas including tumor cells, macrophages, and astrocytes as determined by immunofluorescence staining. Primary glioma cultures were grown for 24 hours on poly-L-lysine coated coverslips (BD Biocoat). Cells were then fixed with 4% PFA in 0.1M phosphate buffer overnight at 4° C., permeabilized with 0.1% Triton-X for 5 minutes and blocked with 0.5% PNB for at least one hour. The presence of macrophages, tumor cells and astrocytes were examined by immunofluorescent staining of CD11b (1:200), Nestin (1:500) and GFAP (1:1000), respectively (Table 6).

For neurosphere formation the cell pellet was resuspended in neurosphere media consisting of mouse NSC Basal Media, NSC proliferation supplements, 10 ng/ml EGF, 20 ng/ml basic-FGF and 1 mg/ml Heparin (Stem Cell Technologies). Fresh media was added every 72 hours for 2 weeks. Primary neurospheres were collected, mechanically disaggregated to a single cell suspension and propagated by serial passaging. To generate glioma cell lines, secondary neurospheres were dissociated to single cell suspensions and cultivated in DMEM+10% FBS as a monolayer (10). Multiple glioma cell lines were derived from independent mice, denoted GBM1-4 herein. Glioma cells were infected with a pBabe-H2B-mCherry construct as described previously (11).

### Isolation of Bone Marrow-Derived Macrophages (BMDMs)

For bone marrow isolation, followed by macrophage derivation, C57BL/6 WT, C57BL/6 β-actin-GFP or Nestin-Tv-a; Ink4a/Arf^{-/-} mice were anesthetized with Avertin (Sigma) and then sacrificed via cervical dislocation. Femurs and tibiae were harvested under sterile conditions from both legs and flushed. The marrow was passed through a 40 μm strainer and cultured in 30 ml TEFLON bags (PermaLife PL-30) with 10 ng/ml recombinant mouse CSF-1 (R&D Systems). Bone marrow cells were cultured in TEFLON bags for 7 days, with fresh CSF-1-containing media replacing old media every other day to induce macrophage differentiation.

### Additional Cell Lines

U-87 MG (HTB-14) glioma and CRL-2467 microglia cell lines were purchased from ATCC. The U-87 MG cell line was cultured in DMEM+10% FBS. The CRL-2467 cell line was cultured in DMEM+10% FBS with 30 ng/ml recombinant mouse CSF-1.

### Glioma Cell-Conditioned Media (GCM) Experiments

Media that had been conditioned by glioma tumor cell lines grown in serum free media for 24 hours was passed through 0.22 μm filters to remove cellular debris, and is referred to herein as glioma cell-conditioned media (GCM). GCM was used to stimulate differentiated C57BL/6 WT or β-actin-GFP⁺ BMDMs. Control macrophages received fresh media containing 10% FBS and 10 ng/ml recombinant mouse CSF-1. When indicated, differentiated BMDMs were cultivated in GCM containing either DMSO as vehicle, or 67 nM BLZ945, 670 nM BLZ945, or in regular media containing 10 ng/ml mouse recombinant CSF-1 and 10 ng/ml IL-4 (R&D Systems) for 24 hours or 48 hours prior to experimental analysis.

### Analysis of Mrc1/CD206 Expression By Flow Cytometry

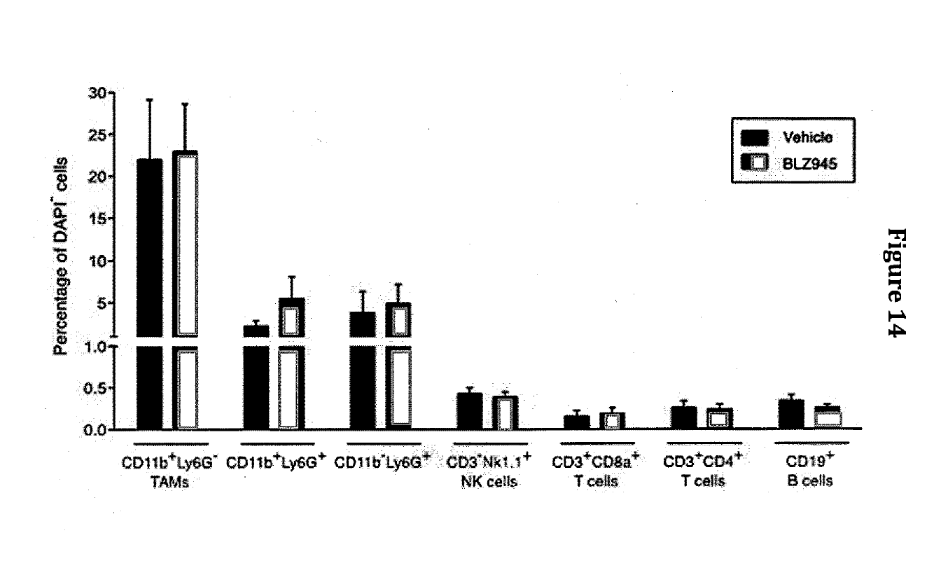

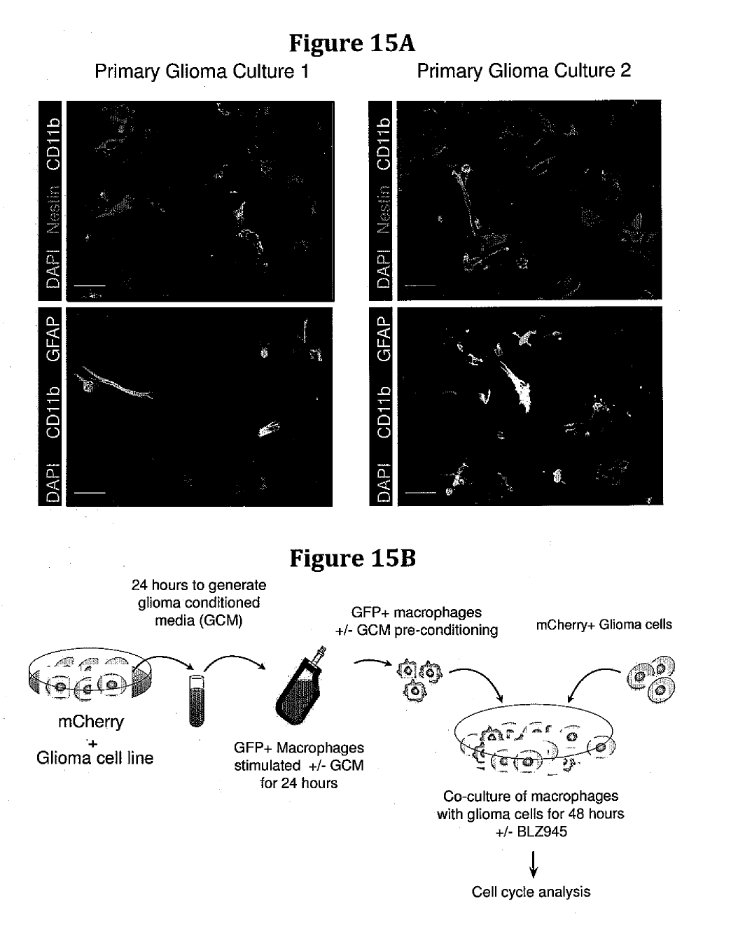

For mouse primary glioma cultures (containing a mixed population of tumor cells, TAMs, astrocytes etc.; see FIG. 15), 1×10⁶ cells were cultivated in DMEM+10% FBS in the presence of the CSF-1R inhibitor or DMSO as vehicle (n=6 independently isolated cultures). For BMDMs, 1×10⁶ cells were cultivated in DMEM supplemented with recombinant mouse CSF-1 or GCM in the presence of the CSF-1R inhibitor or DMSO as vehicle. After 48 hours, cells were scraped and washed with FACS buffer. Cells were counted and incubated with 1 μl of Fc Block (BD Pharmingen) per 10⁶ cells for at least 15 minutes at 4° C. Cells were then stained with CD45 and CD11b antibodies (Table 7) for 10 minutes at 4° C. and washed with FACS buffer. Cells were fixed and permeabilized using the BD Cytofix/Cytoperm™ kit (BD Biosciences) according to the manufacturer's instructions. Subsequently cells were stained with anti-CD206 antibody (Table 7). For analysis, samples were run on a BD LSR II (Becton Dickstein), and all subsequent compensation and gating performed with FLOWJO analysis software (TreeStar).

### Cell Cycle Analysis

Control or GCM pre-stimulated macrophages derived from β-actin-GFP⁺ mice were co-cultured in a 1:1 ratio with 1×10⁵ serum starved mCherry-positive glioma cells (from the cell lines derived above) for 48 hours in the presence of 670 nM BLZ945 or DMSO as vehicle. Following collection of trypsinized co-cultured cells, wells were rinsed in additional media and this volume was collected to ensure harvesting of all macrophages, which adhered tightly to cell culture dishes. Samples were then washed once with FACS buffer, followed by incubation for 10 minutes at room temperature in permeabilizing buffer (10 mM PIPES, 0.1 M NaCl, 2 mM MgCl2, 0.1% Triton X-100, pH 6.8) containing 0.1 mg DAPI (Invitrogen). After acquisition on an LSR II flow cytometer (BD) using a UV laser (350-360 nm), cell cycle status of glioma tumor cells was analyzed using the Flow Jo Dean-Jett-Fox program for cell cycle analysis.

### Proliferation Assays

Cell growth rate was determined using the MTT cell proliferation kit (Roche). Briefly, cells were plated in triplicate in 96-well plates (1×10³ cells/well for glioma cell lines and 5×10³ cells/well for BMDM and CRL-2467 cells) in the presence or absence of 6.7-6700 nM of BLZ945. Media was changed every 48 hours. BMDM and CRL-2467 cells were supplemented with 10 ng/ml and 30 ng/ml recombinant mouse CSF-1 respectively unless otherwise indicated. Ten μl of MTT labeling reagent was added to each well and then incubated for 4 hours at 37° C., followed by the addition of 100 μl MTT solubilization reagent overnight. The mixture was gently resuspended and absorbance was measured at 595 nm and 750 nm on a SPECTRAMAX 340 pc plate reader (Molecular Devices).

### Secondary Neurosphere Formation Assay

Primary neurospheres were disaggregated to a single cell suspension and 5×10³ cells were plated in a 6 well plate in neurosphere media in the presence of the CSF-1R inhibitor or DMSO as vehicle. Media was changed every 48 hours. Secondary neurosphere formation was assayed by counting the number of neurospheres obtained after 2 weeks.

RNA Isolation, cDNA Synthesis And Quantitative Real Time PCR

RNA was isolated with TRIZOL, DNase treated, and 0.5 μg of RNA was used for cDNA synthesis. TAQMAN probes (Applied Biosystems) for Cd11b (Mm00434455_m1), Cd68 (Mm03047343_m1), Csf-1 (Mm00432688_m1), Csf-1r (Mm00432689_m1), 1134 (Mm00712774_m1), Mrc1 (Mm00485148_m1), and Tv-a (custom), were used for qPCR. Assays were run in triplicate and expression was normalized to ubiquitin C (Mm01201237_m1) for each sample.

### Microarrays And Gene Expression Profiling

RNA was isolated using TRIZOL and the quality was assessed by running on an Agilent Bioanalyzer. 75 ng of total RNA was reverse transcribed and labeled using the GENECHIP 3′ IVT Express Kit (Affymetrix). The resulting cRNA was hybridized to Affymetrix MOE 430A 2.0 chips. Raw expression data were analyzed using GCOS 1.4 (Affymetrix). Data were normalized to a target intensity of 500 to account for differences in global chip intensity.

### Microarray Analysis

All bioinformatics analyses were completed in R using the Bioconductor suite of packages. Expression values were computed using the robust multi-array average (RMA) method and then quantile normalized in the ‘affy’ package (12, 13). The ‘limma’ package (14) was used to identify differentially expressed genes between the vehicle and the CSF-1R inhibitor-treated samples. Differential expression was considered significant at a fold change of +/−2 with a false discovery rate of 10%. Gene set enrichment analysis (GSEA) was used as described previously (15). For subsequent analysis and comparison to human datasets, mouse expression values were mean centered across all samples.

### Lasso Regression Method For Gene Signature Identification

Mouse expression data was normalized and mean centered as described above. Differentially expressed genes were used for further analysis. A logistic regression model with lasso constraints was trained to differentiate between Vehicle and CSF-1R inhibitor-treated samples using the ‘glmnet’ package (16) by setting the ‘family’ parameter to ‘binomial’ in the glmnet function. The regularization parameter for lasso regression was chosen by 4-fold cross validation.

### Patient Datasets

TCGA expression data was downloaded from the TCGA data portal and all clinical data was downloaded from the data portal (17). Clinical and expression data for the Rembrandt data set was downloaded from the NCI website. The Freije (GSE4412), Murat (GSE7696), and Phillips (GSE4271) datasets were downloaded from the NCBI website (18-20). For the Freije datasets, only samples that were run on the HGU133A platform were considered as samples on the HGU133B platform contained minimal overlap with the remaining datasets. Datasets were individually processed and normalized as described above. Within each dataset, genes were mean centered across patients.

### Subtyping of Non TCGA Patients

To investigate subtype specific survival differences in all publically available datasets, a subtype classification described previously (21) was utilized to train a support vector machine (SVM). The 840 genes used by Verhaak and colleagues for the ClacNc analysis were used to subset the dataset (21). Subsequently, data sets were subsetted for genes that were called present across all patient data sets described above. The remaining 776 genes were used to train a multiclass SVM on the Core samples from the TCGA dataset. The SVM was completed using a Gaussian radial basis kernel function using the ‘kernlab’ package (22). This SVM was then used to predict the subtype of the remainder of the TCGA patients and public datasets.

### Patient Classification

An SVM on mouse expression data was trained to classify patients into “Vehicle-like” classification or “Treatment-like (BLZ945-like)” classification. Patient expression data was subsetted for common genes across all data sets and genes that have known mouse homologues. Similarly, mouse expression data was subsetted for genes with human homologues that were common across all patient samples. Subsequently, mouse data was subsetted for differentially expressed genes identified using the ‘limma’ package. Human data was subsetted for the human homologues of these differentially expressed genes. This led to a feature reduction from 257 differentially expressed genes to 206 differentially expressed genes with known human homologues across all patient datasets. The ‘kernlab’ package was then used to train an SVM on the mouse expression data using a vanilla kernel function. This SVM was then used to predict patients into either “Vehicle-like” class or “Treatment-like” class.

A similar approach was used to classify patients with a lasso logistic regression model. The restriction to genes with human-mouse homologs in the patient and mouse data was identical to that described above. Instead of using the ‘kernlab’ package, a lasso logistic regression model was trained using the ‘glmnet’ package. This model was then used to predict patient classification into either “Vehicle-like” class or “Treatment-like” class. G-CIMP patient status was determined by hierarchical clustering of patient methylation data (23) as described below.

### Stratification of Patients By G-CIMP Status

It was determined whether the survival advantage offered by the “Treatment-like” treatment signature was potentially due to an enrichment of Glioma CpG Island Methylator Phenotype (G-CIMP) patients, which have previously been shown to be associated with improved overall survival (23). Of the 453 GBMs analyzed from the TCGA dataset, 263 also had genomic methylation data and were classified into the methylation clusters as described previously (23). Of the 21 G-CIMP patients, 20 (95%) were classified into the “Treatment-like” classification, showing a strong enrichment of CSF-1R inhibitor-treated samples in the G-CIMP patients. Despite this enrichment, survival analysis of Proneural patients known to be G-CIMP negative (67/133 total Proneural patients) revealed that the “Treatment-like” classification group still showed an increase in survival of ˜10.8 months (P=0.014). Moreover, cox proportional hazard models demonstrated that the increase in survival demonstrated by “Treatment-like” classification was not dependent upon G-CIMP patients. The hazard ratio associated with the gene signature was significant with and without G-CIMP patients (Table 4). Also, the hazard ratio for G-CIMP strata was not significant when the gene signature was also considered in a mixed model (Table 4). Thus, although the G-CIMP patients are clearly enriched for mock “Treatment-like” classification samples, the survival benefit offered by this classification is not dependent upon G-CIMP status.

### Survival Analysis

Survival analysis was completed using the ‘survival’ package in R (24). Hazard ratios were determined utilizing the ‘coxph’ function from the ‘survival’ package. Patients were stratified based on the probability of the lasso logistic regression classification, G-CIMP status, or both as indicated. P values were generated using Wald's test.

### Plots For Patient Analyses

All Kaplan-Meier survival curves, heatmaps and volcano plots were generated in R v 2.14.1 using the ‘gplots’ package (25). Hazard ratio forest plots were generated in GraphPad Prism Pro5.

### Data Presentation And Statistical Analysis

Data are presented as means with their respective standard error (SEM) or as statistical scatter plots using GraphPad Prism Pro5. Numeric data were analyzed by unpaired two-tailed Student's t-test unless otherwise noted. For survival curves, P values were obtained using the Log Rank (Mantel-Cox) test, and Fisher's exact test was used for histological tumor grading. P<0.05 was considered as statistically significant.

### EXAMPLE 2

### CSF-1R Inhibition Alters Macrophage Polarization And Blocks Gliomagenesis

### Mouse Model of Gliomagenesis

The experiments described below used the RCAS-PDGF-B/Nestin-Tv-a;Ink4a/Arf−/− mouse model of gliomagenesis (5, 6), hereafter referred to as PDGF-driven gliomas (PDG). This model is ideal for preclinical studies as it recapitulates all pathological features of human GBM in an immunocompetent setting.

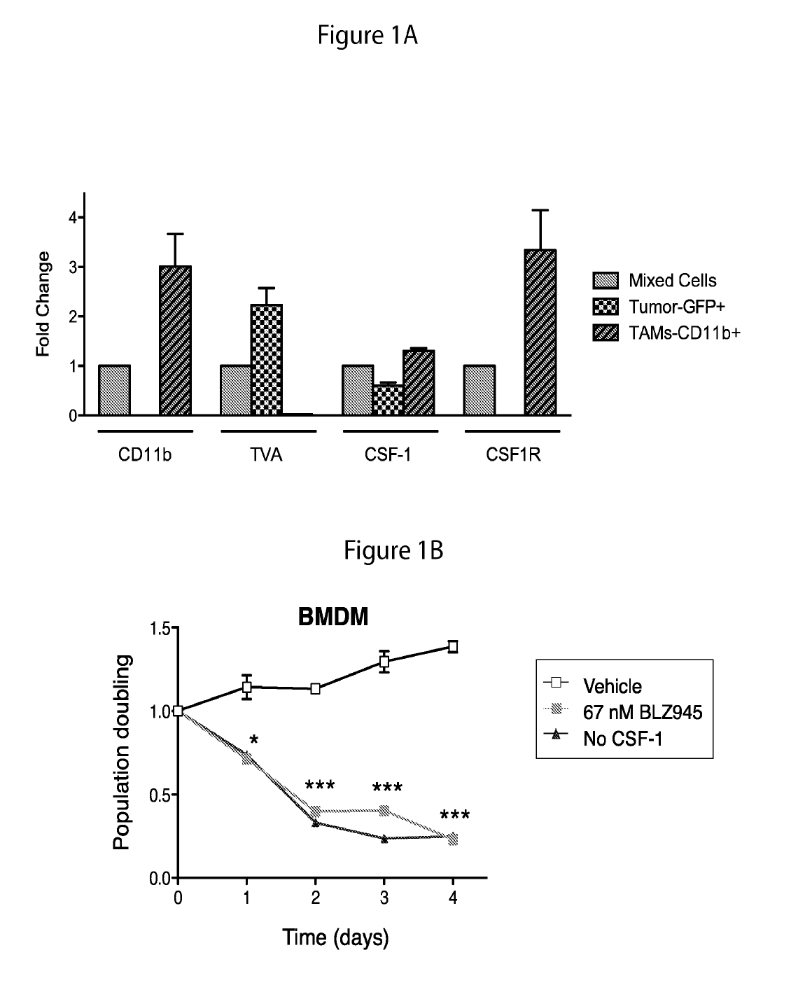

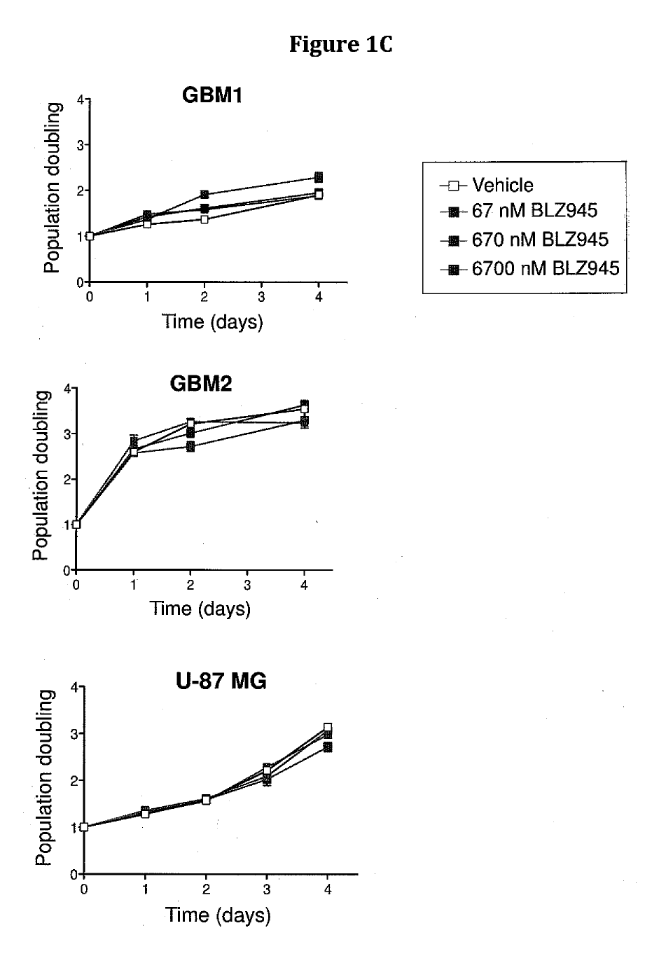

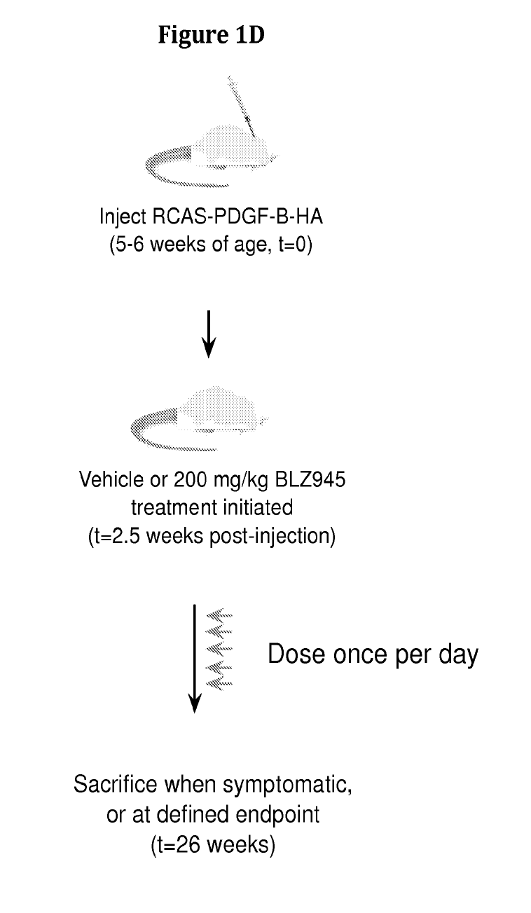

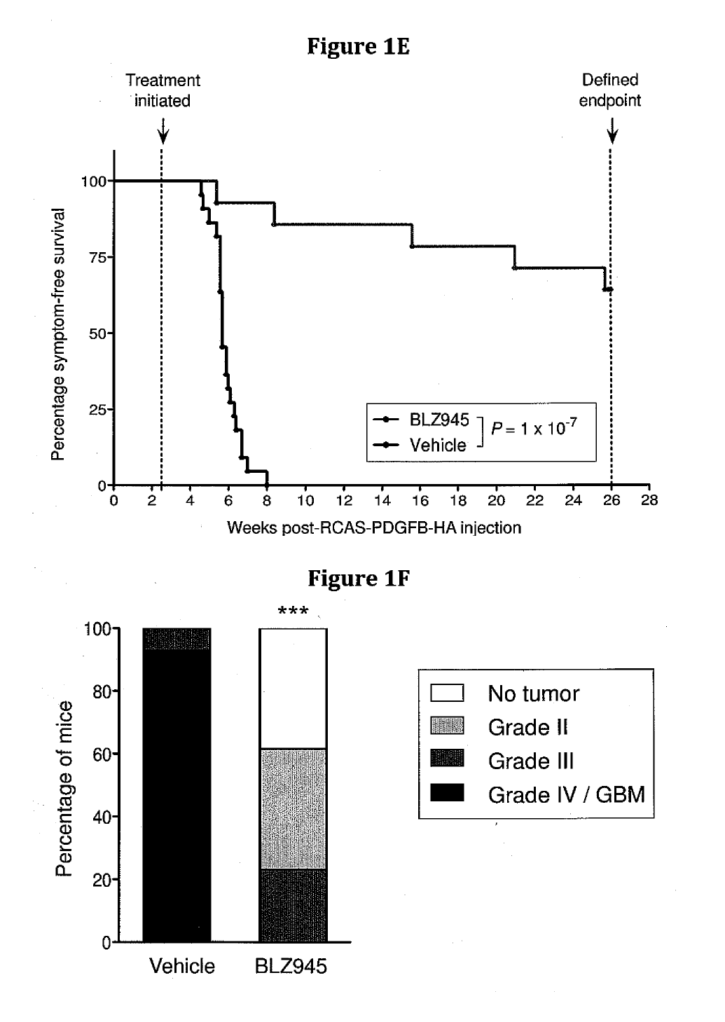

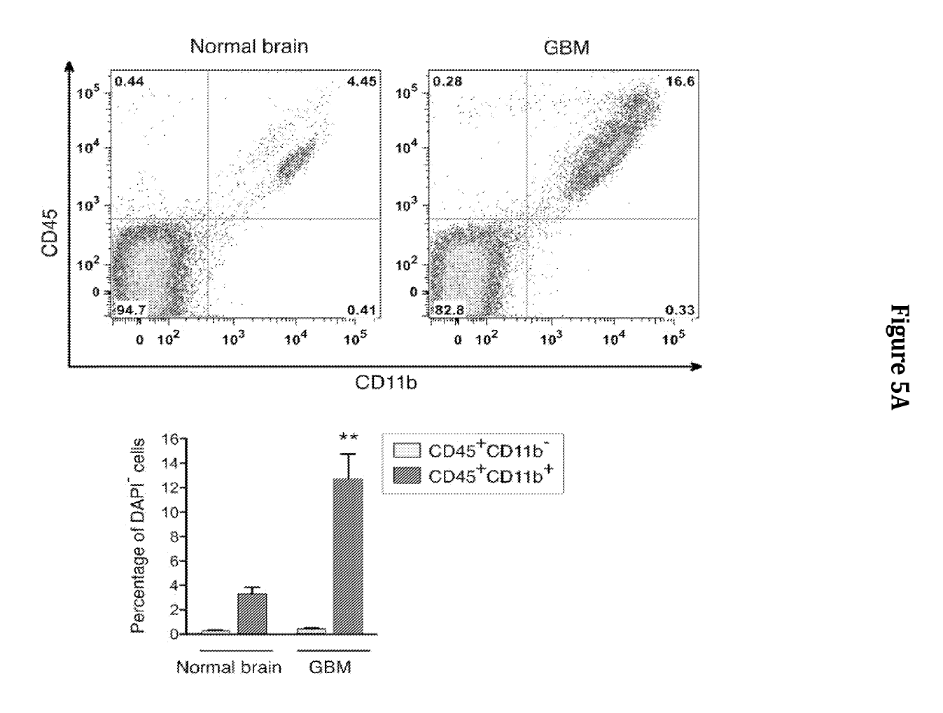

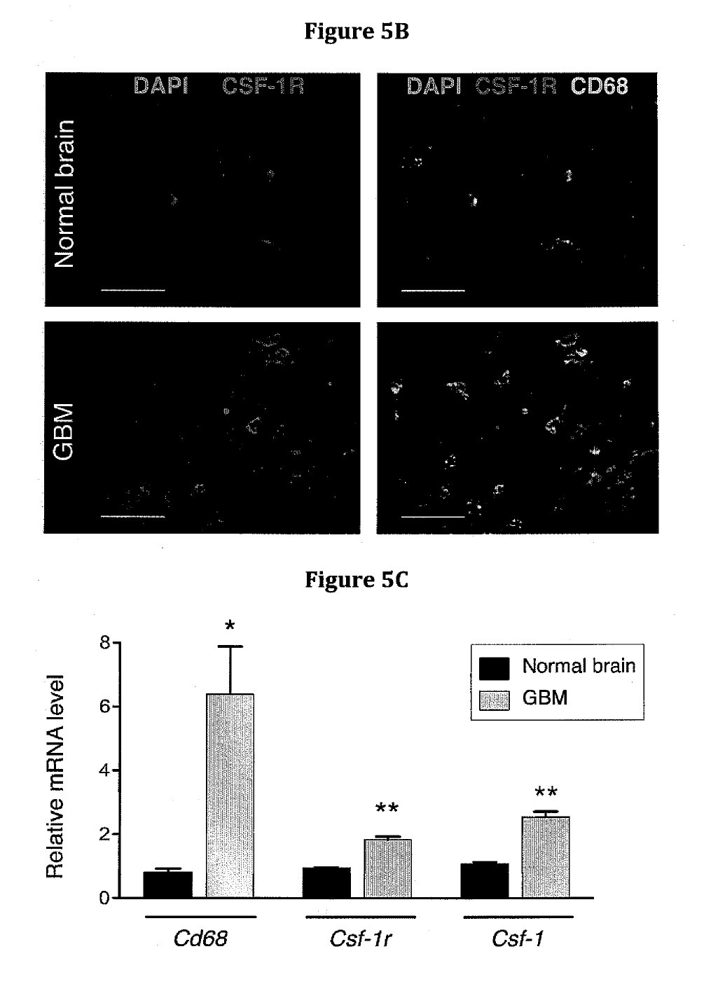

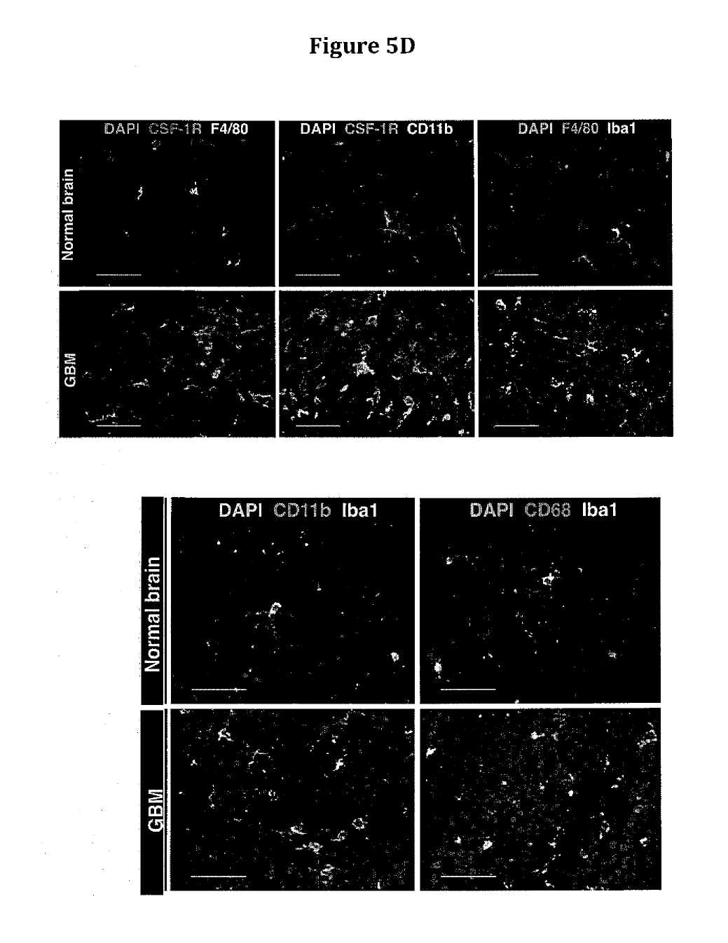

It was first investigated if PDG tumors showed increased macrophage accumulation as reported in human gliomas. Comparison of normal brain versus GBM via flow cytometry demonstrated elevated CD11b+ myeloid cells/macrophages, representing the vast majority of leukocytes (FIG. 5). These data were confirmed by immunostaining of tissue sections for different macrophage markers including CD68 and Iba1. Similarly, mRNA expression data from whole tumors revealed increased Cd68, Csf-1 and Csf-1r. Expression analysis of FACS-purified tumor cells and CD11b+Gr-1− macrophages from gliomas showed Csf-1 was expressed by both cell types, while Csf-1r was only amplified in the macrophages (FIG. 1A). This was verified by immunostaining, showing all CSF-1R+ cells co-stained with CD68 both in normal brain and GBM (FIG. 5). Thus it is concluded that any phenotypes observed by targeting the CSF-1R pathway in this GBM model are macrophage dependent.

### CSF-1R Inhibitor

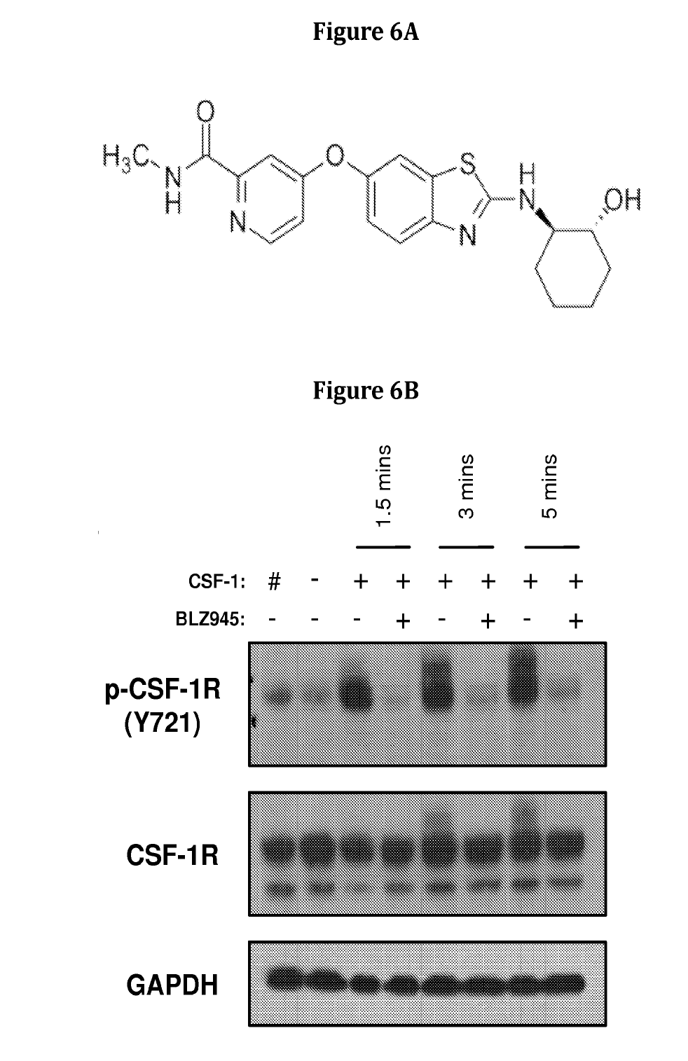

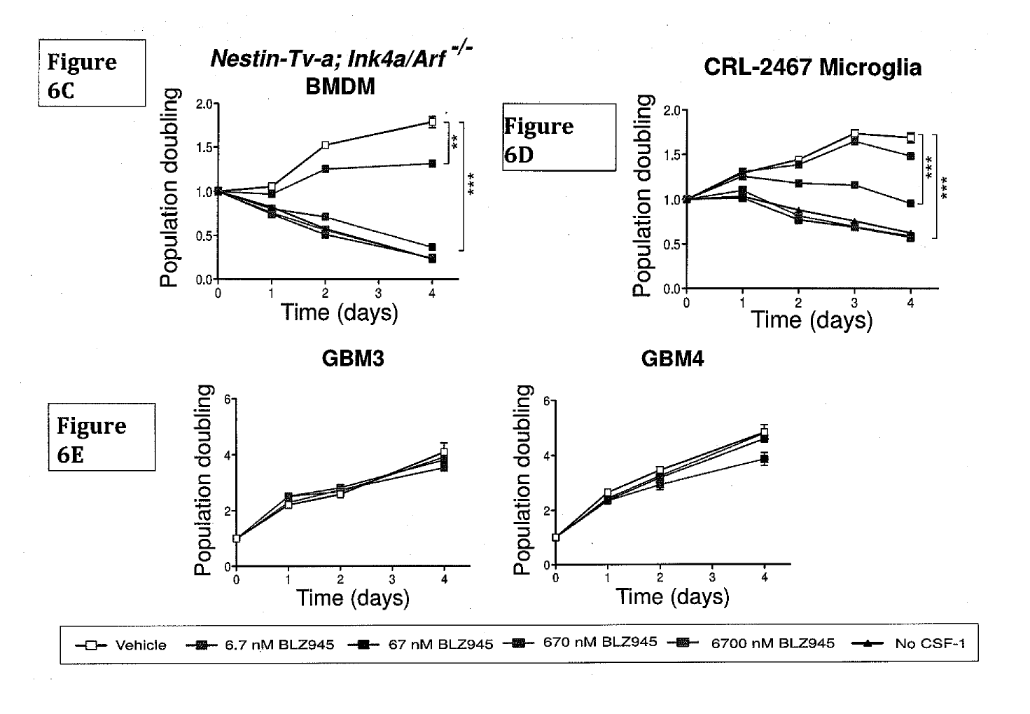

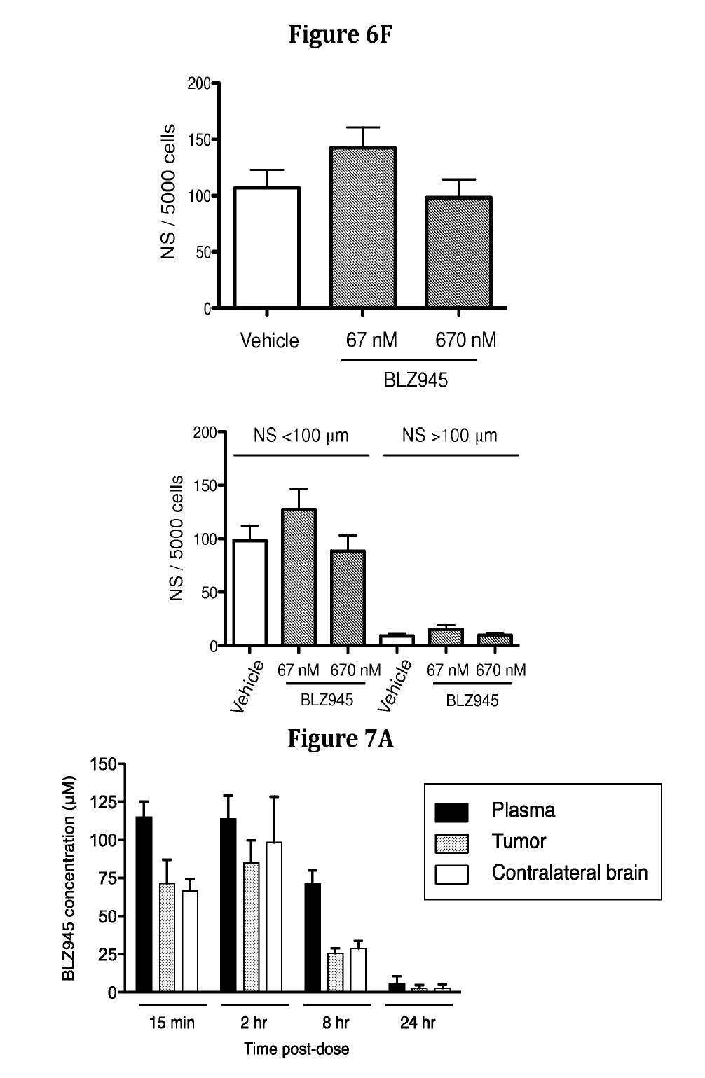

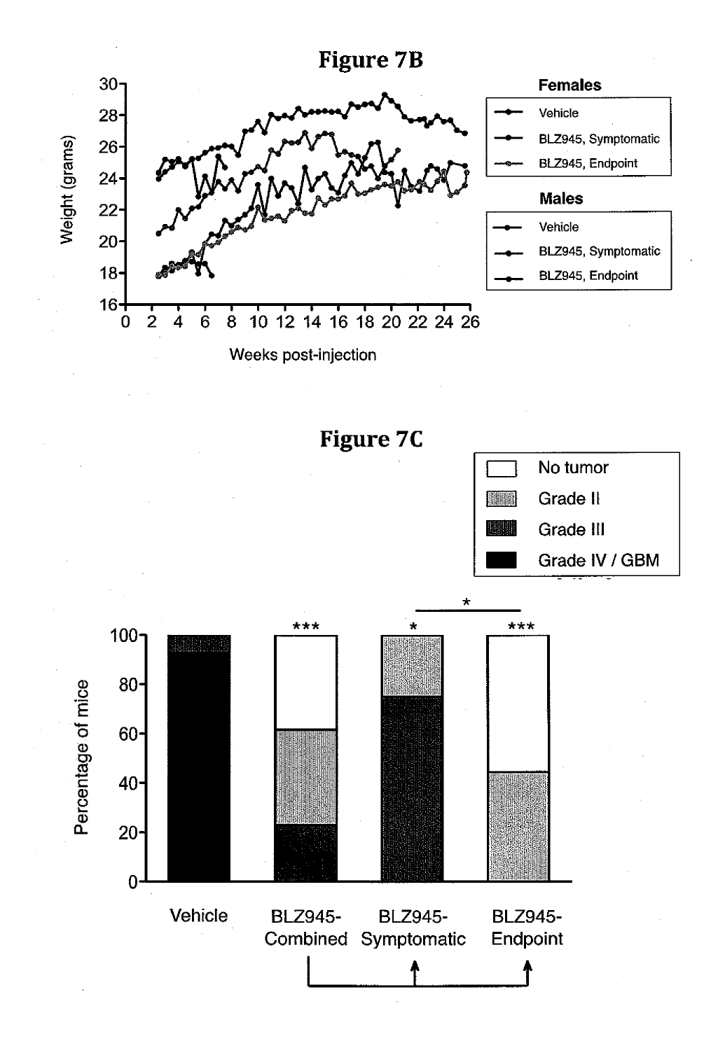

The CSF-1R inhibitor used herein is a potent, highly selective, brain penetrant CSF-1R inhibitor that blocks CSF-1R phosphorylation and kinase activity (FIGS. 6 and 7A). In biochemical assays, the inhibitor inhibits CSF-1R at 1 nM, and CSF1-dependent cell proliferation at 67 nM. By comparison the biochemical IC50 values for >200 kinases tested, including PDGFRα (the receptor for PDGF-B), were >10 μM, with the exception of cKIT and PDGFRβ (3500 nM and 3300 nM respectively) (data not shown). The inhibitor inhibits CSF-1R phosphorylation and significantly decreases the viability of primary bone marrow-derived macrophages (BMDMs) in culture, similar to CSF-1 withdrawal (FIGS. 1B and 6). The inhibitor also blocked the survival of Ink4a/Arf−/−BMDMs, the genetic background of the PDG model, and reduced viability of the microglia cell line CRL-2467 (FIG. 6). Importantly, concentrations up to 6700 nM BLZ945 had no effect on the proliferation of 4 different tumor cell lines derived from PDG mice, tumor neurosphere formation or U-87 MG human glioma cell proliferation (FIGS. 1C and 6). Inhibition of PDGF signaling has been shown to reduce U-87 MG glioma cell viability, and tumor cells from the PDG model are also sensitive to PDGFR inhibition in vivo and in culture (data not shown). As no effect of the inhibitor on tumor cell viability in monoculture was observed, this strongly argues against any off-target effects on PDGFR signaling. Collectively, these cell culture experiments demonstrate that the biological effects of CSF-1R inhibition are specific to macrophages, with no evident direct effects on tumor cells.

### Therapeutic Potential In Preclinical Trials

The therapeutic potential of the inhibitor was next assessed in preclinical trials using the PDG model. At 2.5 weeks post-tumor initiation, cohorts of mice were treated with either the CSF-1R inhibitor or the vehicle control, and evaluated for symptom-free survival (FIG. 1D). Median survival in the vehicle-treated cohort was 5.7 weeks, with no animals surviving past 8 weeks post-injection. In contrast, 64.3% of the CSF-1R inhibitor-treated cohort was still alive at the trial endpoint of 26 weeks post-injection (FIG. 1E). This endpoint was chosen because mice in the Ink4a/Arf−/−background start developing spontaneous tumors around 30 weeks of age. Over this extended time period, the PDG mice did not exhibit any visible side effects and the CSF-1R inhibitor was well-tolerated (FIG. 7B). Tumor grade was examined in both cohorts of mice; all vehicle-treated mice at end stage had high-grade tumors (FIG. 1F). In contrast, the CSF-1R inhibitor-treated animals had significantly less malignant tumors. This group was then stratified into mice sacrificed as symptomatic during the trial, from those still asymptomatic when sacrificed at the 26-week trial endpoint. In each CSF-1R inhibitor group, there was still a significant decrease in tumor grade compared to the vehicle cohort, and remarkably there were no detectable lesions in 55.6% of the asymptomatic mice at end stage.

### Effects of the CSF-1R Inhibitor on Established Tumors

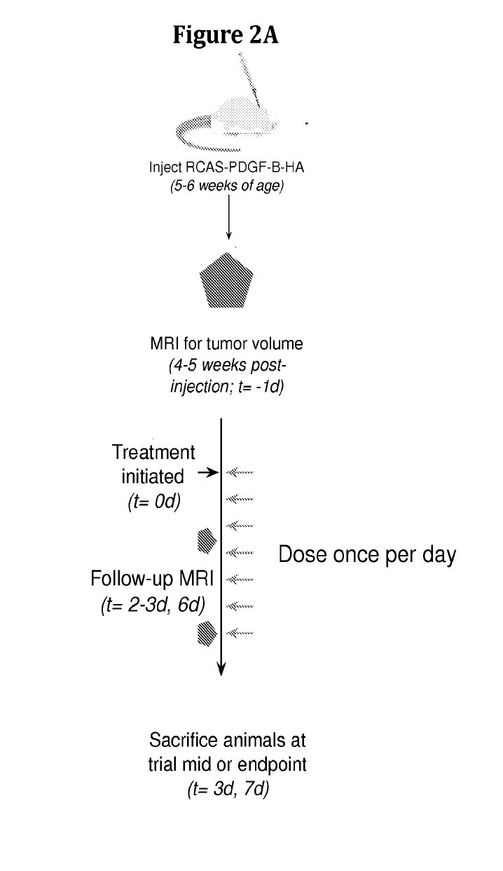

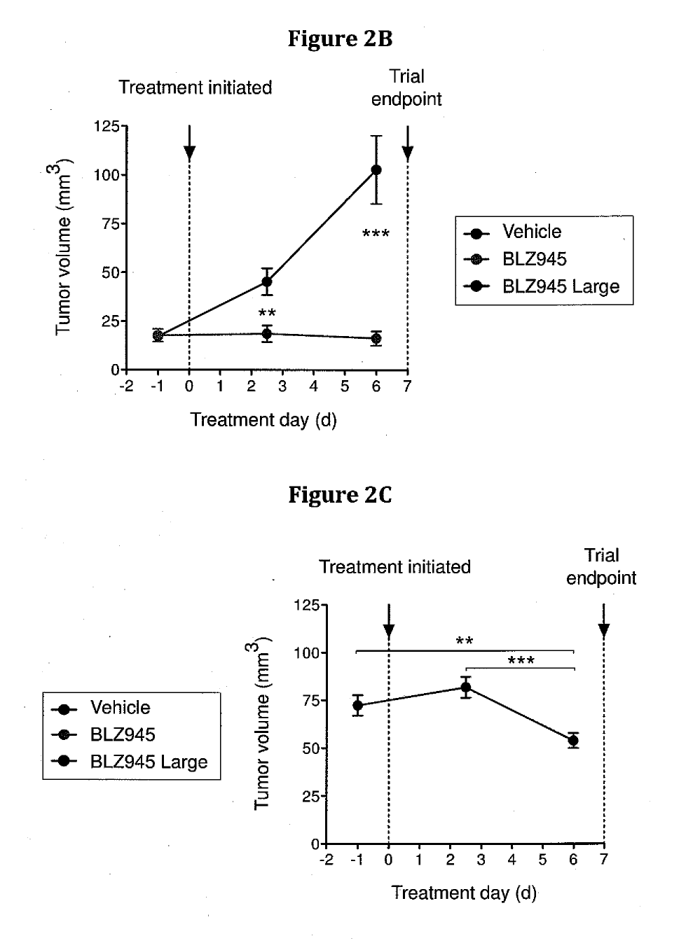

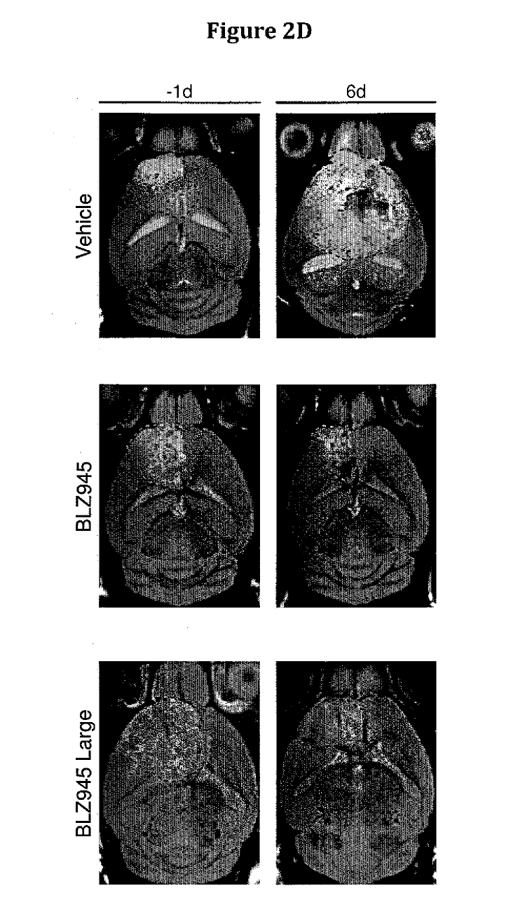

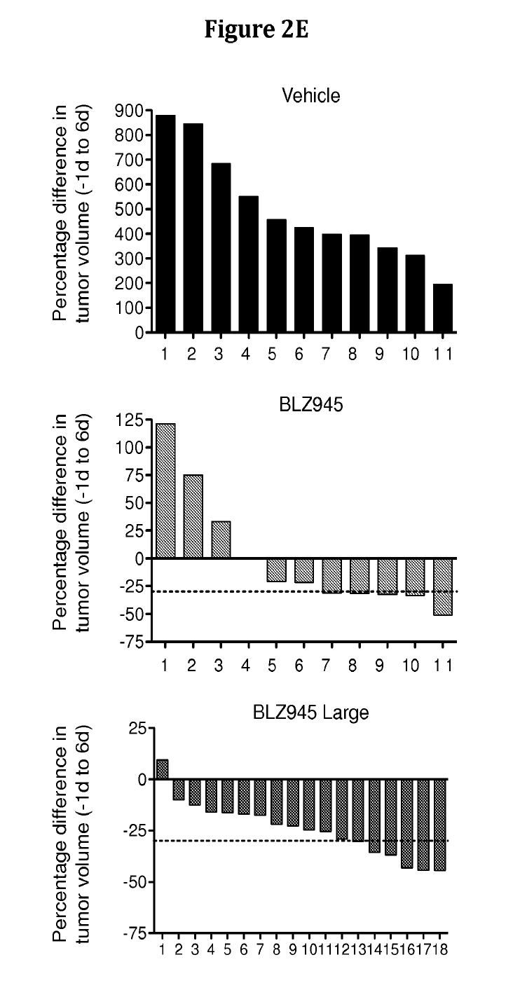

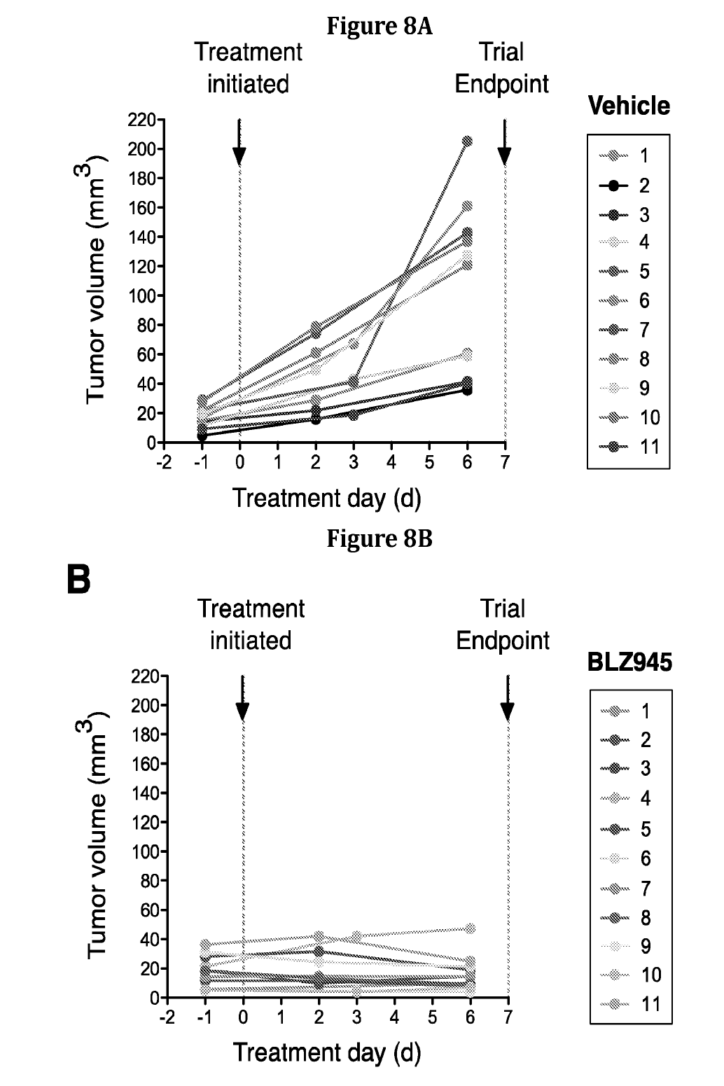

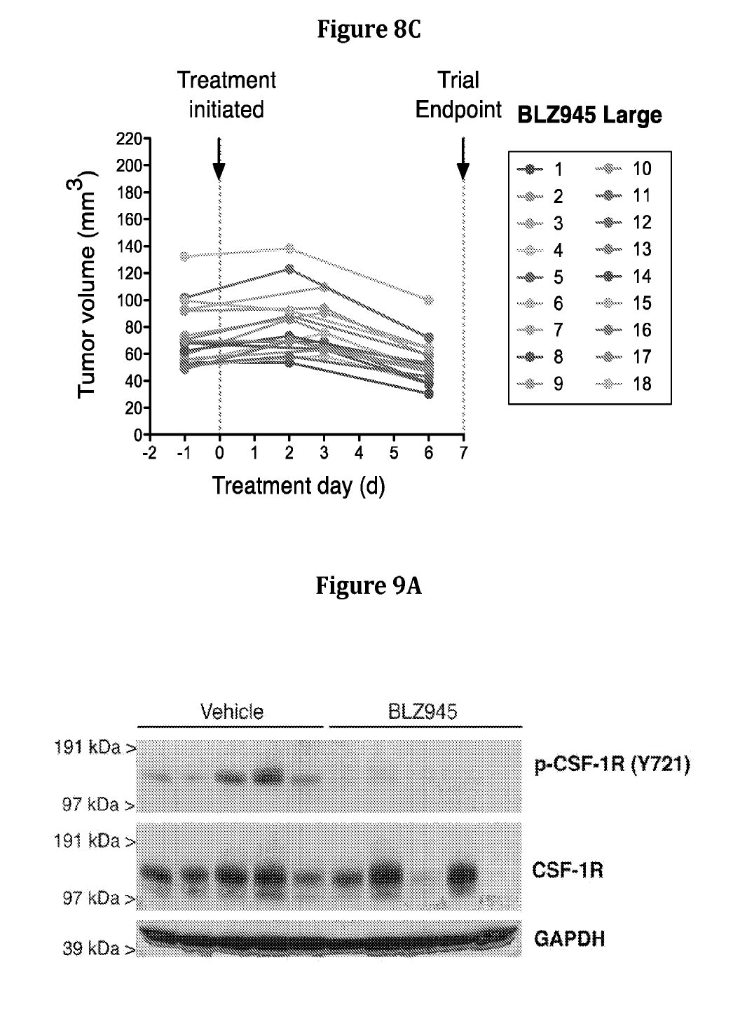

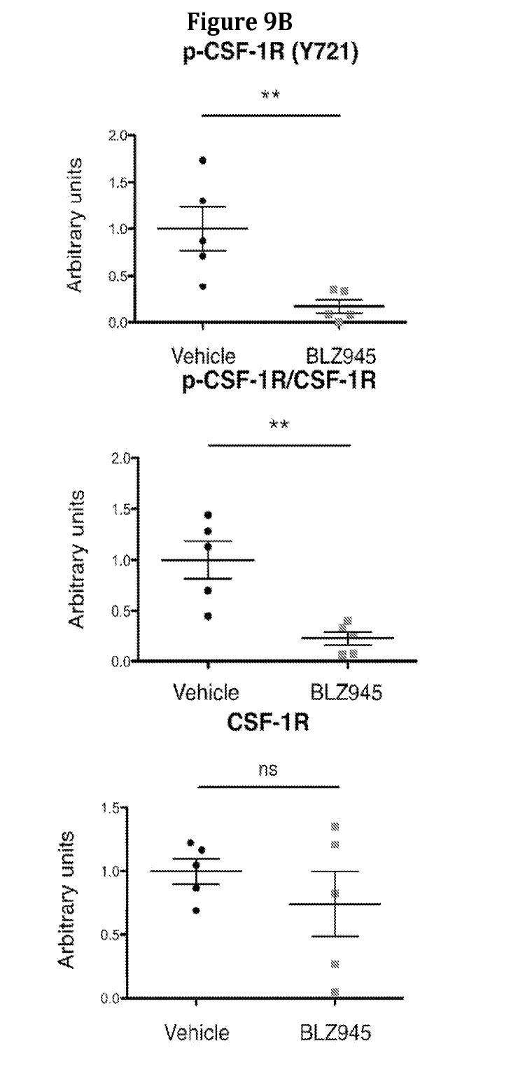

To directly monitor the effects of the CSF-1R inhibitor on established tumors, a short-term 7 day trial incorporating MRI scans to assess initial tumor volume and subsequent growth (FIG. 2) was performed. When PDG mice had a tumor volume of 4.5-40 mm3 they were randomized into CSF-1R inhibitor or vehicle cohorts. In the vehicle group, there was a progressive and substantial increase in tumor growth, ranging from 195-879%. In contrast, treatment with the CSF-1R inhibitor significantly halted tumor growth, with a majority showing either no change or a decrease in tumor volume (FIGS. 2B, 2D, and 8). Given this pronounced effect, a third cohort of mice with tumors >40 mm3 was treated with the CSF-1R inhibitor (henceforth denoted as ‘BLZ945 Large’). Initial tumor volume in this group ranged from 48.7 to 132.3 mm3. A size-matched vehicle cohort was not included for comparison because the mice would not have survived to the trial endpoint. Treatment of large tumors with the CSF-1R inhibitor also showed a striking response (FIGS. 2C, 2D, and 8). Graphing the individual changes in tumor volume in a waterfall plot revealed that 6 of 18 mice had a >30% reduction in tumor volume in this very short time period (FIG. 2E), qualifying as a partial response according to Response Evaluation Criteria in Solid Tumors (RECIST). Inhibition of CSF-1R phosphorylation was confirmed in the CSF-1R inhibitor-treated tumors (FIG. 9).

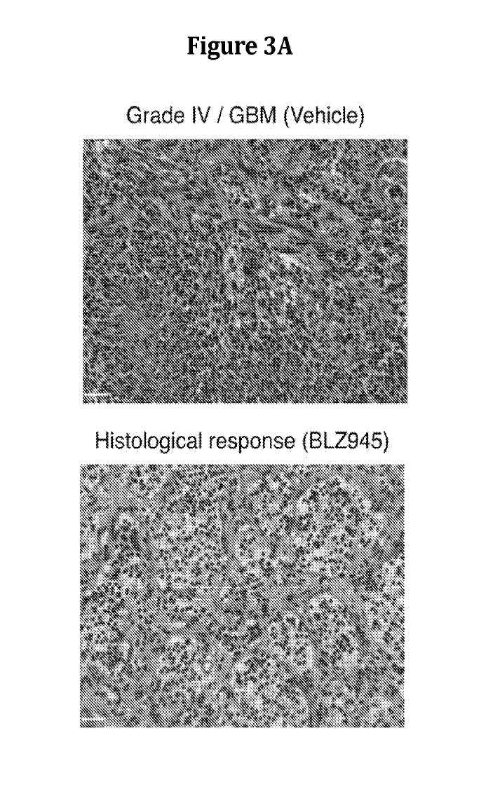

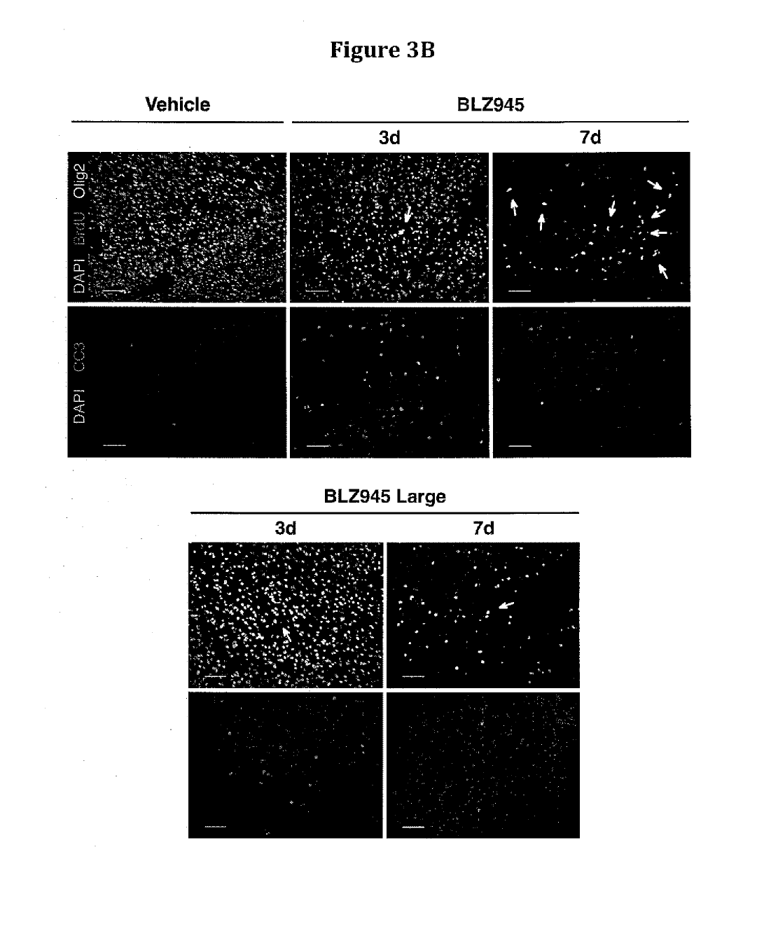

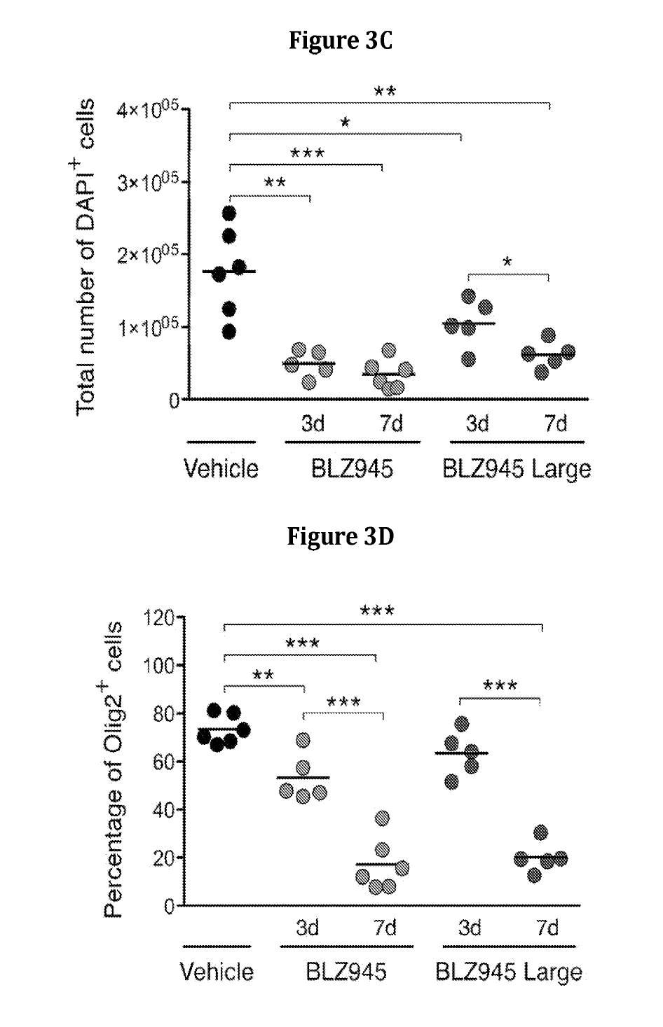

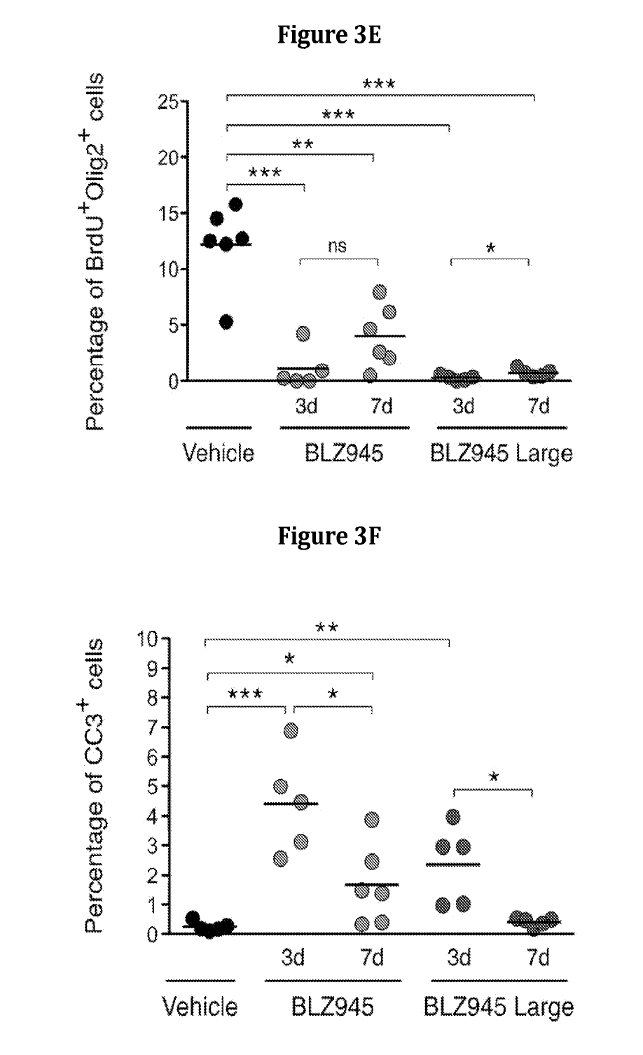

To characterize the response to the CSF-1R inhibitor, tumor grade was scored histologically. While all of the vehicle treated mice had high-grade tumors, with 89% having grade IV GBMs, 100% of the CSF-1R inhibitor-treated mice exhibited a tumor response already evident at d3 (FIGS. 3A-B). This response was characterized by a clear depopulation of tumor cells, with maintenance of the stroma and leukocytic infiltrate. To understand the cellular mechanisms underlying the striking tumor response, how treatment with the CSF-1R inhibitor affected several hallmark capabilities of cancer was investigated (Table 1). All analysis was performed on tissues from the short-term trial, including time points from the midpoint (d3) and endpoint (d7) to capture potentially dynamic changes in response to the CSF-1R inhibitor. Consistent with the histological data, the total number of cells within the region of the original tumor dramatically decreased in both the CSF-1R inhibitor cohorts at all time points tested compared to vehicle controls (FIGS. 3C-D). A progressive reduction in the number of tumor cells, positive for the oligodendrocyte marker Olig2 was observed. By d7, the average density of Olig2⁺ tumor cells was reduced to <20% of total cells in both CSF-1R inhibitor groups (FIG. 3E). Analysis of tumor cell proliferation showed a pronounced decrease in the CSF-1R inhibitor groups at all time points, ranging from 67-98% reduction (FIG. 3F).

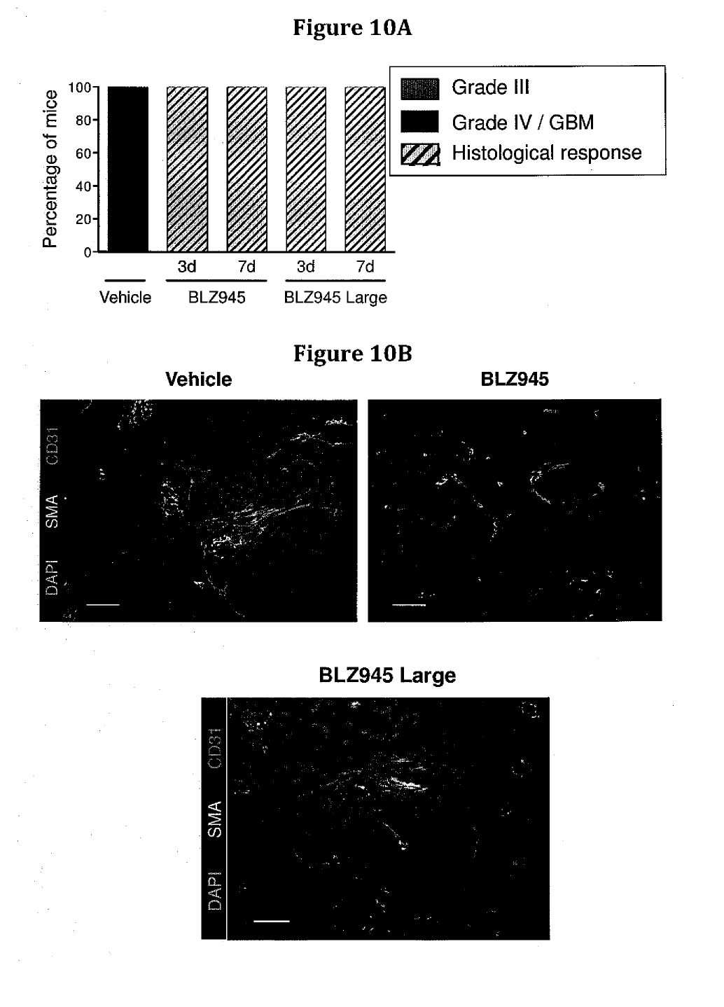

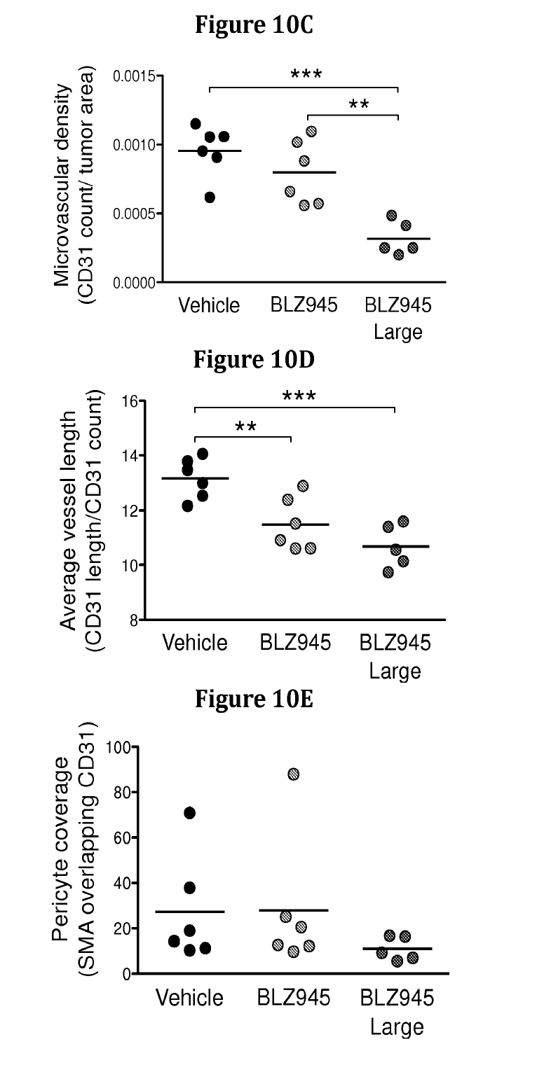

Sustained tumor growth requires the development of an adequate vasculature, and in gliomas, neo-angiogenesis is characteristic of high-grade tumors. Given the striking reduction in tumor proliferation, whether CSF-1R inhibitor treatment affected the vasculature that could indirectly impact tumor growth was investigated. Microvessel density was decreased in the Large tumor group, and the average blood vessel length decreased in both CSF-1R inhibitor treatment groups compared to vehicle (FIG. 10). There were no significant differences in pericyte coverage of the vessels.

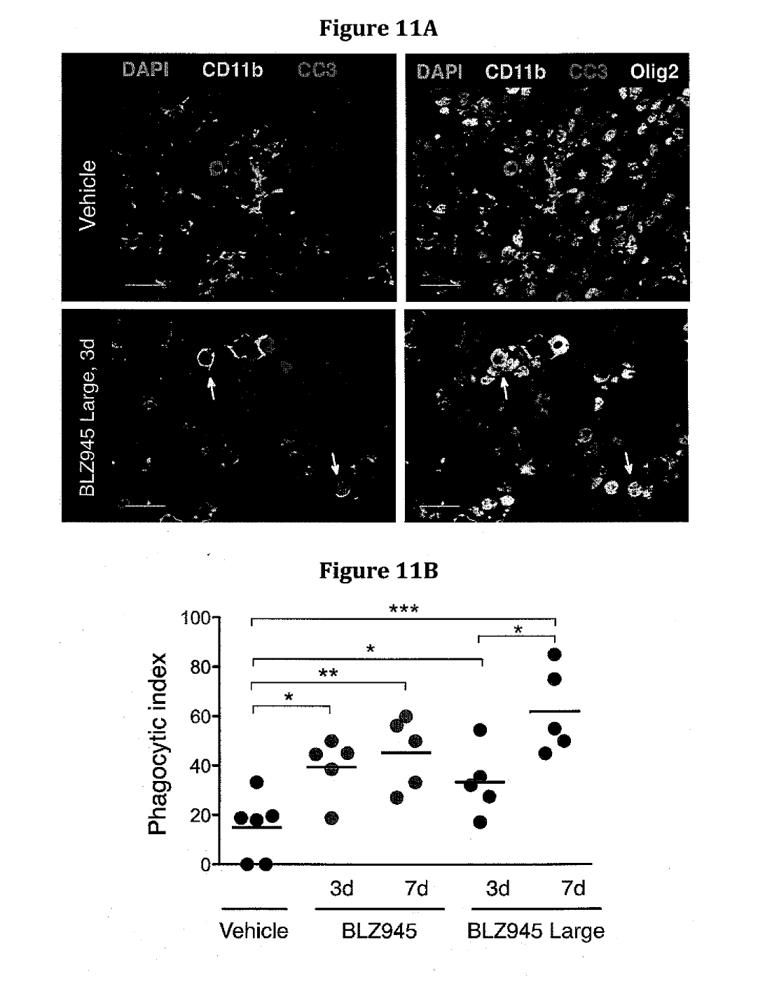

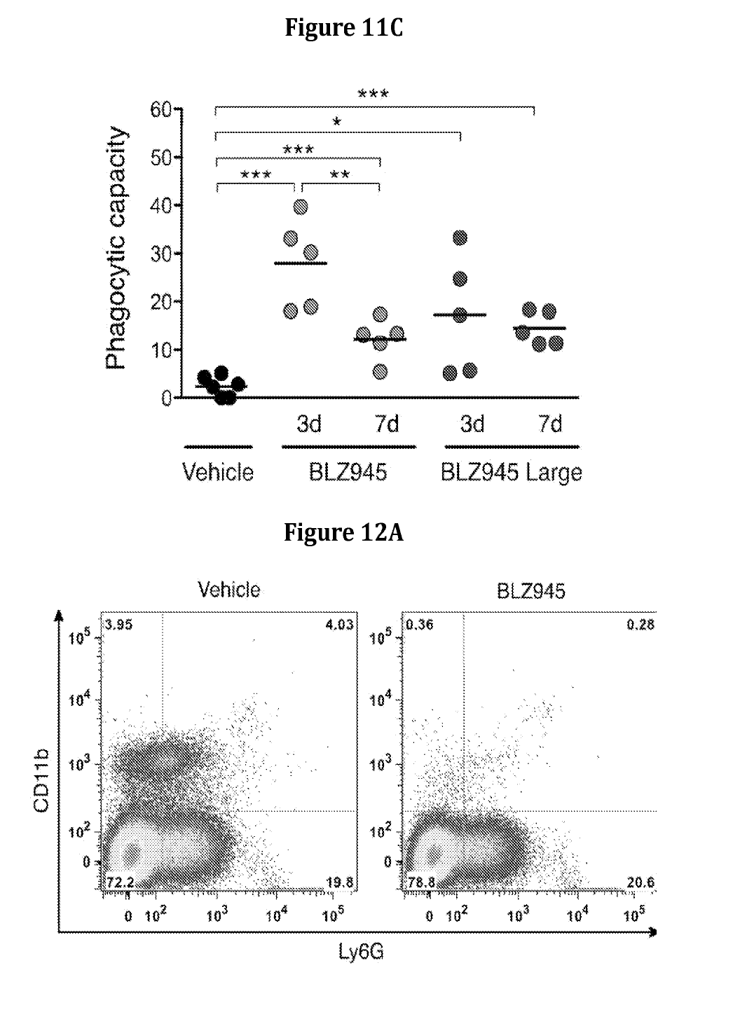

Apoptosis was examined next and a substantial 9-to 17-fold increase on d3 was found (FIGS. 3C, 3G). Apoptotic cells were less prevalent at d7 in both CSF-1R inhibitor cohorts, suggesting the increase in apoptosis is primarily an early response to CSF-1R inhibition. Whether macrophages cleared the dead tumor cells by phagocytosis was investigated. Tissues were co-stained for the macrophage cell surface marker CD11b, tumor cell marker Olig2, and cleaved caspase-3 (CC3) (FIG. 11). The number of Olig2⁺CC3⁺ cells clearly engulfed by CD11b⁺ macrophages increased by 2 to 4-fold in the CSF-1R inhibitor-treated groups. These macrophages also exhibited 5 to 11.5-fold increased phagocytic capacity. Collectively, these analyses demonstrate that inhibition of CSF-1R signaling effectively blocks the growth and malignancy of gliomas through a combined effect on reducing tumor cell proliferation and increasing cell death.

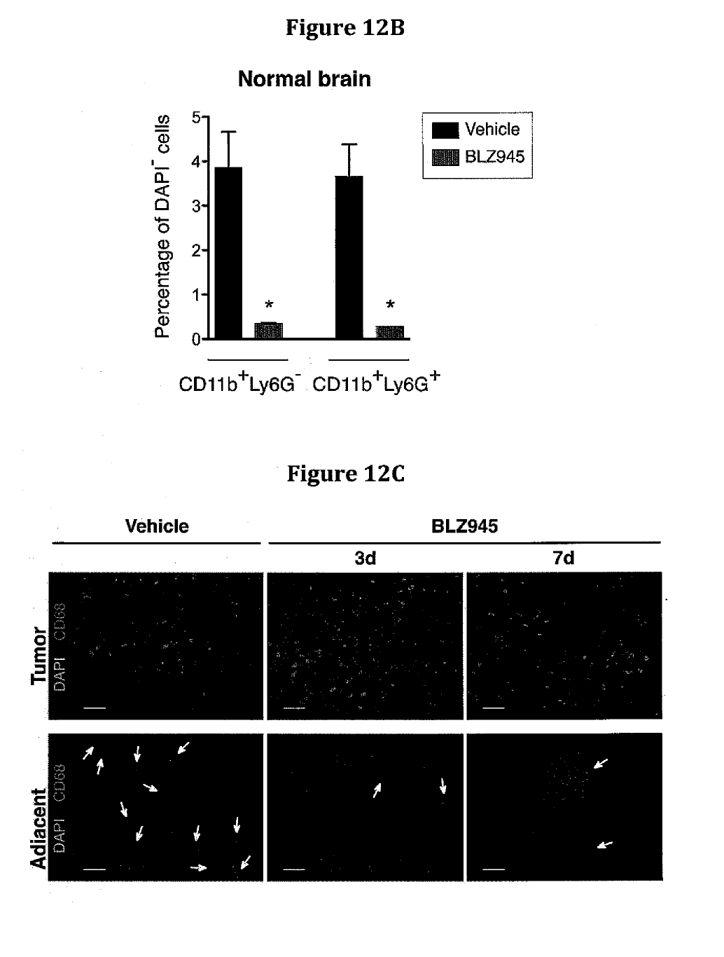

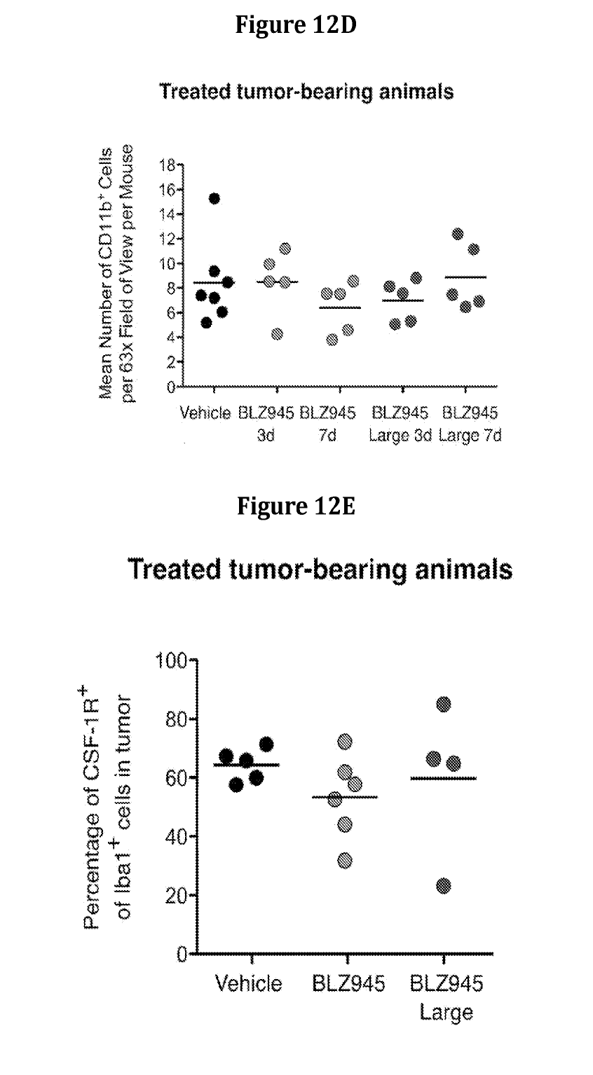

Macrophage survival has been shown to depend on CSF-1R signaling such that inhibition of CSF-1R would be expected to deplete TAM populations. Tumors from each treatment group were stained for macrophage markers and surprisingly, in this context, CSF-1R inhibition did not affect TAM numbers compared to vehicle, despite evident microglia depletion in the adjacent normal brain (FIG. 12). To investigate if CSF-1R inhibitor treatment might select for a CSF-1R independent macrophage population, tumors from d7 of the trial were co-stained for CSF-1R and Iba1; however, there were no significant differences between the treatment groups (FIG. 12E).

### Molecular Mechanism

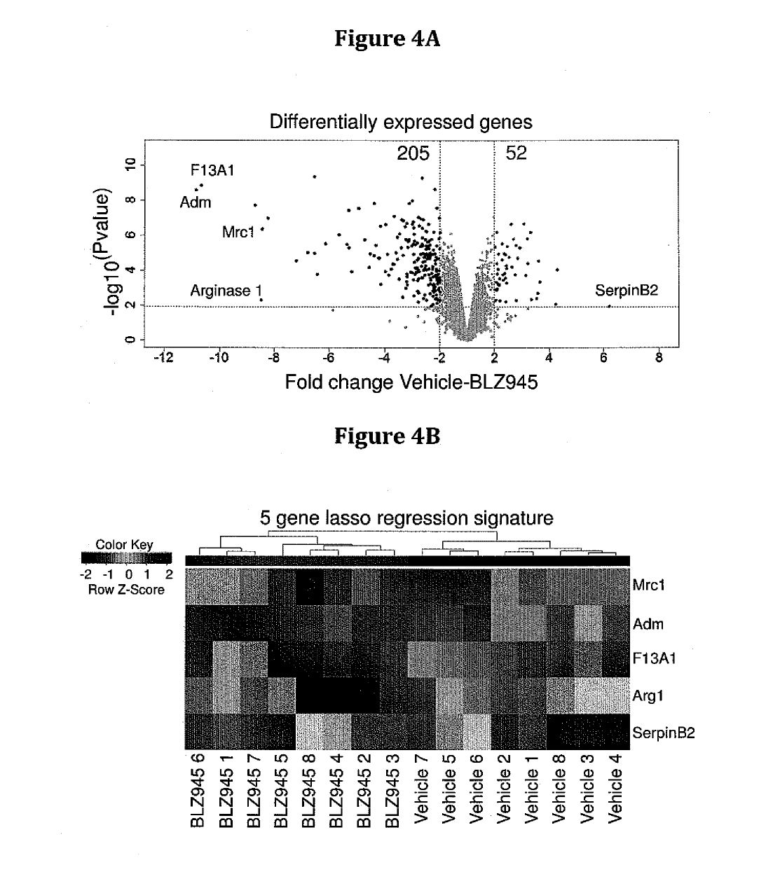

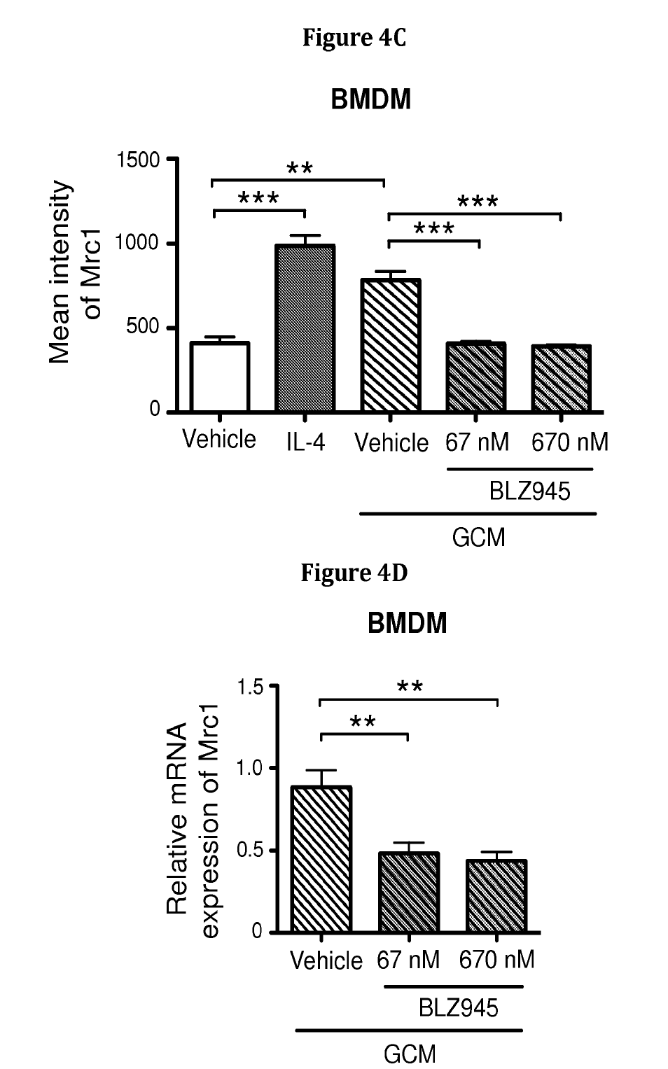

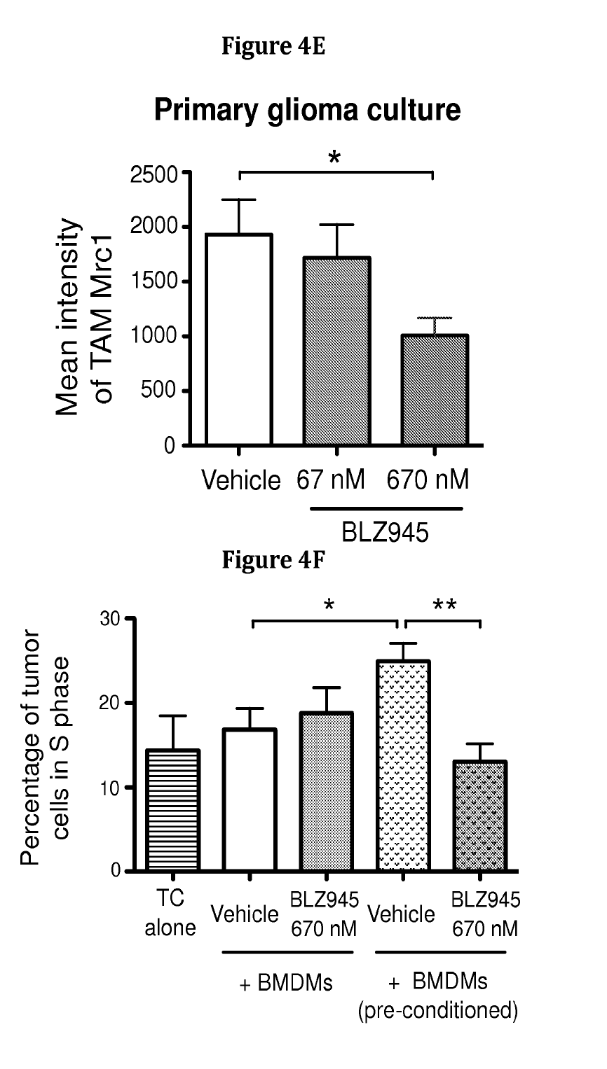

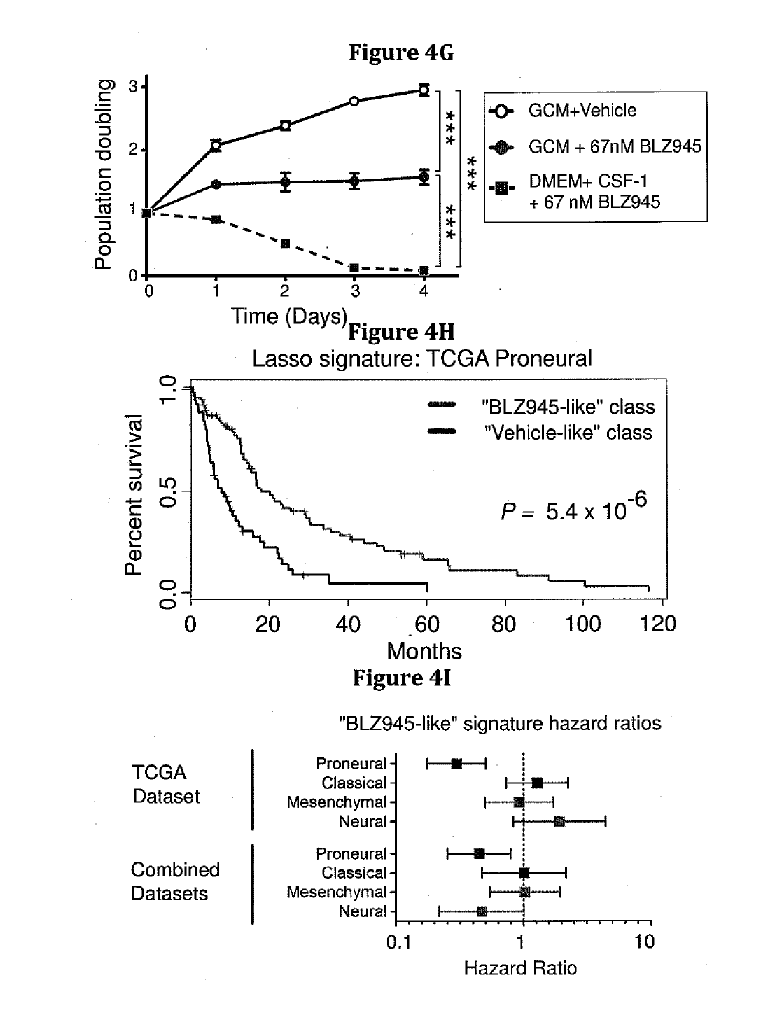

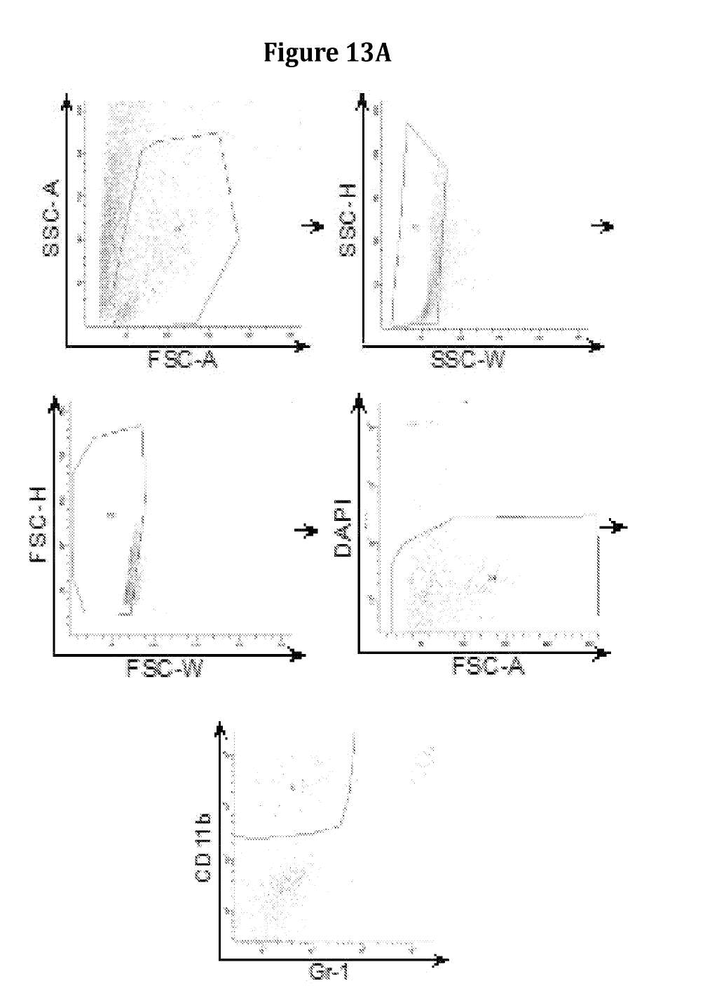

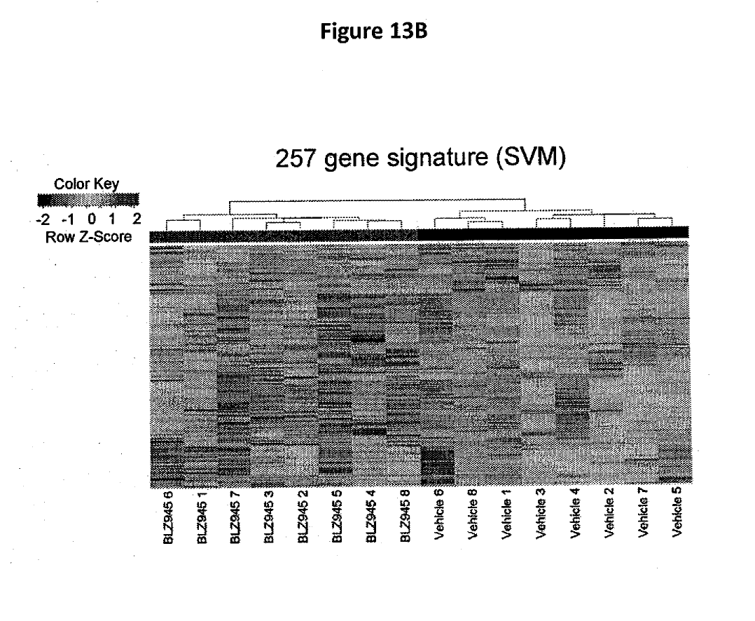

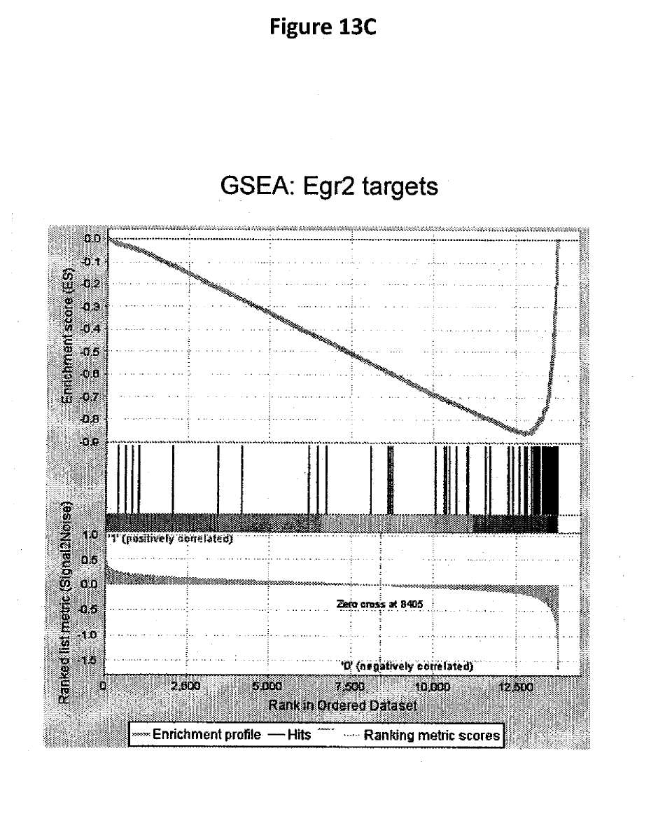

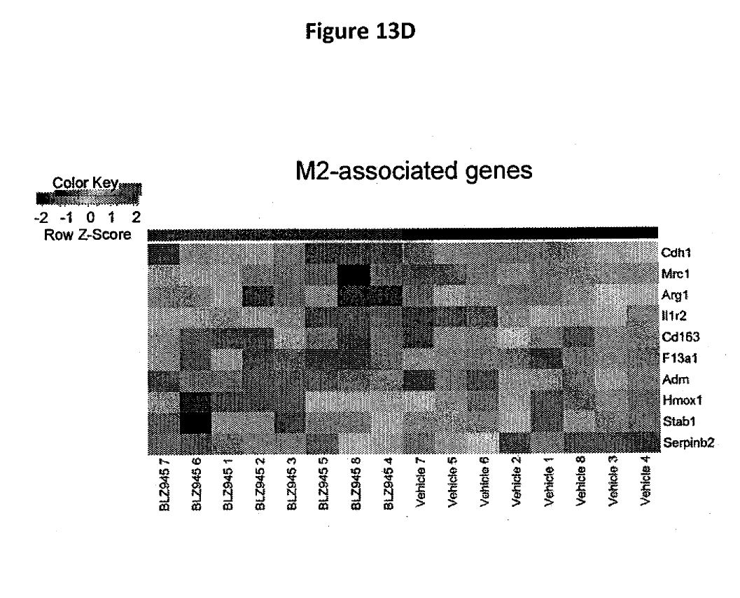

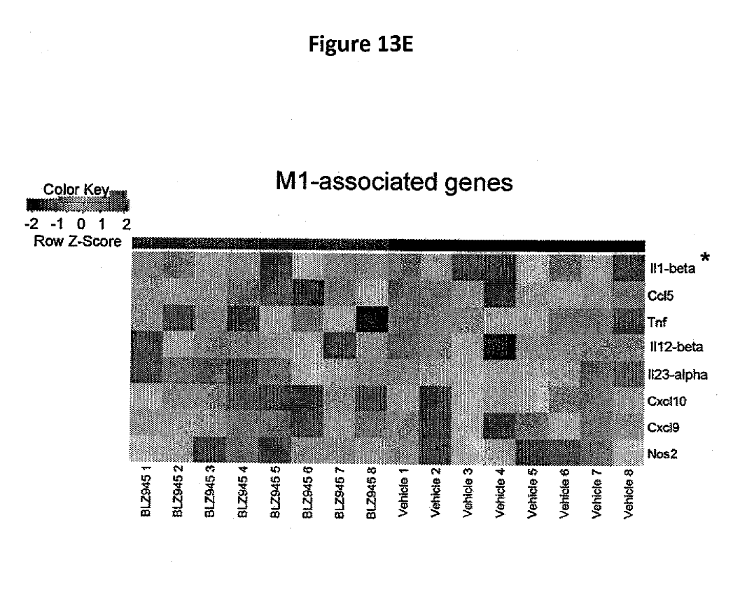

To investigate the molecular mechanisms whereby the CSF-1R inhibitor-treated TAMs can elicit such a striking anti-tumor response in vivo, despite a lack of evident depletion, CD11b⁺Gr-1⁻ TAMs were isolated from vehicle or CSF-1R inhibitor-treated mice and gene expression profiling was performed (FIG. 13). Microarray analysis identified 257 genes as significantly differentially expressed between the groups: 52 genes were upregulated and 205 downregulated (FIGS. 4A and 13). These data were corroborated by the fact that targets of Egr2, a transcription factor downstream of CSF-1R signaling, were downregulated in the CSF-1R inhibitor-treated TAMs (FIG. 13C).

Lasso regression modeling was then used to determine the minimal number of genes that best discriminated the two treatment groups. This identified a 5-gene signature for CSF-1R inhibitor treatment comprised of adrenomedullin (Adm), arginase 1 (Arg1), the clotting factor F13a1, mannose receptor C type 1 (Mrc1/CD206), and the protease inhibitor serpinB2 (FIG. 4B). Interestingly, each of these genes has been associated with alternatively activated/M2 macrophage polarization, and 4 of the 5 genes are downregulated following CSF-1R inhibitor treatment. SerpinB2 (also known as PAI2), the only upregulated gene in the 5-gene signature, generally positively correlates with increased survival, particularly in breast cancer patients.

In many tissue contexts TAMs have been found to be more M2 polarized, which has been linked to their immunosuppressive and pro-tumorigenic functions. Furthermore, macrophages in human gliomas exhibit an M2-like phenotype, determined by increased levels of the scavenger receptors CD163 and CD204, which are associated with higher tumor grade. Given the striking enrichment for M2 genes in the restricted 5-gene signature, the 257-gene list was examined to determine if there were additional M2-associated markers altered following CSF-1R inhibitor treatment. This revealed 10 more genes, the majority of which were downregulated (FIG. 13, Table 2). Classically activated/M1 polarization genes were not correspondingly upregulated (FIG. 13). These data suggest that in response to CSF-1R inhibition, TAMs lose their M2 polarization and may gain anti-tumorigenic functions.

Loss of M2 markers could be associated with immunostimulatory effects of macrophages on the immune system; thus immune cell infiltration of vehicle and CSF-1R inhibitor-treated tumors were compared by flow cytometry. However, no differences were observed for natural killer cells, CD8⁺ or CD4⁺ T cells, nor CD19⁺ B cells, which each comprise <1% of the cells isolated from the tumor (FIG. 14). These data cannot rule out the involvement of the adaptive immune system in TAM-mediated responsiveness to the CSF-1R inhibitor, but are not strongly indicative of such an effect.

To further examine the mechanisms by which the CSF-1R inhibitor elicits a striking anti-tumor response, different cell-based assays were performed. First, BMDMs was exposed to glioma cell-conditioned media (GCM) to model the glioma microenvironment, and expression of Mrc1 from the 5-gene signature was examined (FIG. 4B). Mrc1 was selected as it is a well-established cell surface M2 marker, facilitating the use of flow cytometry to examine levels of macrophages in co-culture assays. Mrc1 increased in response to GCM, and was downregulated following CSF-1R inhibitor addition (FIGS. 4C-D). Similarly, in freshly isolated mouse primary glioma cultures containing TAMs (FIG. 15), Mrc1 was also decreased in response to the CSF-1R inhibitor (FIG. 4E). Given the parallels with the downregulation of M2 markers in vivo, whether tumor cell proliferation was similarly affected in co-culture with macrophages and the CSF-1R inhibitor was examined. Cell cycle analysis showed that the addition of GCM-prestimulated BMDMs to glioma cells increased their proliferation, which was reduced by the addition of the CSF-1R inhibitor (FIG. 4F). Interestingly, it was also found that GCM protected BMDMs from CSF-1R inhibitor-induced death in culture (FIG. 4G), analogous to the maintenance of TAMs in vivo (FIG. 12). Together, these data show that macrophages and tumor cells have reciprocal effects on survival and/or proliferation of the other cell type, and this heterotypic signaling can be perturbed by CSF-1R inhibition.

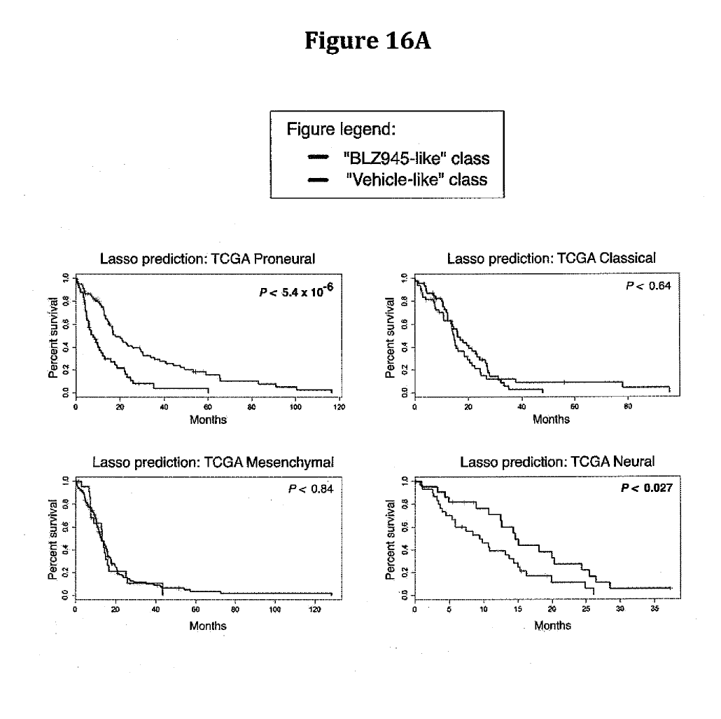

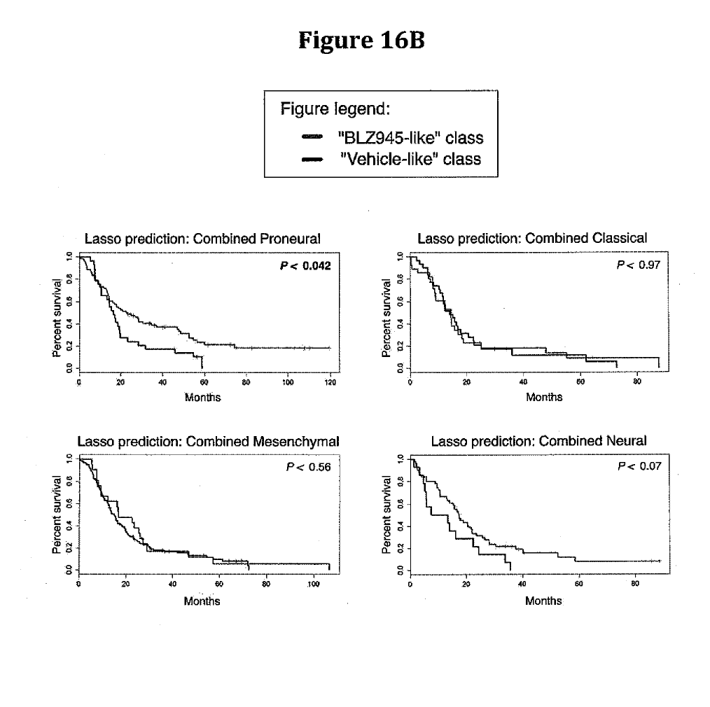

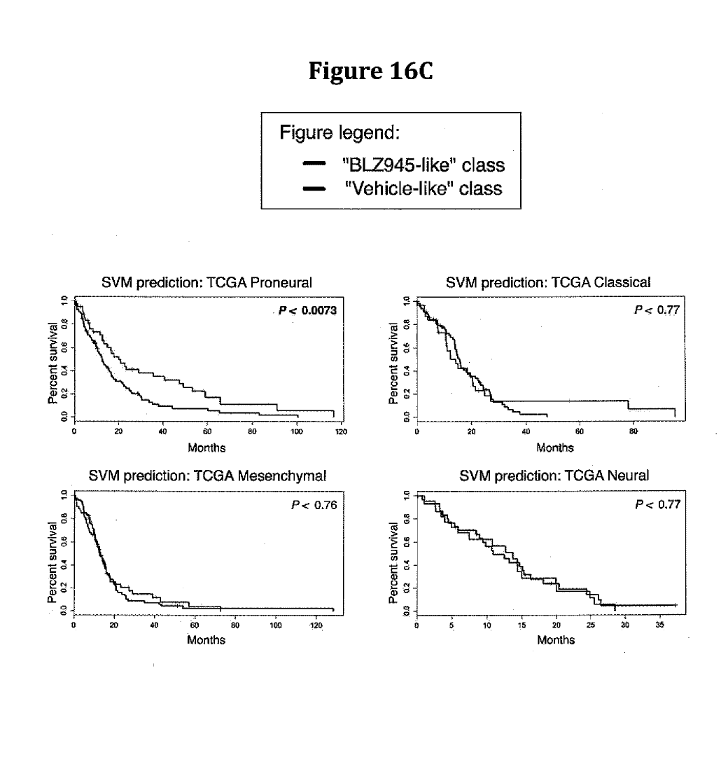

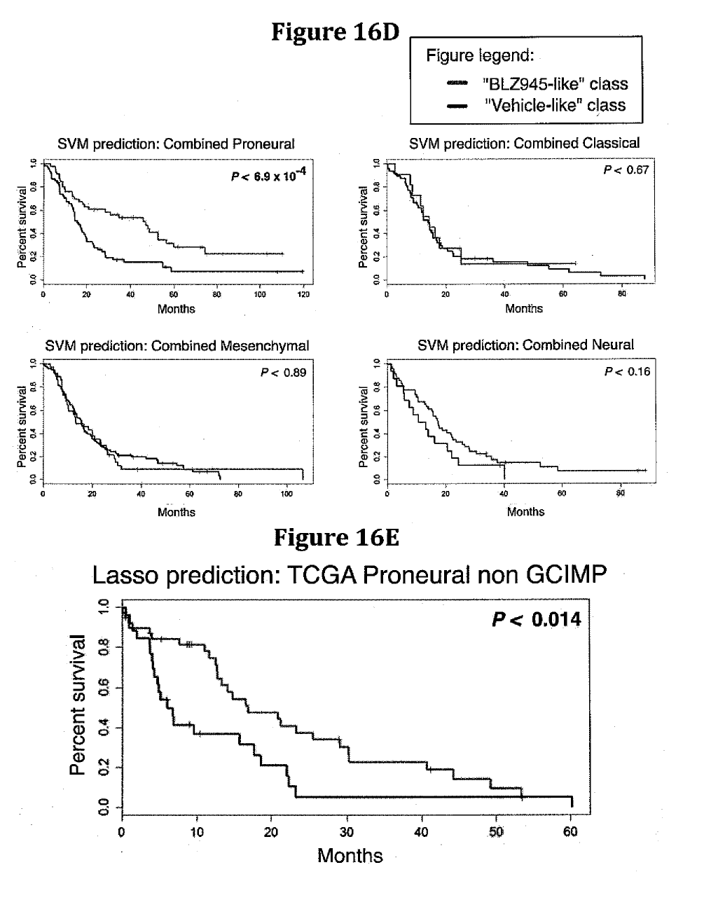

Finally, it was determined whether the gene signatures generated from the CSF-1R inhibitor-treated TAMs in mice might be associated with differential survival in GBM patients. A support vector machine (SVM) and the Lasso signature were used to analyze GBM TCGA and a second combined series of GBM datasets (see method in Example 1), and segregated patients into either ‘Treatment (BLZ945)’ or ‘Vehicle’ classifiers. These analyses revealed an increase in median survival ranging from 10 months in TCGA proneural patients using the Lasso signature (FIG. 4H) to 31.5 months in the combined datasets with the SVM signature (FIG. 16, Table 3). Interestingly, this increase in survival was not evident in other subtypes of GBM, and was not dependent upon enrichment of G-CIMP⁺ proneural patients (FIG. 15, Table 4). Analysis of associated hazard ratios demonstrated the proneural-specific survival advantage in both TCGA and the combined data sets (FIG. 41, Table 5). The proneural specificity is consistent with the TAM signatures originally having been generated from the PDG model of gliomagenesis, which most closely represents proneural GBM. As proneural GBM does not respond to aggressive chemo-and radiotherapy compared to the other subtypes, the finding of prognostic value associated with these signatures may have important translational potential for this group of patients.

TABLE 1
Summary of Histological Analyses Performed In Each Treatment Group

| | | | | BLZ945 | BLZ945 |
|---|---|---|---|---|---|
| | | BLZ945, | BLZ945, | Large, | Large, |
| Parameter | Vehicle | Day 3 | Day 7 | Day 3 | Day 7 |
| Tumor Volume | +498% | — | +0.68% | — | −24.3% |
| (Day −1 vs Day 6) | | | | | |
| Total DAPI⁺ Cells | — | −72% | −80% | −40% | −65% |
| Tumor Cells | — | −27% | −77% | −14% | −73% |
| (% Olig2⁺) | | | | | |
| Proliferation | — | −91% | −67% | −98% | −94% |
| (% BrdU⁺Olig2⁺) | | | | | |
| Apoptosis (% CC3⁺) | — | +17-fold | +6-fold | +9-fold | +2-fold |
| Vasculature | — | — | −17% | — | −67% |
| (CD31 MVD) | | | | | |
| Macrophages | — | +3-fold | +2-fold | +2-fold | +4-fold |
| (% CD68⁺) | | | | | |
| Phagocytic | — | +2.6-fold | +3.0-fold | +2.2-fold | +4.1-fold |
| Index | | | | | |
| Phagocytic | — | +11.5-fold | +5.0-fold | +7.1-fold | +6.0-fold |
| Capacity | | | | | |

All changes in the BLZ945 treatment groups are calculated relative to the vehicle control group. MVD: microvessel density.

TABLE 2
A Lilst of 257 Differentially Expressed Genes From Microarray Analysis of BLZ945-Treated TAMs

| | | Fold Change | Nominal |
|---|---|---|---|
| Symbol | Description | BLZ945-Vehicle | P value |
| 2310016C08Rik | RIKEN cDNA 2310016C08 gene | −2.14 | 1.12E−04 |
| 2810417H13Rik | RIKEN cDNA 2810417H13 gene | −3.96 | 2.33E−07 |
| 4930583H14Rik | RIKEN cDNA 4930583H14 gene | −2.37 | 1.25E−05 |
| Abhd15 | abhydrolase domain containing 15 | −2.48 | 1.36E−05 |
| Acp5 | acid phosphatase 5, tartrate resistant | −2.36 | 2.08E−03 |
| Ada | adenosine deaminase | −3 | 2.28E−07 |
| Adm *, ** | adrenomedullin | −10.85 | 2.60E−09 |
| Akap12 | A kinase (PRKA) anchor protein (gravin) 12 | −2.85 | 1.31E−04 |
| Aldh1a2 | aldehyde dehydrogenase family 1, subfamily A2 | −2.18 | 8.36E−04 |
| Alox15 | arachidonate 15-lipoxygenase | 4.24 | 8.85E−03 |
| Anln | anillin actin binding protein | −2.99 | 1.38E−04 |
| Aoah | acyloxyacyl hydrolase | −2.43 | 3.83E−06 |
| Apbb2 | amyloid beta (A4) precursor protein-binding, family | 2.27 | 2.97E−06 |
| | B, member 2 | | |
| Apob | apolipoprotein B | −2.92 | 3.42E−05 |
| Apoc1 | apolipoprotein C-I | 3.21 | 1.56E−06 |
| Apoc4 | apoplipoprotein C-IV | 3.14 | 1.91E−04 |
| Arg1 *, **,# | arginase, liver | −8.48 | 5.07E−03 |
| Arxes1 | adipocyte-related X-chromosome expressed sequence 1 | −2.23 | 1.68E−03 |
| Arxes2 | adipocyte-related X-chromosome expressed sequence 2 | −2.96 | 3.37E−04 |
| Asb10 | ankyrin repeat and SOCS box-containing 10 | 2.1 | 1.14E−03 |
| Asb11 | ankyrin repeat and SOCS box-containing 11 | 2.19 | 3.00E−04 |
| Aspm | asp (abnormal spindle-like, microcephaly | −2.22 | 1.02E−03 |
| | associated (Drosophila) | | |
| Aurka | aurora kinase A | −2.23 | 1.30E−03 |
| Aurkb | aurora kinase B | −2.71 | 4.19E−06 |
| Bambi | BMP and activin membrane-bound inhibitor, homolog (Xenopus laevis) | 2.64 | 6.53E−05 |
| Birc5 | baculoviral IAP repeat-containing 5 | −6.13 | 3.00E−06 |
| Bub1 | budding uninhibited by benzimidazoles 1 homolog (S. cerevisiae) | −2.72 | 4.19E−06 |
| Calml4 | calmodulin-like 4 | −2.06 | 2.12E−05 |
| Camkk1 | calcium/calmodulin-dependent protein kinase | −2.13 | 2.69E−08 |
| | kinase 1, alpha | | |
| Cbr2 | carbonyl reductase 2 | −4.15 | 2.93E−07 |
| Ccna2 | cyclin A2 | −3.9 | 1.19E−05 |
| Ccnb1 | cyclin B1 | −3.55 | 2.25E−05 |
| Ccnb2 | cyclin B2 | −4.53 | 1.16E−05 |
| Ccnd1 | cyclin D1 | −3.01 | 1.06E−08 |
| Ccnd2 | cyclin D2 | −3.34 | 1.36E−05 |
| Ccne2 | cyclin E2 | −5.28 | 3.67E−08 |
| Ccnf | cyclin F | −2.3 | 1.48E−04 |
| Ccr1 | chemokine (C-C motif) receptor 1 | −4.56 | 6.86E−05 |
| Cd163 ** | CD163 antigen | −2.65 | 3.87E−07 |
| Cd22 | CD22 antigen | 2.35 | 1.09E−05 |
| Cd244 | CD244 natural killer cell receptor 2B4 | −2.71 | 1.11E−07 |
| Cd38 | CD38 antigen | −3.72 | 4.44E−05 |
| Cd5 | CD5 antigen | 3.62 | 2.96E−05 |
| Cd83 | CD83 antigen | 2.28 | 2.53E−05 |
| Cd93 | CD93 antigen | −2.42 | 2.30E−07 |
| Cdc20 | cell division cycle 20 homolog (S. cerevisiae) | −2.75 | 1.16E−04 |
| Cdc45 | cell division cycle 45 homolog (S. cerevisiae) | −2.03 | 9.78E−08 |
| Cdc6 | cell division cylce 6 homolog (S. cerevisae) | −3.67 | 8.12E−08 |
| Cdca5 | cell division cycle associated 5 | −2.24 | 6.77E−06 |
| Cdh1 ** | cadherin 1 | −6.43 | 1.70E−04 |
| Cdh2 | cadherin 2 | −2.23 | 6.25E−04 |
| Cdk1 | cyclin-dependent kinase 1 | −2.18 | 2.75E−05 |
| Cenpe | centromere protein E | −4.18 | 1.96E−05 |
| Cenpk | centromere protein K | −2.45 | 1.46E−05 |
| Cep55 | centrosomal protein 55 | −2.4 | 8.23E−05 |
| Cfp | complement factor properdin | −2.64 | 2.60E−04 |
| Chst2 | carbohydrate sulfotransferase 2 | 2.44 | 5.14E−04 |
| Ckap2 | cytoskeleton associated protein 2 | −2.17 | 1.80E−04 |
| Cks1b | CDC28 protein kinase 1b | −2.54 | 1.71E−06 |
| Clec4n | C-type lectin domain family 4, member n | −6.53 | 4.34E−10 |
| Clu | clusterin | −2.34 | 3.55E−04 |
| Cntn1 | contactin 1 | −4.93 | 2.80E−08 |
| Col11a1 | collagen, type XI, alpha 1 | −3.49 | 3.09E−04 |
| Col14a1 | collagen, type XIV alpha 1 | −2.65 | 1.37E−06 |
| Cpa3 | carboxypeptidase A3, mast cell | 2.17 | 6.30E−04 |
| Cpeb1 | cytoplasmic polyadenylation element binding | 2.86 | 1.97E−05 |
| | protein 1 | | |
| Cpne2 | copine II | −2.2 | 1.13E−05 |
| Crybb1 | crystallin, beta B1 | −2.83 | 2.44E−05 |
| Cspg5 | chondroitin sulfate proteoglycan 5 | −2.61 | 1.09E−05 |
| Cst7 | cystatin F (leukocystatin) | 2.62 | 2.29E−07 |
| Ctnnd2 | catenin (cadherin associated protein), delta 2 | −2.94 | 8.46E−07 |
| Ctsf | cathepsin F | 2.1 | 1.53E−04 |
| Cxcr7 | chemokine (C-X-C motif) receptor 7 | −2.26 | 8.65E−03 |
| Cyp4v3 | cytochrome P450, family 4, subfamily v, | 2.14 | 1.15E−05 |
| | polypeptide 3 | | |
| D17H6S56E-5 | DNA segment, Chr 17, human D6S56E 5 | −2.01 | 1.66E−03 |
| Dck | deoxycytidine kinase | −2.07 | 2.50E−04 |
| Ddhd1 | DDHD domain containing 1 | 2.06 | 4.86E−03 |
| Ddit4 | DNA-damage-inducible transcript 4 | −2.43 | 5.07E−06 |
| Depdc1a | DEP domian containing 1a | −2.81 | 9.05E−05 |
| Dhfr | dihydrofolate reductase | −2.4 | 5.79E−06 |
| Dner | delta/notch-like EGF-related receptor | −2.68 | 2.65E−04 |
| Dusp1 | dual specificity phosphatase 1 | 2.33 | 3.55E−04 |
| E2f8 | E2F transcription factor 8 | −2.71 | 1.20E−05 |
| Ect2 | ect2 oncogene | −3.19 | 1.65E−04 |
| Eepd1 | endonuclease/exonuclease/phosphatase family | 2.7 | 1.62E−06 |
| | domain containing 1 | | |
| Emb | embigin | −2.59 | 9.66E−05 |
| Emp1 | epithelial membrance protein 1 | −3.19 | 6.42E−04 |
| Ephx1 | epoxide hydrolase 1, microsomal | 2.76 | 1.75E−04 |
| Eps8 | epidermal growth factor receptor pathway substrate 8 | −2.51 | 4.00E−06 |
| Ero1l | ERO1-like (S. cerevisiae) | −2.64 | 1.07E−05 |
| Etl4 | enhancer trap locus 4 | 2.41 | 1.24E−05 |
| Ezh2 | enhancer of zeste homolog 2 (Drosophila) | −2.54 | 1.36E−05 |
| F13a1 *, ** | coagulation factor XIII, A1 subunit | −10.66 | 1.39E−09 |
| F3 | coagulation factor III | −2.11 | 4.58E−03 |
| F9 | coagulation factor IX | 2.12 | 5.92E−04 |
| Fabp3 | fatty acid binding protein 3, muscle and heart | 2.93 | 4.99E−06 |
| Fabp7 | fatty acide binding protein 7, brain | −6.77 | 9.66E−06 |
| Fam20c | family with sequence similarity 20, member C | 2.79 | 3.62E−06 |
| Fap | fibroblast activation protein | −2.25 | 1.46E−03 |
| Fbn2 | fibrillin 2 | −2.13 | 3.89E−03 |
| Fbxo32 | F-box protein 32 | 2.54 | 1.79E−05 |
| Fhl1 | four and a half LIM domains 1 | −2.02 | 5.40E−03 |
| Fpr2 | formyl peptide receptor 2 | −2.83 | 6.68E−05 |
| Gadd45a | growth arrest and DNA-damage-inducible 45 alpha | 2.4 | 2.77E−04 |
| Gap43 | growth associated protein 43 | −2.56 | 7.42E−05 |
| Gdf3 | growth differentiation factor 3 | −3.33 | 1.40E−07 |
| Gem | GTP binding protein (gene overexpressed in skeletal muscle) | 2.17 | 7.03E−04 |
| Ggta1 | glycoprotein galactosyltransferase alpha 1, 3 | −2.4 | 2.12E−06 |
| Gja1 | gap junction protein, alpha 1 | −2.78 | 1.97E−03 |
| Gpm6a | glycoprotein m6a | −5.35 | 3.23E−06 |
| Gpnmb | glycoprotein (transmembrane) nmb | 3.22 | 3.98E−05 |
| Gzma | granzyme A | 3.55 | 4.11E−03 |
| Hells | helicase, lymphoid specific | −3.59 | 9.76E−06 |
| Hmgb3 | high mobility group box 3 | −2.42 | 2.32E−06 |
| Hmgn5 | hign-mobility group nucleosome binding domain 5 | −2.58 | 1.79E−05 |
| Hmox1 ** | heme oxygenase (decycling) 1 | −2.9 | 7.05E−05 |
| Hsp90aa1 | heat shock protein 90, alpha (cytosolic), class A member 1 | −2.23 | 1.01E−03 |
| Hspa1a | heat shock protein A | −4.38 | 1.45E−05 |
| Hspa1b | heat shock protein 1B | −8.71 | 1.88E−08 |
| Ifitm1 | interferon induced transmembrane protein 1 | −5.18 | 1.21E−04 |
| Ifitm2 | interferon induced transmembrane protein 2 | −2.82 | 1.54E−03 |
| Ifitm3 | interferon induced transmembrane protein 3 | −2.06 | 4.73E−03 |
| Ifitm6 | interferon induced transmembrane protein 6 | −4.14 | 6.14E−04 |
| Igf1 | insulin-like growth factor 1 | 2.13 | 8.56E−05 |
| Igfbp2 | insulin-like growth factor binding protein 2 | −3.54 | 1.24E−06 |
| Igfbp3 | insulin-like growth factor binding protein 3 | −6.53 | 1.05E−05 |
| Igj | immunoglobulin joining chain | 3.36 | 4.53E−03 |
| Ikbke | inhibitor of kappaB kinase epsilon | 2.38 | 1.50E−04 |
| Il1r2 ** | interleukin 1 receptor, type II | −2.32 | 1.9E−02 |
| Il18bp | interleukin 18 binding protein | 3.66 | 4.53E−04 |
| Il1b *** | interleukin 1 beta | 2.06 | 4.50E−04 |
| Il7r | interleukin 7 receptor | −2.01 | 4.96E−03 |
| Itgam | integrin alpha M | −2.25 | 2.27E−04 |
| Itgax | integrin alpha X | 2.08 | 1.36E−03 |
| Kcnk2 | potassium channel, subfamily K, member 2 | −2.13 | 8.52E−05 |
| Khdrbs3 | KH domain containing, RNA binding, signal | −2.1 | 3.94E−04 |
| | transduction associated 3 | | |
| Kif11 | kinesin family member 11 | −2.57 | 9.00E−05 |
| Kif20a | kinesin family member 20A | −3.76 | 8.28E−05 |
| Kif22 | kinesin family member 22 | −2.14 | 2.42E−04 |
| Klrb1a | killer cell lectin-like receptor subfamily B member | 4.3 | 9.00E−05 |
| | 1A | | |
| Kpna2 | karyopherin (importin) alpha 2 | −2.36 | 1.98E−05 |
| Lgals1 | lectin, galactose binding, soluble 1 | −2.11 | 3.24E−04 |
| Lgals9 | lectin, galactose binding, soluble 9 | −2.42 | 6.75E−04 |
| Lifr | leukemia inhibitory factor receptor | −2.06 | 1.12E−03 |
| Lig1 | ligase I, DNA ATP-dependent | −2.66 | 5.22E−10 |
| Lox | lysyl oxidase | 2.42 | 6.19E−03 |
| Lpl | lipoprotein lipase | 3.08 | 2.21E−07 |
| Lrr1 | leucine rich repeat protein 1 | −2.65 | 3.08E−06 |
| Ltc4s | leukotriene C4 synthase | −2.52 | 5.69E−07 |
| Mad2l1 | MAD2 mitotic arrest deficient-like 1 (yeast) | −2.45 | 1.17E−05 |
| Mcm2 | minichromosome maintenance deficient | −2.79 | 9.32E−08 |
| | 2 mitotin (S. cerevisiae) | | |
| Mcm4 | minichromosome maintenance deficient | −2.93 | 2.05E−06 |
| | 4 homolog (S. cerevisiae) | | |
| Mcm5 | minichromosome maintenance deficient | −4.38 | 1.47E−08 |
| | 5, cell division cycle 46 (S. cerevisiae) | | |
| Mcm6 | minichromosome maintenance deficient | −3.27 | 3.13E−07 |
| | 6 (MIS5 homolog, S. pombe) (S. cerevisiae) | | |
| Mcm7 | minichromosome maintenance deficient | −2.23 | 6.52E−07 |
| | 7 (S. cerevisiae) | | |
| Melk | maternal embryonic leucine zipper kinase | −3.4 | 5.03E−06 |
| Mis18bp1 | MIS18 binding protein 1 | −2.03 | 1.03E−04 |
| Mitf | microphthalmia-associated transcription factor | 2.08 | 6.51E−07 |
| Mki67 | antigen identified by monoclonal antibody Ki 67 | −7.18 | 2.78E−05 |
| Mmp10 | matrix metallopeptidase 10 | −2.33 | 1.11E−02 |
| Moxd1 | monooxygenase, DBH-like 1 | −2.81 | 3.72E−04 |
| Mrc1 *, ** | mannose receptor, C type 1 (CD206) | −8.44 | 4.40E−07 |
| Ms4a4c | membrane-spanning 4-domains, subfamily A, member 4C | −2.11 | 7.53E−03 |
| Ms4a6b | membrane-spanning 4-domains, subfamily A, member 6B | −3.12 | 1.45E−08 |
| Ms4a6c | membrane-spanning 4-domains, subfamily A, member 6C | −2.47 | 2.34E−06 |
| Ms4a7 | membrane-spanning 4-domains, subfamily A, member 7 | −3.2 | 2.00E−07 |
| Mt2 | metallothionein 2 | −2.17 | 1.44E−03 |
| Mtss1 | metastasis suppressor 1 | −2.28 | 1.19E−05 |
| Nap1l5 | nucleosome assembly protein 1-like 5 | −2.91 | 1.63E−04 |
| Ncapd2 | non-SMC condensin I complex, subunit D2 | −2.77 | 1.98E−05 |
| Ncapg | non-SMC condensin 1 complex, subunit G | −3.02 | 3.89E−06 |
| Ncapg2 | non-SMC condensin II complex, subunit G2 | −2.96 | 3.08E−06 |
| Ndc80 | NDC80 homolog, kinetochore complex | −2.28 | 8.12E−05 |
| | component (S. cerevisiae) | | |
| Nkg7 | natural killer cell group 7 sequence | 2.04 | 5.16E−03 |
| Nop58 | NOP58 ribonucleoprotein homolog (yeast) | −2.14 | 2.49E−03 |
| Nov | nephroblastoma overexpressed gene | 3.6 | 1.99E−03 |
| Nrp2 | neuropilin 2 | 2.02 | 4.53E−06 |
| Nt5dc2 | 5′-nucleotidase domain containing 2 | −2.48 | 2.23E−05 |
| Nuf2 | NUF2, NDC80 kinetochore complex | −5.28 | 5.04E−06 |
| | component, homolog (S. cerevisiae) | | |
| Olig1 | oligodendrocyte transcription factor 1 | −3.85 | 1.82E−04 |
| Olig2 | oligodendrocyte transcription factor 2 | −2.35 | 2.75E−04 |
| P2ry12 | purinergic receptor P2Y, G-protein coupled 12 | −2.55 | 1.86E−04 |
| P4ha2 | procollagen-proline, 2-oxoglutarate 4-dioxygenase | −3.54 | 1.25E−06 |
| | (proline 4-hydroxylase), alpha II polypeptide | | |
| Pbk | PDZ binding kinase | −5.63 | 9.20E−07 |
| Pdgfc | platelet-derived growth factor, C polypeptide | −2.25 | 2.98E−03 |
| Pdgfra | platelet derived growth factor receptor, alpha polypeptide | −3.16 | 4.84E−06 |
| Pdpn | podoplanin | −2.01 | 3.51E−04 |
| Pf4 | platelet factor 4 | −2.96 | 1.21E−05 |
| Pilra | paired immunoglobin-like type 2 receptor alpha | −2.35 | 1.51E−05 |
| Plac8 | placenta-specific 8 | −2.79 | 6.64E−03 |
| Plk1 | polo-like kinase 1 (Drosophila) | −2.68 | 5.72E−05 |
| Pmepa1 | prostate transmembrane protein, androgen induced 1 | −2.35 | 5.85E−04 |
| Pnlip | pancreatic lipase | −2.39 | 1.76E−04 |
| Pola1 | polymerase (DNA directed), alpha 1 | −2.37 | 3.52E−06 |
| Pold2 | polymerase (DNA directed), delta 2, regulatory | −2.02 | 5.72E−06 |
| | subunit | | |
| Pole | polymerase (DNA directed), epsilon | −2.27 | 5.96E−05 |
| Prc1 | protein regulator of cytokinesis 1 | −3.06 | 1.26E−04 |
| Prickle1 | prickle homolog 1 (Drosophila) | 2.32 | 1.75E−04 |
| Prim1 | DNA primase, p49 subunit | −2.76 | 2.87E−07 |
| Psmb7 | proteasome (prosome, macropain) subunit, beta | −2.17 | 4.27E−03 |
| | type 7 | | |
| Ptger4 | prostaglandin E receptor 4 (subtype EP4) | 2.43 | 5.12E−05 |
| Ptn | pleiotrophin | −3.21 | 4.42E−04 |
| Ptprz1 | protein tyrosin phosphatase, receptor type Z, | −3.66 | 4.43E−05 |
| | polypeptide 1 | | |
| Pttg1 | pituitary tumor-transforming gene 1 | −2.83 | 2.73E−06 |
| Rab34 | RAB34, member of RAS oncogene family | 2.08 | 3.95E−05 |
| Racgap1 | Rac GTPase-activating protein 1 | −2.56 | 2.92E−05 |
| Rad51 | RAD51 homolog (S. cerevisiae) | −2.9 | 3.33E−06 |
| Rad51ap1 | RAD51 associated protein 1 | −2.61 | 1.26E−07 |
| Ranbp1 | RAN binding protein 1 | −2.01 | 6.62E−07 |
| Rbm3 | RNA binding motif protein 3 | −2.2 | 2.23E−09 |
| Rbp1 | retinol binding protein 1, celluluar | −4.22 | 1.88E−05 |
| Rfc4 | replication factor C (activator 1) 4 | −2.17 | 4.24E−05 |
| Rrm1 | ribonucleotide reductase M1 | −2.14 | 1.79E−05 |
| Rrm2 | ribonucleotide reductase M2 | −8.23 | 1.04E−07 |
| Serpinb2 *, ** | serine (cysteine) peptidase inhibitor, clade B, | 6.2 | 1.12E−02 |
| | member 2 (PAI2) | | |
| Serpinb6b | serine (or cysteine) peptidase inhibitor, clade B, | 2.03 | 1.22E−03 |
| | member 6b | | |
| Sh3bgr | SH3-binding domain glutamic acid-rich protein | 3.33 | 6.84E−07 |
| Sh3bgrl | SH3-binding domain glutamic acid-rich protein like | −2.02 | 4.68E−04 |
| Shcbp1 | Shc SH2-domain binding protein 1 | −4.72 | 1.74E−06 |
| Slamf8 | SLAM family member 8 | 2.81 | 5.20E−03 |
| Slc2a5 | solute carrier family 2 (facilitated glucose | −3.46 | 8.15E−07 |
| | transporter), member 5 | | |
| Slc39a4 | solute carrier family 39 (zinc transporter), member 4 | 2.44 | 7.83E−05 |
| Slc6a1 | solute carrier family 6 (neurotransmitter transporter, | −2.09 | 5.66E−04 |
| | GABA), member 1 | | |
| Slfn4 | schlafen 4 | −3.35 | 3.42E−03 |
| Smc2 | structural maintenance of chromosomes 2 | −2.87 | 7.80E−05 |
| Smc4 | structural maintenance of chromosomes 4 | −3.2 | 2.26E−04 |
| Smyd2 | SET and MYND domain containing 2 | −2.25 | 6.90E−04 |
| Snrpa1 | small nuclear ribonucleoprotein polypeptide A′ | −2.04 | 1.48E−06 |
| Sox2 | SRY-box containing gene 2 | −2.5 | 5.13E−03 |
| Sparcl1 | SPARC-like 1 | −3.23 | 1.06E−03 |
| Spon1 | spondin 1, (f-spondin) extracellular matrix protein | −2.5 | 9.32E−05 |
| St14 | suppression of tumorigenicity 14 (colon carcinoma) | 2.47 | 4.21E−06 |
| Stab1 ** | stabilin 1 | −2.64 | 3.92E−06 |
| Stil | Scl/Tal1 interrupting locus | −3.15 | 2.15E−06 |
| Stmn1 | stathmin 1 | −2.52 | 5.52E−05 |
| Tcf19 | transcription factor 19 | −2.32 | 1.67E−06 |
| Tfpi2 | tissue factor pathway inhibitor 2 | −2.6 | 3.99E−03 |
| Tgfbi | transforming growth factor, beta induced | −3.23 | 1.68E−06 |
| Tgm2 | transglutaminase 2, C polypeptide | −2.94 | 2.86E−03 |
| Timp1 | tissue inhibitor of metalloproteinase 1 | −2.07 | 1.64E−04 |
| Tiparp | TCDD-inducible poly(ADP-ribose) polymerase | −2.06 | 1.25E−03 |
| Tipin | timeless interacting protein | −2.5 | 6.24E−06 |
| Tk1 | thymidine kinase 1 | −3.39 | 1.24E−07 |
| Tmem119 | transmembrane protein 119 | −3.12 | 1.71E−06 |
| Tmem163 | transmembrane protein 163 | 2.2 | 5.42E−03 |
| Tnc | tenascin C | −2.56 | 2.48E−03 |
| Top2a | topoisomerase (DNA) II alpha | −2.11 | 2.13E−05 |
| Topbp1 | topoisomerase (DNA) II binding protein 1 | −2.37 | 9.96E−06 |
| Tpx2 | TPX2, microtubule-associated protein homolog (Xenopus laevis) | −2.52 | 1.16E−05 |
| Trim59 | tripartite motif-containing 59 | −2.78 | 3.02E−04 |
| Trps1 | trichorhinophalangeal syndrome I (human) | −2.97 | 1.68E−07 |
| Ttk | Ttk protein kinase | −2.63 | 3.35E−05 |
| Tubb2c | tubulin, beta 2C | −2.13 | 3.84E−06 |
| Ube2c | ubiquitin-conjugating enzyme E2C | −3.98 | 1.12E−04 |
| Uhrf1 | ubiquitin-like, containing PHD and RING finger domains, 1 | −2.96 | 3.73E−07 |
| Ung | uracil DNA glycosylase | −2.5 | 5.81E−07 |
| Wdhd1 | WD repeat and HMG-box DNA binding protein 1 | −2.01 | 2.52E−04 |
| Zwilch | Zwilch, kinetochore associated, homolog | −4.32 | 3.61E−08 |
| | (Drosophila) | | |

* Component of Lasso regression signature of response to BLZ945.
#Relevant M2 macrophage-associated genes.
* Component of lasso regression signature of response to BLZ945.
** M2 macrophage-associated genes.
*** M1 macrophage-associated gene.
#Arginase 1 is associated with M2 macrophage polarization in mice, but not humans.

Differentially expressed genes were identified as described above (257 genes in total). Downregulated genes in the BLZ945 treated group are given a (−) fold change, while upregulated genes are considered positive. Nominal P values were obtained using Student's two tailed t-test.

TABLE 3
Survival Data For The Support Vector Machine (SVM) And
Lasso Models In The Different GBM Patient Populations

| | “BLZ945- | “Vehicle- | Change in | |
|---|---|---|---|---|
| Group | like” | like” | Median Survival | P value |
| SVM Combined Neural | 49 | 16 | 5.42 | 0.159 |
| SVM Combined Proneural | 46 | 62 | 31.54 | 6.86 × 10⁻⁴ |
| SVM Combined Mesenchymal | 37 | 102 | −2.25 | 0.892 |
| SVM Combined Classical | 11 | 48 | 0.40 | 0.667 |
| SVM TCGA Proneural | 45 | 88 | 7.64 | 0.00727 |
| SVM TCGA Proneural G-CIMP | 13 | 8 | −40.60 | 0.201 |
| SVM TCGA Proneural non G-CIMP | 22 | 44 | −0.76 | 0.264 |
| SVM TCGA G-CIMP | 14 | 8 | −35.60 | 0.203 |
| SVM TCGA non G-CIMP | 83 | 157 | −1.06 | 0.727 |
| SVM TCGA Neural | 23 | 30 | 2.84 | 0.773 |
| SVM TCGA Mesenchymal | 53 | 99 | 0.30 | 0.762 |
| SVM TCGA Classical | 31 | 66 | −3.14 | 0.771 |
| Lasso Combined Neural | 51 | 14 | 7.01 | 0.065 |
| Lasso Combined Proneural | 79 | 29 | 6.51 | 0.0415 |
| Lasso Combined Mesenchymal | 21 | 118 | 1.88 | 0.555 |
| Lasso Combined Clssical | 28 | 31 | 0.33 | 0.968 |
| Lasso TCGA Proneural | 84 | 49 | 9.98 | 5.41 × 10⁻⁶ |
| Lasso TCGA Proneural G-CIMP | 20 | 1 | NA | NA |
| Lasso TCGA Proneural non G-CIMP | 40 | 26 | 10.84 | 0.014 |
| Lasso TCGA G-CIMP | 20 | 2 | −16.13 | 0.721 |
| Lasso TCGA non G-CIMP | 100 | 140 | 0.10 | 0.414 |
| Lasso TCGA Neural | 31 | 22 | −5.19 | 0.0272 |
| Lasso TCGA Mesenchymal | 23 | 129 | 0.40 | 0.835 |
| Lasso TCGA Classical | 49 | 48 | −1.42 | 0.634 |

An increase in median survival in the “BLZ945-like” class compared to the “Vehicle-like” class is depicted as a positive value, while a decrease in survival is shown as a negative value. Although TCGA neural patients classified to “BLZ945-like” with the lasso model demonstrated a decrease in survival, this was not seen in the Combined dataset, or with the SVM model in either dataset. Only proneural patients demonstrated a consistent survival advantage in the “BLZ945-like” class in both the TCGA and Combined datasets using either the lasso or SVM model. P values for median survival were obtained using a Chi-squared test, and all significant P values are indicated in bold.

TABLE 4
Hazard Ratios And Associated 95% Confidence Intervals For The Lasso
Regression Model In Different G-CIMP And Non-G-CIMP Patient Groups

| | Patient | | | | |
|---|---|---|---|---|---|
| Strata | Population | Model | Hazard Ratio | 95% CI | P value |
| “BLZ945- | Non-G-CIMP | Univariate | 0.4921 | (0.2766-0.8756) | 0.0063 |
| like” | Proneural* | | | | |
| lasso | | | | | |
| “BLZ945- | All Proneural | Univariate | 0.3937 | (0.2601-0.5961) | 9.729 × 10⁻⁶ |
| like” | | | | | |
| lasso | | | | | |
| G-CIMP | All Proneural | Univariate | 0.3289 | (0.1481-0.7304) | 0.01367 |
| G-CIMP | All Proneural | Multivariate** | 0.4601 | (0.1972-1.0733) | 0.07244 |
| “BLZ945- | All Proneural | Multivariate** | 0.4295 | (0.2304-0.8007) | 0.00783 |
| like” | | | | | |
| lasso | | | | | |

*Set of proneural patients with methylation data that are definitively not G-CIMP positive (67/133 total Proneural TCGA patients.)
**Multivariate cox proportional hazard model using both G-CIMP and ‘BLZ945’ classification as strata.

G-CIMP corresponds to Glioma CpG Island Methylator Phenotype. P values were obtained using Wald's test.

TABLE 5
Hazard Ratios And Associated 95% Confidence Intervals For
The Lasso Regression Model In Different Patient Datasets

| Group | Hazard Ratio | 95% CI | P value |
|---|---|---|---|
| TCGA- Proneural | 0.29 | (0.17-0.50) | 6.32 × 10⁻⁶ |
| TCGA- Classical | 1.28 | (0.73-2.26) | 0.389 |
| TCGA- Mesenchymal | 0.93 | (0.49-1.72) | 0.807 |
| TCGA- Neural | 1.93 | (0.83-4.46) | 0.125 |
| Combined- Proneural | 0.44 | (0.25-0.79) | 0.00597 |
| Combined- Classical | 1.01 | (0.47-2.17) | 0.979 |
| Combined- Mesenchymal | 1.02 | (0.54-1.94) | 0.943 |
| Combined- Neural | 0.46 | (0.22-1.01) | 0.0532 |

Hazard ratios and associated 95% confidence intervals (CI). Of note, although TCGA neural patients classified to the “BLZ945-like” class using the lasso model showed significantly decreased median survival with the Chi-squared test (Table 3), the non-significant hazard ratio demonstrates that at any given time point, this classification does not provide a clear association with survival for neural patients. Only hazard ratios from the proneural subtypes are significant. P values were obtained using Wald's test, and all significant P values are indicated in bold.

TABLE 6
List of Antibodies Used For Immunostaining

| Antibody | Clone | Vendor | Dilution |
|---|---|---|---|
| Goat anti-mouse CD31 | | R&D Systems | 1:100 |
| Mouse anti-human smooth | 1A4 | DakoCytomation | 1:100 |
| muscle actin (SMA) | | | |
| Rabbit anti-cleaved caspase 3 | | Cell Signaling | 1:500 |
| (Asp175) (CC3) | | Technology | |
| Rabbit anti-human CSF-1R | C-20 | Santa Cruz | 1:200 |
| Rabbit anti-Iba1 | | Wako | 1:1000 |
| Rabbit anti-green fluorescent | | Molecular Probes | 1:200 |
| protein (GFP) | | | |
| Rabbit anti-Olig2 | | Millipore/ | 1:200 |
| | | Chemicon | |
| Mouse anti-rat Nestin | | BD Pharmingen | 1:500 |
| Rat anti-mouse CD11b | M1/70 | BD Pharmingen | 1:200 |
| Rat anti-BrdU | BU1/ | Serotec | 1:200 |
| | 75(ICR1) | | |
| Rat anti-mouse CD68 | FA-11 | Serotec | 1:1000 |
| Chicken anti-GFAP | | Abcam | 1:1000 |

TABLE 7
List of Antibodies Used For Flow Cytometry Analysis

| Anitbody | Clone | Vendor | Fluorophore(s) | Dilution |
|---|---|---|---|---|
| CD45 | 30-F11 | BD Pharmingen | FITC, APC, | 1:100-1:200 |
| | | | PE-Cy7 | |
| CD3e | 145-2C11 | BD Pharrningen | PE-Cy7 | 1:250 |
| Gr-1 | RB6-8C5 | BD Pharmingen | FITC | 1:200 |
| CD4 | GK1.5 | BD Pharmingen | PE | 1:1000 |
| CD11b | M1/70 | BD Pharmingen | A488, APC, | 1:200 |
| | | | PE | |
| Ly6G | 1A8 | BD Pharmingen | PE-Cy7 | 1:2000 |
| F4/80 | CI: A3-1 | Serotec | PE | 1:50 |
| CD8a | 53-6.7 | Biolegend | A488 | 1:1000 |
| CD19 | 6D5 | Biolegend | PE | 1:2000 |
| NK1.1 | PK136 | Biolegend | APC | 1:1000 |
| CD206 | MR5D3 | Biolegend | A488 | 1:50 |

### REFERENCES

1. Dai et al., Genes Development 15:1913 (2001).
2. Tchougounova et al., Oncogene 26:6289 (2007).
3. Okabe et al., FEBS letters 407:313 (1997).
4. Shih et al., Cancer Res 64:4783 (2004).
5. Hambardzumyan et al., Transl Oncol 2:89 (2009).
6. Fomchenko et al., PloS ONE 6:e20605 (2011).
7. Steiner et al., Journal of Immunological Methods 237:39 (2000).
8. Gocheva et al., Biological Chemistry 391:937 (2010).
9. Hallett et al., Current Protocols In Neuroscience (Editor Jacqueline N. Crawley et al.), Chapter 1, Unit 1 16 (2008).
10. Jensen and Parmar, Molecular Neurobiology 34:153 (2006).
11. Florey et al., Nature Cell Biology 13:1335 (2011).
12. Irizarry et al., Nucleic Acids Research 31:e15 (2003).
13. Gautier et al., Bioinformatics 20:307 (2004).
14. Smyth, Statistical Applications In Genetics And Molecular Biology 3, Article 3 (2004).
15. Subramanian et al., Proc Natl Acad Sci USA 102:15545 (2005).
16. Friedman et al., Journal of Statistical Software 33:1 (2010).
17. McLendon et al., Nature 455:1061 (2008).
18. Freije et al., Cancer Res 64:6503 (2004).
19. Phillips et al., Cancer Cell 9:157 (2006).
20. Murat et al., Journal of Clinical Oncology 26:3015 (2008).
21. Verhaak et al., Cancer Cell 17:98 (2010).
22. Karatzoglou et al., Methods in R 11 (2004).
23. Noushmehr et al., Cancer Cell 17:510 (2010).
24. Therneau, in R package version 2 36-12 (2012).
25. Warnes et al., R package version 2.10.1 (2011).
26. Burns and Wilks, Expert Opin. Ther. Patents 21:147 (2011).
27. Wen and Kesari, N Engl J Med 359:492 (2008).
28. Bonavia et al., Cancer Res 71:4055 (2011).
29. Balkwill and Mantovani, Semin Cancer Biol (2011).
30. Joyce and Pollard, Nat Rev Cancer 9:239 (2009).
31. Bingle et al., J Pathol 196:254 (2002).
32. Hussain et al., Neuro Oncol 8:261 (2006).
33. Komohara et al., J Pathol 216:15 (2008).
34. Ruffell et al., Trends Immunol (2012).
35. Manthey et al., Mol Cancer Ther 8:3151 (2009).
36. Patel and Player, Curr Top Med Chem 9:599 (2009).
37. Wyckoff et al., Cancer Res 64:7022 (2004).
38. Coniglio et al., Mol Med (2012).
39. Fomchenko et al., PLoS One 6:e20605 (2011).
40. Hambardzumyan et al., Transl Oncol 2:89 (2009).
41. Huse and Holland, Brain Pathol 19:132 (2009).
42. Kong et al., Clin Cancer Res 16:5722 (2010).
43. Wang et al., Int J Toxicol 30:300 (2011).
44. Kilic et al., Cancer Res 60:5143 (2000).
45. Takeuchi et al., Br J Cancer 90:1069 (2004).
46. Shih et al., Cancer Res 64:4783 (2004).
47. Serrano et al., Cell 85:27 (1996).
48. Hanahan and Weinberg, Cell 144:646 (2011).
49. Squatrito et al., Cancer Cell 18:619 (2010).
50. Furnari et al., Genes Dev 21:2683 (2007).
51. Chitu and Stanley, Current Opinion in Immunology 18:39 (2006).
52. Bradley et al., J Biol Chem 283:8055 (2008).
53. Friedman et al., Journal of statistical software 33:1 (2010).
54. Biswas and Mantovani, Nat Immunol 11:889 (2010).
55. Torocsik et al., Thrombosis and Haemostasis 104:709 (2010).
56. Probst-Cousin et al., Clinical Neuropathology 17:79 (1998).
57. Chen et al., Clin Cancer Res 17:7230 (2011).
58. Schroder et al., Critical Reviews In Immunology 31:15 (2011).
59. Solinas et al., J Immunol 185:642 (2010).
60. Croucher et al., Nat Rev Cancer 8:535 (2008).
61. Sica et al., Eur J Cancer 42:717 (2006).
62. McLendon et al., Nature 455:1061 (2008).
63. Freije et al., Cancer Res 64:6503 (2004).
64. Phillips et al., Cancer Cell 9:157 (2006).
65. Murat et al., Journal Of Clinical Oncology 26:3015 (2008).
66. Verhaak et al., Cancer Cell 17:98 (2010).
67. Noushmehr et al., Cancer Cell 17:510 (2010).
68. Bender et al., Cancer Res 70:3557 (2010).
69. Denardo et al., Cancer Discovery 1:54 (2011).
70. Shree et al., Genes Dev 25:2465 (2011).
71. Kioi et al., J Clin Invest 120:694 (2010).
72. Wang et al., Investigation of Correlation Among Safety Biiomarkers in Serum, Histopathological Examination, and Toxicogenomics, Int J Toxicology 30:300 (May 2011).
73. Shen et al., Blood 73(7): 1786-93 (1989).
74. Ashmun et al., Blood 73(3): 827-37 (1989).
75. Kitaura et al., J. Dent. Res. 87(4): 396-400 (2008).
76. Aharinejad et al., Cancer Res. 64(15): 5378-84 (2004).
77. Aharinejad et al., Methods Mol. Biol. 487: 243-66 (2009).
78. Abraham et al., Int. J. Cancer 126(6): 1339-52 (2010).
79. PCT Patent Application Publication WO 2009/112245.
80. PCT Patent Application Publication WO 2011/107553.

What is claimed is:

1. A method of determining whether a brain cancer patient would be responsive to a therapeutic reagent or regimen comprising inhibition of colony stimulating factor-1 (CSF-1) signaling, the method comprises the steps of
a) treating the patient with said therapeutic reagent or regimen;
b) isolating tumor-associated myeloid cells from said patient; and
c) determining expression of one or more genes in said myeloid cells, said genes are selected from the group consisting of adrenomedullin (Adm), arginase 1 (Arg1), clotting factor F13a1, mannose receptor C type 1 (Mrc1/CD206), and protease inhibitor serpinB2, wherein differential gene expression in the myeloid cells treated with said therapeutic reagent or regimen as compared to the myeloid cells which are treated under control condition would indicate that said patient would be responsive to treatment with said therapeutic reagent or regimen.

2. The method of claim 1, wherein said genes further comprise one or more genes selected from the group consisting of CD163, Cadherin 1 (Cdh1), Heme oxygenase 1 (Hmox1), Interleukin 1 receptor type II (IL1 r2), and Stabilin 1 (Stab1).

3. The method of claim 1, wherein gene expressions for Adm, Arg1, clotting factor F13a1, and Mrc1/CD206 are downregulated, and gene expression for serpinB2 is upregulated in the myeloid cells treated with said therapeutic reagent or regimen.

4. The method of claim 1, wherein said therapeutic reagent or regimen comprises an inhibitor of CSF-1R, or a CSF-1R inhibitor and another treatment of cancer.

5. The method of claim 1, wherein the myeloid cells are bone marrow-derived macrophages, tumor-associated macrophages, peripheral macrophage precursors, or monocytes.

6. The method of claim 1, wherein the brain cancer is primary brain cancer or metastatic brain cancer.

7. The method of claim 1, wherein the brain cancer is glioma, glioblastoma multiforme, or glioma with the molecular subtype of proneural.

8. The method of claim 6, wherein the primary brain cancer is astrocytoma, oligodendroglioma, neuroblastoma, medulloblastoma, or ependydoma.

9. A method of screening for a therapeutic reagent or regimen for treating brain cancer, the therapeutic reagent or regimen comprises inhibition of colony stimulating factor-1 (CSF-1) signaling, the method comprises the steps of
a) treating a subject with the therapeutic reagent or regimen; and
b) determining expression of one or more genes in myeloid cells obtained from said subject, said genes are selected from the group consisting of adrenomedullin (Adm), arginase 1 (Arg1), clotting factor F13a1, mannose receptor C type 1 (Mrc1/CD206), and protease inhibitor serpinB2, wherein differential gene expression in said myeloid cells from subject treated with the therapeutic reagent or regimen as compared to myeloid cells from subject that is treated with a control reagent or regimen would indicate that said therapeutic reagent or regimen is useful for treating brain cancer.

10. The method of claim 9, wherein gene expressions for Adm, Arg1, clotting factor F13a1, and Mrc1/CD206 are downregulated, and gene expression for serpinB2 is upregulated in the myeloid cells treated with said therapeutic reagent or regimen.

11. The method of claim 9, wherein said therapeutic reagent or regimen comprises an inhibitor of CSF-1R, or a CSF-1R inhibitor and another treatment of cancer.

12. The method of claim 9, wherein said genes further comprise one or more genes selected from the group consisting of CD163, Cadherin 1 (Cdh1), Heme oxygenase 1 (Hmox1), Interleukin 1 receptor type II (IL1 r2), and Stabilin 1 (Stab1).

13. The method of claim 9, wherein the myeloid cells are bone marrow-derived macrophages, tumor-associated macrophages, peripheral macrophage precursors, or monocytes.

14. The method of claim 9, wherein the brain cancer is primary brain cancer or metastatic brain cancer.

15. The method of claim 9, wherein the brain cancer is glioma, glioblastoma multiforme, or glioma with the molecular subtype of proneural.

16. The method of claim 14, wherein the primary brain cancer is astrocytoma, oligodendroglioma, neuroblastoma, medulloblastoma, or ependydoma.
